(19) United States
(12) Patent Application Publication
(10) Pub. No.: US 2023/0031446 A1
(43) Pub. Date: Feb. 2, 2023

(54) SPLIT-ENZYME SYSTEM TO DETECT SPECIFIC DNA IN LIVING CELLS

(71) Applicant: The Regents of the University of California, Oakland, CA (US)

(72) Inventors: David J. Segal, Oakland, CA (US); Nicholas Heath, Oakland, CA (US); Henriette O'Geen, Oakland, CA (US); Jacob Corn, Oakland, CA (US)

(21) Appl. No.: 17/778,254

(22) PCT Filed: Nov. 23, 2020
(86) PCT No.: PCT/US2020/061861

§ 371 Date: May 19, 2022

Related U.S. Application Data
(60) Provisional application No. 62/939,334, filed on Nov. 22, 2019.

(51) Int. Cl. C12Q 1/6897; C12Q 1/66; C12N 9/22; C12N 15/11; C12N 9/02

(52) U.S. Cl. CPC C12Q 1/6897; C12Q 1/66; C12N 9/22; C12N 15/11; C12N 9/0069; C12N 2310/20; C12N 2800/80; C07K 2319/60

(57) ABSTRACT

The present invention provides methods and compositions for detecting genomic sequences of interest in living cells. In particular, the present disclosure provides a split-enzyme system that works with guide RNAs and RNA-guided nucleases to produce detectable luminescent signals exclusively in the presence of targeted genomic sequences.

### CROSS REFERENCE TO RELATED APPLICATIONS

The present application claims priority to U.S. Provisional Pat. Appl. No. 62/939,334, filed on Nov. 22, 2019, which application is incorporated herein by reference in its entirety.

### REFERENCE TO SUBMISSION OF A SEQUENCE LISTING AS A TEXT FILE

The Sequence Listing written in file 1306735 Sequence Listing.txt created on May 16, 2022, 113,550 bytes, machine format IBM-PC, MS-Windows operating system, is hereby incorporated by reference in its entirety for all purposes.

### BACKGROUND

One of the most prominent bottlenecks in the gene editing process is the ability to identify and isolate individual cells with desired edits within a population of treated cells. Current approaches typically require time-consuming and labor-intensive single cell isolation followed by population expansion (1-3), followed by destruction of some portion of an expanded cell population for downstream in vitro analysis of DNA sequence content (4-7). Although the gene editing validation issues have spurred novel solutions such as surface oligopeptide knock-in for rapid target selection by FACS sorting that does not rely on cell cloning (8), cell types that exhibit low efficiencies of transfection, editing, single cell isolation, or population expansion can be particularly challenging (9-13). To compound this problem, homology directed repair (HDR) can exhibit extremely low efficiency in certain cell types (14).

State-of-the-art molecular probes of specific DNA sequences in living cells have been used to tether fluorescent proteins such as green fluorescent protein (GFP) to DNA-binding proteins, including catalytically dead Cas9 (dCas9). Such probes have been widely used. However, an important property of such probes is that they are “always on”, meaning that it is impossible to distinguish between a probe bound to a target site from one floating free in the nucleus. For that reason, the use of such probes has been limited to regions containing tandemly repeated sequences or using 26-37 gRNAs, so that a high local concentration of fluorescence signal can be detected over the “always-on” background GFP fluorescence. Accordingly, such a system is not useful for detecting unique DNA edits.

There is therefore a need for new approaches that allow the detection of specific genomic sequences or modifications in, e.g., non-tandemly repeated sequences, including in cells with low rates of transfection, editing, isolation, or expansion. The present invention addresses this need and provides other advantages as well.

### BRIEF SUMMARY

In one aspect, the present invention provides a method of detecting the presence of a genomic sequence of interest in a living cell, the method comprising: i) introducing a first fusion protein into the cell, the first fusion protein comprising an RNA-guided nuclease fused to the large subunit of NanoLuc luciferase (LgBiT); ii) introducing a second fusion protein into the cell, the second fusion protein comprising an RNA-guided nuclease fused to the small subunit of NanoLuc luciferase (SmBiT); iii) introducing a first and a second guide RNA into the cell, wherein the first and the second guide RNA are complementary to a first and a second nucleotide sequence within the genomic sequence of interest such that, in the presence of the genomic sequence of interest, when the first guide RNA is bound by the first fusion protein and the second guide RNA is bound by the second fusion protein, the guide RNAs direct the binding of the fusion proteins to the genomic sequence of interest such that the LgBiT and SmBiT elements are in proximity and luminescence is produced, indicating the presence of the genomic sequence of interest in the cell.

Any RNA-guided nuclease can be used in the present methods, i.e., any nuclease that can bind to a guide RNA and be directed to a specific nucleotide sequence by the guide RNA. In some embodiments, the RNA-guided nuclease is a Cas nuclease such as Cas9 or Cpf1. In some embodiments of the method, the RNA-guided nuclease is nuclease dead, i.e., is capable of binding to but does not cleave the DNA. In a particular embodiment, the nuclease is dCas9. In the present methods, the nuclease is fused to a portion of the Nano-Luc (NLuc) luciferase. In particular embodiments, the fusion proteins comprise a large and a small fragment of the full-length Nano-Luc, i.e., LgBiT and SmBiT, respectively. Exemplary sequences of LgBiT and SmBiT can be seen, e.g., in Example 2 and in the fusion proteins shown as SEQ ID NOS:1-4, although derivatives and variants of the sequences can be used as well, so long that the two fragments can physically associate and produce luminescence. LgBiT and/or SmBiT can be fused at either the N- or C-terminus of the nuclease, e.g., dCas9, although it will be appreciated that the subunit is not necessarily fused directly to the terminus, as the fragment may be separated by the nuclease by, e.g., a spacer or linker element. In addition, the fusion protein may contain other sequence elements such as epitope tags, nuclear localization signals (NLS), etc. In particular embodiments, the first fusion protein is LgBiT-dCas9 (i.e., LgBiT fused at the N-terminus of dCas9), and the second fusion protein is dCas9-SmBiT (i.e., SmBiT fused at the C-terminus of dCas9). In particular embodiments, the first fusion protein comprises an amino acid sequence identical, or, e.g., at least about 80%, 85%, 90%, 95%, 96%, 97%, 98%, 99%, or more idential, to any of SEQ ID NOS: 1-4. In particular embodiments, the second fusion protein comprises an amino acid sequence identical, or, e.g., at least about 80%, 85%, 90%, 95%, 96%, 97%, 98%, 99%, or more idential, to any of SEQ ID NOS: 1-4.

Various methods can be used to introduce the fusion proteins and/or guide RNAs into the cell. In some embodiments, the fusion proteins and/or guide RNAs are introduced by introducing one or more polynucleotides encoding one or more fusion proteins or guide RNAs into the cell, such that the fusion proteins and/or guide RNA are expressed in the cell. The polynucleotides can be introduced, e.g., using a viral vector, or by transfecting naked DNA or RNA. In some embodiments, the polynucleotide comprises an expression cassette comprising a coding sequence encoding a fusion protein or guide RNA, operably linked to a promoter.

In some embodiments, the first guide RNA and the first fusion protein, and the second guide RNA and the second fusion protein, are first produced in vitro and assembled into ribonucleoproteins (RNPs), and the RNPs are then introduced into the cell, e.g., by lipofection or electroporation.

In some embodiments, luminescence is detected as relative fluorescence units (RFU) or relative luminescence units (RLU). RFU/RLU can be measured and calculated as described elsewhere herein, and the signal:noise ratio calculated, i.e., the ratio of the “signal” RFU/RLU in the presence of the fusion proteins, guide RNAs, and the genomic sequence targeted by the guide RNAs relative to the “noise” RFU/RLU in the absence of one or more of these elements. In some embodiments, the signal:noise ratio of the RFU/RLU in the presence of the first and second fusion proteins, the first and second guide RNAs, and the genomic sequence of interest relative to the RFU/RLU in the absence of any one or more of the first and second fusion proteins, the first and second guide RNAs, or the genomic sequence of interest is at least 2.5:1, 5:1, 10:1, 15:1, 20:1, 25:1, or more.

The two guide RNAs are designed to target, i.e., be complementary to, two distinct nucleotide sequences within the genome that are near to one another such that, when the two fusion proteins are directed to the target nucleotide sequences by the two guide RNAs, the fragments of the luminescent reporter, e.g., LgBiT and SmBiT, within the fusion proteins can physically interact and produce luminescence. For example, in some embodiments, the two target nucleotide sequences are within 10, 20, 30, 40, or 50 nucleotides of one another. The two target nucleotide sequences can be in any directional relationship on the target locus, i.e., they can be present in tandem, in inversed orientation, or in everted orientation relative to one another. In some embodiments of the method, the first and second nucleotide sequences are arrayed in tandem and are present within 50 nucleotides of one another. In some embodiments, the first and second nucleotide sequences are arrayed in inverse orientation and are present within 50 nucleotides of one another. In some embodiments, the first and second nucleotide sequences are arrayed in everted orientation and are present within 50 nucleotides of one another. In one embodiment, the first and second nucleotide sequences are arranged in tandem and are 40-bp apart. In one embodiment, the first and second nucleotide sequences are arranged in inverted orientation and are 7-bp apart. Any sequences can be selected for targeting by the guide RNAs, provided that they are each adjacent to a PAM sequence, including sequences that are only present once or a small number of times in the genome (i.e., that are not tandemly repeated sequences).

In some embodiments, the methods are performed with a fusion protein comprising a protein or protein domain that is sensitive to an epigenetic modification such as 5-methyl-C. For example, MBD2, which binds to 5-methyl-C, can be used. In some such embodiments, the methods are performed with fusion proteins comprising a protein or fragment thereof that is sensitive to an epigenetic modification, comprising LgBiT or SmBiT, and comprising an RNA-guided nuclease or fragment thereof, wherein the DNA binding domain of the nuclease has been replaced with the epigenetic modification-sensitive protein. For example, the guide RNAs could direct the fusion proteins to a genomic site such as a promoter that potentially comprises an epigenetic modification such as 5-methyl-C, and the detection of a luminescent signal can indicate the presence of methylation at the promoter. In some embodiments, one of the fusion proteins comprises the sequence shown as SEQ ID NO:1 or a fragment thereof, or a sequence comprising at least about 80%, 85%, 90%, 95%, 96%, 97%, 98%, 99%, or more identity to SEQ ID NO:1 or a fragment thereof. In some embodiments, one of the fusion proteins comprises the sequence shown as SEQ ID NO:2 or a fragment thereof, or a sequence comprising at least about 80%, 85%, 90%, 95%, 96%, 97%, 98%, 99%, or more identity to SEQ ID NO:2 or a fragment thereof. In some embodiments, one of the fusion proteins comprises the sequence shown as SEQ ID NO:3 or a fragment thereof, or a sequence comprising at least about 80%, 85%, 90%, 95%, 96%, 97%, 98%, 99%, or more identity to SEQ ID NO:3 or a fragment thereof. In some embodiments, one of the fusion proteins comprises the sequence shown as SEQ ID NO:4 or a fragment thereof, or a sequence comprising at least about 80%, 85%, 90%, 95%, 96%, 97%, 98%, 99%, or more identity to SEQ ID NO:4 or a fragment thereof.

The present methods can be used for a variety of applications. For example, in some embodiments, the methods are used to detect a genomic modification induced by CRISPR-Cas in the cell. For example, the genomic sequence of interest that is detected using the methods can correspond to a sequence that is only present following a CRISPR-Cas-mediated modification. In this way, cells can be identified that have successfully been modified and can therefore be distinguished from unmodified cells. In some embodiments, the cell is part of a population of cells, and the method is used to detect individual cells within the population that have undergone the genomic modification. The methods can also be used to identify modifications that are induced independently of CRISPR-Cas, e.g., spontaneous mutations or mutations induced by other genomic editing methods. The methods can also be used to identify specific polymorphisms in an individual or population.

The two fusion proteins can be introduced into the cell in any relative amount. For example, in some embodiments equal amounts of the two fusion proteins are introduced. In some embodiments, a greater amount of one of the fusion proteins is introduced. In some embodiments of the method, the second fusion protein, i.e., the fusion protein comprising SmBiT, is introduced at a molar excess relative to the first fusion protein, i.e. the fusion protein comprising LgBiT. In some embodiments, the molar excess is from 5:1 to 15:1. In some embodiments, the molar excess is 10:1.

In some embodiments of the method, the cell is a eukaryotic cell. In some embodiments, the eukaryotic cell is a mammalian cell. In some embodiments, the mammalian cell is a human cell. In some embodiments, the cell is of a type, or is modified using a procedure, that is associated with a low frequency of transfection, successful gene editing, isolation, or expansion, such as a primary cell or a stem cell undergoing homology directed repair (HDR).

The present disclosure also provides fusion proteins and guide RNAs, polynucleotides encoding the fusion proteins and guide RNAs, expression cassettes or vectors comprising the polynucleotides, as well as cells comprising any of the herein-described fusion proteins, guide RNAs, expression cassettes, polynucleotides, or vectors. For example, in another aspect, the present disclosure provides a cell comprising: i) a first fusion protein comprising an RNA-guided nuclease fused to LgBiT; ii) a second fusion protein comprising an RNA-guided nuclease fused to SmBiT; iii) a first guide RNA that is complementary to a first nucleotide sequence within the genome and that can be bound by the first fusion protein and direct it to the first nucleotide sequence; and iv) a second guide RNA that is complementary to a second nucleotide sequence within the genome and that can be bound by the second fusion protein and direct it to the second nucleotide sequence; wherein the first and the second nucleotide sequences are arranged in the genome such that when the first and second fusion proteins are directed to the first and second nucleotide sequences by the first and second guide RNAs, the LgBiT and SmBiT elements of the fusion proteins are brought into in proximity and luminescence is produced. In some embodiments, the method is used to detect a genomic editing event (e.g., CRISPR-mediated editing) in the cell. In some embodiments, the method is used to detect a mutation in the cell.

In some embodiments, the RNA-guided nuclease is dCas9. In some embodiments, the first fusion protein is LgBiT-dCas9. In some embodiments, the second fusion protein is dCas9-SmBiT. In some embodiments, the RNA-guided nuclease is Cpf1. In some embodiments, the fusion proteins comprise a protein that binds selectively to an epigenetic modification, or an absence thereof. For example, in some embodiments the fusion protein comprises MBD2 or a fragment or derivative thereof.

In some embodiments, the first and second nucleotide sequences are arrayed in tandem and are present within 50 nucleotides of one another. In some embodiments, the first and second nucleotide sequences are arrayed in inverse orientation and are present within 50 nucleotides of one another. In some embodiments, the first and second nucleotide sequences are arrayed in everted orientation and are present within 50 nucleotides of one another. In some embodiments, the first and second nucleotide sequences are found within a genomic location, e.g., a promoter, that is potentially subject to an epigenetic modification, such as 5-methyl-C.

In some embodiments, the first and second fusion protein are present in approximately equal amounts. In some embodiments, one of the fusion proteins is present at a higher level than the other fusion protein. In some embodiments, the second fusion protein is present at a molar excess relative to the first fusion protein. In some embodiments, the molar excess is from 5:1 to 15:1. In some embodiments, the molar excess is 10:1.

In some embodiments, the cell is a eukaryotic cell. In some embodiments, the cell is a mammalian cell. In some embodiments, the cell is a human cell. In some embodiments, the cell is a primary cell. In some embodiments, the cell is a stem cell. In some embodiments, the cell has been modified by HDR, e.g., in conjunction with cleavage by a CRISPR-Cas nuclease.

### BRIEF DESCRIPTION OF THE DRAWINGS

FIGS. 1A-1D. FIG. 1A: A cartoon depiction of sequence-dependent reconstitution of NanoLuc luciferase. FIG. 1B: Cartoon representation of dCas9-NanoBiT and full-length dCas9-NanoLuc fusion constructs. FIG. 1C: Schematic of target site designs with PAM sites in tandem (parallel on the same strand), inverted (PAMs oriented inward on opposite strands) and everted (PAMs oriented outward on opposite strands) (SEQ ID NOS 16, 18, 16, 128, 128, and 16, respectively, in order of appearance). FIG. 1D: A heat map showing variation in signal intensity between four possible orientations of dCas9-NanoBiT fusion proteins across 33 DNA target site spacings and orientations. Sequential scale ranges from lowest signals of the set (magenta) to highest signals of the set (green).

FIGS. 2A-2C. FIG. 2A: 12 target sequence scaffolds tested in live cells using the RNP delivery method. In each condition, dCas9-SmBiT was complexed with IVT gRNA for the upstream target site and LgBiT-dCas9 was complexed with IVT gRNA for the downstream target site and delivered to HEK 293T cells. FIG. 2B: Effect of decreasing target sequence scaffold concentration on NLuc signal intensity using RNP-based delivery of biosensor components to live cells. FIG. 2C: A comparison of dimeric DNA biosensor function across six different cell lines. Apparent signal-to-noise ratios in FIGS. 2A-2B (comparisons made to no DNA background conditions) are listed in parentheses above each biosensing condition. Data in FIGS. 2A-2B are presented as the mean±s.e.m., n=3, where n represents the number of independent experimental technical replicates included in parallel; unpaired two-sided Student's t-test, *P<0.05; **P<0.01; ***P<0.001; ****P<0.0001.

FIGS. 3A-3K. FIGS. 3A-3E: GFP, NLuc, and merged images taken on the Leica DM6000 B upright microscope at 10× magnification. GFP images were taken with 150 ms exposure to excitation light. NLuc images were taken with 30s exposure and gain of 2.0 in a dark box. RNP constructs and DNA target site scaffolds delivered are shown above image sets. Scale bars=50 μM. FIG. 3F: A bioluminescence image taken on the IVIS Spectrum Bioluminescence Imaging System of live HEK 293T cells expressing the same RNPs as before with delivery of the tandem target sites 10 bp apart scaffold. Signal scaling shown at right. FIG. 3G: A bioluminescence image taken on the IVIS Spectrum Bioluminescence Imaging System of live HEK 293T cells expressing the same RNPs as before with delivery of the inverted target sites 15 bp apart scaffold. Signal scaling shown at right. FIG. 3H: A bioluminescence image taken on the IVIS Spectrum Bioluminescence Imaging System of live HEK 293T cells expressing the same RNPs as before without target DNA. Signal scaling shown at right. FIG. 3I: A bioluminescence image taken on the IVIS Spectrum Bioluminescence Imaging System of live HEK 293T cells expressing the LgBiT-dCas9 fusion construct alone. Signal scaling shown at right. FIG. 3J: A bioluminescence image taken on the IVIS Spectrum Bioluminescence Imaging System of live HEK 293T cells expressing the NLuc-dCas9 fusion construct alone. Signal scaling shown at right. FIG. 3K: Quantification of cell region ROIs for various transfection conditions in IVIS Spectrum LivingImage software. Apparent signal-to-noise ratios (comparisons made to no DNA background condition) are listed in parentheses above each biosensing condition. Data in FIG. 3K is presented as the mean±s.e.m., n=20, where n represents the number of independent experimental technical replicates included in parallel; unpaired two-sided Student's t-test, *P<0.05; **P<0.01; ***P<0.001; ****P<0.0001.

FIGS. 4A-4I. FIG. 4A: Cartoon visualization of the repetitive and non-repetitive regions of the human MUC4 locus. FIG. 4A discloses SEQ ID NO: 129. FIG. 4B: dCas9-NanoBiT biosensing of the repetitive region of MUC4 exon 2 in live HeLa cells. FIG. 4C: dCas9-NanoBiT biosensing of the non-repetitive region of MUC4 intron 1 in live HeLa cells. FIG. 4D: dCas9-NanoBiT biosensing of the repetitive region of MUC4 exon 2 in live HEK 293T cells. FIG. 4E: dCas9-NanoBiT biosensing of the non-repetitive region of MUC4 intron 1 in live HEK 293T cells. FIG. 4F: Signal quantification of the dimeric probe binding the repetitive region of MUC4 exon 2 in live HeLa cells. FIG. 4G: Signal quantification of the dimeric probe binding the non-repetitive region of MUC4 intron 1 in live HeLa cells. Error bars represent s.e.m., n=5. FIG. 4H: Signal quantification of the dimeric probe binding the repetitive region of MUC4 exon 2 in live HEK 293T cells. FIG. 4I: Signal quantification of the dimeric probe binding the non-repetitive region of MUC4 intron 1 in live HEK 293T cells. Apparent signal-to-noise ratios in FIGS. 4F-4I (comparisons made to no sgRNA background conditions) are listed in parentheses above each biosensing condition. Data in FIGS. 4F, 4H, and 4I are presented as the mean±s.e.m., n=3, where n represents the number of independent experimental technical replicates included in parallel; unpaired two-sided Student's t-test, *P<0.05; **P<0.01; ***P<0.001; ****P<0.0001.

FIGS. 5A-5D. FIG. 5A: Cartoon visualization of the editing experiments conducted at the human 8q24 cancer risk and PALB2 loci. gRNAs used for editing are shown in blue and gRNAs around the site of mutation that were used for detection of mutant cells in biosensing experiments are shown in red. Single base pair edits are shown in bold. FIG. 5A discloses SEQ ID NOS 130-131, 133, 123, 130, 132 and 134-135, respectively, in order of appearance. FIG. 5B: Bioluminescence images taken on the IVIS Spectrum Bioluminescence Imaging System of the dimeric DNA biosensor applied to the PALB2 locus after targeted CRISPR-Cas9 genome editing. Wild type HEK 293 cells expressing the LgBiT-dCas9 and dCas9-SmBiT protein constructs and several gRNAs are compared to HEK 293 cells homozygous for a G->T missense mutation at the PALB2 locus expressing the same biosensor components and gRNAs. Both wild type and mutant biosensing conditions are compared to a background condition where the biosensor components are not directed to bind the DNA by gRNAs. FIG. 5C: Signal differences in directed probe binding conditions compared to background conditions for both G->T mutant and wild type HEK 293 cells. FIG. 5D: Application of the dimeric DNA biosensor with LgBiT-dCas9 and dCas9-SmBiT to the 8q24 risk locus after targeted CRISPR-Cas9 genome editing. Signal differences in directed probe binding conditions are compared to background conditions for both G->T homozygous mutant and wild type HCT116 cells. Apparent signal-to-noise ratios in FIGS. 5C-5D (comparisons made to no sgRNA background conditions) are listed in parentheses above each biosensing condition. Data in c-d are presented as the mean±s.e.m., n=5, where n represents the number of independent experimental technical replicates included in parallel; unpaired two-sided Student's t-test, *P<0.05; **P<0.01; ***P<0.001; ****P<0.0001.

FIGS. 6A-6E: Optimization of plasmid-based delivery. FIG. 6A: Relative NLuc signal intensity across indicated molar transfection ratios of LgBiT-dCas9 to dCas9-SmBiT with (blue bars) or without (red bars) DNA target plasmids in HEK 293T cells. FIG. 6B: Signal intensities of tandem 40-bp and inverted 7-bp DNA targets compared to no DNA controls over 1:1, 1:1.2, 1:2, 1:5, 1:10, and 1:20 fusion protein:gRNA molar transfection ratios. FIG. 6C: Relative signal intensities using targets of indicated spacing and orientation. gRNAs plasmids were transfected at 20-fold molar excess to dCas9-NanoBiT fusion constructs. FIG. 6D: The dependence of target plasmid concentration was assayed using fixed ratios of the dCas9-NanoBiT and gRNA plasmids. FIG. 6E: The dependence of incubation time post-transfection was assayed using fixed ratios of all plasmids in the indicated configurations. Apparent signal-to-noise ratios in a-e (comparisons made to no DNA background conditions) are listed in parentheses above each biosensing condition. Data in FIGS. 6A-6E are presented as the mean±s.e.m., n=3, where n represents the number of independent experimental technical replicates included in parallel; unpaired two-sided Student's t-test, *P<0.05; **P<0.01; ***P<0.001; ****P<0.0001.

FIGS. 7A-7D: Optimization of RNP-based delivery. FIG. 7A: Initial data showing relative luminescent signals immediately after complexation of LgBiT-dCas9 and dCas9-SmBiT RNPs. FIGS. 7B-7C: Time course experiments showing luminescent signal decay when LgBiT-dCas9 and dCas9-SmBiT RNPs bind tandem 40-bp (blue line) and inverted 7-bp (red line) target DNA plasmids in vitro. FIG. 7D: Initial experiments showing RNP delivery of biosensor components to live HEK 293T cells. In each condition, dCas9-SmBiT-C was complexed with IVT gRNA for the upstream target site and LgBiT-N-dCas9 was complexed with IVT gRNA for the downstream target site. Apparent signal-to-noise ratios in FIGS. 7A and 7D (comparisons made to no DNA background conditions) are listed in parentheses above each biosensing condition. Data in FIGS. 7A and 7D are presented as the mean±s.e.m., n=3, where n represents the number of independent experimental technical replicates included in parallel; unpaired two-sided Student's 1-test, P<0.05; **P<0.01; ***P<0,001; ****P<0.0001.

FIG. 8: IVIS GFP Images. IVIS GFP images used for normalization of images shown in FIGS. 3F-3J.

FIGS. 9A-9B: Signal-to-noise of monomeric probes. FIG. 9A: Signal compared to background for monomeric dCas9-EGFP fluorescent probe shown in two cell lines. FIG. 9B: Signal compared to background for monomeric NLuc-dCas9 luminescent probe shown in two cell lines. Apparent signal-to-noise ratios in FIGS. 9A-9B (comparisons made to no sgRNA background conditions) are listed in parentheses above each probe's biosensing condition. Data in FIGS. 9A-9B are presented as the mean±s.e.m., n=5, where n represents the number of independent experimental technical replicates included in parallel; unpaired two-sided Student's t-test, *P<0.05; **P<0.01; ***P<0.001; ****P<0.0001.

FIG. 10: Biosensor signal output variability across seven individual non-repetitive loci at MUC4. Signal intensities from a DNA biosensing experiment where four orientations of dCas9-NanoBiT RNPs were directed to bind seven individual locations within the non-repetitive region of the human MUC4 gene. Apparent signal-to-noise ratios (comparisons made to no sgRNA background conditions separately for each fusion protein orientation) are listed in parentheses above each biosensing condition. Data is presented as the mean±s.e.m., n=5, where n represents the number of independent experimental technical replicates included in parallel; unpaired two-sided Student's t-test, *P<0.05; **P<0.01; ***P<0.001; ****P<0.0001.

FIG. 11: HC91V3 (iCas9V3) vector map.

FIG. 12. Top: Western Blot for HA epitope tagged proteins. Left to right: SmBiT-dCas9, LgBiT-dCas9, NLuc-dCas9. Bottom: Western Blot for 3×-Flag epitope tagged proteins. Left to right: dCas9-SmBiT, dCas9-LgBiT.

FIG. 13: dCas9-NanoBiT biosensing of four loci within the repetitive region of exon 2 of the MUC4 gene in HEK 293T cells. Control conditions representing transfections of probe without gRNA and transfections of each binding partner of the probe alone are shown. Error bars represent s.e.m., 8<n<82.

FIG. 14: dCas9-NanoBiT biosensing images of three loci individually and in combinations of two and three within the nonrepetitive region of intron 1 of the MUC4 gene in six human cell lines. Controls with no gRNA transfected, LgBiT-dCas9 only transfected, and NLuc-dCas9 probe transfected are shown for comparison. Images represent merged GFP and NLuc channels at 10× magnification on the Leica DM6000B upright microscope.

FIGS. 15AS-15F: dCas9-NanoBiT biosensing of three loci individually and in combinations of two and three within the nonrepetive region of intron 1 of the MUC4 gene in six human cell lines (HEK 293T, FIG. 15A; HeLa, FIG. 15B; MCF7, FIG. 15C; HCT116, FIG. 15D; K562, FIG. 15E; That, FIG. 15F). Control conditions without gRNA and without target DNA (mouse cell lines transfected with gRNA to locus 1) were included as auto-association noise measurements for the probe. Additional negative control transfections of each binding partner in the dimeric probe alone were also included. A positive control condition where the same molar quantity of full-length NLuc dCas9 monomeric biosensor was transfected was also included. Error bars represent s.e.m., 10<n<439.

FIGS. 16A-16F: ROC curve analysis of single locus detection (Locus 1) in six cell types (HEK 293T, FIG. 16A; HeLa, FIG. 16B; MCF7, FIG. 16C; HCT116, FIG. 16D; K562, FIG. 16E; JLat, FIG. 16F). False positives were determined by signals due to auto-assembly (No sgRNA). Even in cells for which auto-assembly was high compared to true positives, area under the curve is>0.84 for all cell types, and>0.93 for most cell types.

FIGS. 17A-17B: dCas9-NanoBiT biosensing of locus 1 within the nonrepetitive region of intron 1 of MUC4 in 2 human cell lines. (HeLa, FIG. 17A; MCF7, FIG. 17B) Total molar quantity of dCas9-NanoBiT probe was reduced 10-fold and 100-fold compared to the data shown in FIGS. 15A-15F. Control conditions without gRNA, with transfections of each binding partner of the probe alone, and with the full-length NLuc-dCas9 probe are shown. Error bars represent s.e.m., 5<n<167.

### DETAILED DESCRIPTION OF THE INVENTION

### 1. Introduction

The present invention provides the first split-enzyme system that can detect specific DNA sequences in living cells. With the advent of CRISPR/Cas9, the primary bottleneck in gene editing is no longer the nuclease. Among the remaining challenges is the ability to identify and isolate cells in which the desired genetic or epigenetic events have occurred. This is of particular concern for cell types or procedures in which the frequency of successful gene edits is low, such as homology directed repair (HDR) in primary cells and stem cells. Indeed, a considerable portion of the time required for gene editing is often the isolation of cells with the desired genotype.

The present disclosure provides a split-enzyme system based on, e.g., luciferase, linked to programmable DNA-binding domains can detect genetic information in living cells. Building on the Nano-Luc systems, we have constructed a split-luciferase system linked to dCas9 programmable DNA-binding domains. The present split-luciferase reporter system can detect the presence of a target genetic sequence at, e.g., 10-fold above background in living cells. To date, no such system has been used in live cells.

In addition to DNA sequences such as gene edits, in some embodiments the DNA-binding domain of the nuclease is replaced by a protein that “reads” epigenetic information, such as binding of MBD2 to 5-methyl-C, thereby allowing the use of probes that could read epigenetic information.

The present methods and compositions provide a “turn-on” probe, which can remain “off” until bound to its target site. The use of a split-enzyme, such as split luciferase, adds catalytic amplification to the signal and can improve detection over 1,000-fold over non-enzymatic reporters such as GFP. The probes can be applied, e.g., to pools of treated cells, and then long-exposure light microscopy can be used to visualize cells that contain the correct target DNA sequence.

### 2. Definitions

As used herein, the following terms have the meanings ascribed to them unless specified otherwise.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the protein” includes reference to one or more proteins known to those skilled in the art, and so forth.

The terms “about” and “approximately” as used herein shall generally mean an acceptable degree of error for the quantity measured given the nature or precision of the measurements. Typically, exemplary degrees of error are within 20 percent (%), preferably within 10%, and more preferably within 5% of a given value or range of values. Any reference to “about X” specifically indicates at least the values X, 0.8X, 0.81X, 0.82X, 0.83X, 0.84X, 0.85X, 0.86X, 0.87X, 0.88X, 0.89X, 0.9X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, 1.1X, 1.11X, 1.12X, 1.13X, 1.14X, 1.15X, 1.16X, 1.17X, 1.18X, 1.19X, and 1.2X. Thus, “about X” is intended to teach and provide written description support for a claim limitation of, e.g., “0.98X.”

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

“NanoLuc,” or NLuc, refers to luciferase system developed from a 19 kDa luciferase from the deep-sea shrimp Oplophorus gracilirostris and using the imidazopyrazinone furimazine as a substrate. See, e.g., Hall et al. (2012) ACS Chem Biol. 7(11): 1848-1857; England et al. (2016) Bioconjug Chem 27(5): 1175-1187, the entire disclosures of which are herein incorporated by reference. The sequence of full-length NanoLuc can be found, e.g., in Example 2, and NanoLuc enzymes and substrates can be obtained, e.g., from Promega. “LgBiT” and “SmBiT” refer to two independently optimized fragments of NLuc, which can physically interact and generate luminescence when present in proximity, e.g., when present within fusion proteins bound adjacently on genomic DNA, but which show minimal non-specific auto-association (and luminescence) when not bound to genomic DNA. Exemplary sequences of fusion proteins comprising LgBiT or SmBiT are shown, e.g., in SEQ ID NOS: 1-4, but it will be appreciated that variants of these sequences that are still capable of associating and producing a luminescent signal when present within fusion proteins as described herein can also be used.

The “CRISPR-Cas” system refers to a class of bacterial systems for defense against foreign nucleic acid. CRISPR-Cas systems are found in a wide range of eubacterial and archaeal organisms. CRISPR-Cas systems include type I, II, III, V, and VI sub-types. Wild-type type II CRISPR-Cas systems utilize the RNA-mediated nuclease, Cas9 in complex with guide and activating RNA to recognize and cleave foreign nucleic acid.

Cas9 homologs are found in a wide variety of eubacteria, including, but not limited to bacteria of the following taxonomic groups: Actinobacteria, Aquificae, Bacteroidetes-Chlorobi, Chlamydiae-Verrucomicrobia, Chlroflexi, Cyanobacteria, Firmicutes, Proteobacteria, Spirochaetes, and Thermotogae. An exemplary Cas9 polypeptide is the Streptococcus pyogenes Cas9 polypeptide (SpyCas9). Additional Cas9 proteins and homologs thereof are described in, e.g., Chylinksi, et al., RNA Biol. 2013 May 1; 10(5): 726-737 ; Nat. Rev. Microbiol. 2011 Jun.; 9(6): 467-477; Hou, et al., Proc Natl Acad Sci USA (2013) Sep. 24;110(39):15644-9; Sampson et al., Nature. 2013 May 9; 497(7448):254-7; and Jinek, et al., Science. 2012 Aug. 17; 337(6096):816-21. Cpf1 is a class II RNA-guided nuclease, as found in, e.g., Prevotella and Francisella bacteria. The RNA-guided nuclease can be nuclease defective. For example, the nuclease can be a nicking endonuclease that nicks target DNA, but does not cause double strand breakage. Cas9, for example, can also have both nuclease domains deactivated to generate “dead Cas9” (dCas9), a programmable DNA-binding protein with no nuclease activity.

A guide RNA, or gRNA or sgRNA, refers to an RNA molecule that can bind to a Cas nuclease, e.g., Cas9 or Cpf1, and that also comprises a spacer sequence, e.g., a 19 or 20 nucleotide sequence, that is complementary to a target sequence of interest. The guide RNA can bind to Cas9 or Cpf1 and direct it to the target sequence, thereby bringing about, e.g., the cleavage of the target sequence (with nuclease active Cas9 or Cpf1), or the binding of a catalytically dead nuclease such as dCas9. The target sequence of the guide RNA can be any unique sequence in the genome, provided that it is adjacent to a Protospacer Adjacent Motif (PAM). In the present methods, the target sequences of the two guide RNAs are selected such that their target sequences are close to each other in the genome, e.g., within 50 nucleotides of one another, such that the binding of the two fusion proteins comprising SmBiT and LgBiT to the two target sites allows the interaction of the SmBiT and LgBiT fragments of NLuc and the production of luminescence.

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. The promoter can be a heterologous promoter.

An “expression cassette” is a nucleic acid construct, generated recombinantly or synthetically, with a series of specified nucleic acid elements that permit transcription of a particular polynucleotide sequence in a host cell. An expression cassette may be part of a plasmid, viral genome, or nucleic acid fragment. Typically, an expression cassette includes a polynucleotide to be transcribed, operably linked to a promoter. The promoter can be a heterologous promoter. In the context of promoters operably linked to a polynucleotide, a “heterologous promoter” refers to a promoter that would not be so operably linked to the same polynucleotide as found in a product of nature (e.g., in a wild-type organism).

“Polypeptide,” “peptide,” and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

“Conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. In some cases, conservatively modified variants of Cas9 or sgRNA can have an increased stability, assembly, or activity as described herein.

As used in herein, the terms “identical” or percent “identity,” in the context of describing two or more polynucleotide or amino acid sequences, refer to two or more sequences or specified subsequences that are the same. Two sequences that are “substantially identical” have at least 60% identity, preferably 65%, 70%, 75%, 80%, 85%, 90%, 91%, 92%, 93, 94%, 95%, 96%, 97%, 98%, 99%, or 100% identity, when compared and aligned for maximum correspondence over a comparison window, or designated region as measured using a sequence comparison algorithm or by manual alignment and visual inspection where a specific region is not designated. With regard to polynucleotide sequences, this definition also refers to the complement of a test sequence. With regard to amino acid sequences, in some cases, the identity exists over a region that is at least about 50 amino acids or nucleotides in length, or more preferably over a region that is 75-100 amino acids or nucleotides in length.

### 3. Fusion proteins

The present methods and compositions involve the use of fusion proteins comprising an RNA-guided nuclease and a portion of a biosensor molecule, e.g., a bioluminescent protein sensor such as NLuc. The signal produced by the two portions or fragments of the biosensor when apart is low or absent, but a substantial signal is produced when the two portions are brought into proximity on a target sequence. In particular embodiments, increases in luminescence (e.g., RFU/RLU) of, e.g., 2.5, 5, 7.5, 10, 12.5, 15, 17.5, 20 fold or more, e.g., the signal detected in the presence of the fusion proteins, guide RNAs, and target DNA vs. the signal in the presence of the fusion proteins and guide RNAs, but without the target DNA (or with the fusion proteins but without the guide RNAs), are obtained using the present methods and compositions. In particular embodiments, the two fragments only weakly associate with each other (e.g., with a dissociation constant of 190 μM or higher), such that they must be brought into close proximity in order to recreate the full-length reporter and generate a substantial signal.

In some embodiments, any luminescent reporter, e.g., a bioluminescent or fluorescent biosensor, can be used, so long that the reporter can be separated into two (or more) fragments, wherein there is a substantial (e.g., 2, 3, 4, 5, 10, 15, 20 or more fold) increase in signal produced when the fragments are brought into proximity as compared to when they are apart. In some embodiments, a fluorescent reporter is used such as, GFP, RFP, EGFP, Emerald, Azami Green, mWasabi, ZsGreen, T-Sapphire, EBFP, Azurite, ECFP, Cerulean, mTurquoise, CyPet, AmCyanl, Midori-Ishi Cyan, mTFP1, EYFP, Topaz, Venus, Citrine, mBanana, mOrange, dTomato, mCherry, DsRed, mTangerine, mRuby, mApple, mStrawberry, mRaspberry, mPlum, or others.

In particular embodiments, the reporter is a bioluminescent reporter. In particular embodiments, the bioluminescent reporter is a luciferase-based reporter such as NanoLuc (NLuc) Luciferase, Firefly Luciferase, or Renilla Luciferase. In particular embodiments, the reporter used is NLuc (see, e.g., Hall et al. (2012) ACS Chemical Biology 7:1848-1857; England et al. (2016) Bioconjugate Chemistry 27:1175-1187; the entire disclosures of which are herein incorporated by reference). In particular embodiments, the fragments comprise or are derived from the NanoBiT (NanoLuc Binary Technology) complementation reporter system, comprising the subunits LgBiT (e.g., 18 kDa) and SmBiT (e.g., 1.3 kDa) (see, e.g., Dixon et al. (2016) ACS Chemical Biology 11:400-408, the entire disclosure of which is herein incorporated by reference). Exemplary sequences of LgBiT and SmBiT are presented, e.g., in Example 2 and within the fusion proteins of SEQ ID NOS:1-4, although derivatives, fragments, and variants of these sequences can be used as well (e.g., sequences comprising at least about 80%, 85%, 90%, 95%, 96%, 97%, 98%, 99% or more identity to the sequences shown in Example 2 or to all or part of any of SEQ ID NOS:1-4), so long that the two reporter fragments do not substantially intrinsically associate and do not produce substantial luminescence when apart, but they produce a substantial increase in luminescence when brought into close proximity, e.g., using the present methods.

In addition to the luminescent components, the fusion proteins of the present disclosure comprise RNA-guided nucleases. For example, each of the two components of the system comprises a fragment of a luminescent reporter and an RNA-binding protein. Any RNA-guided nuclease can be used in the present methods, i.e., any nuclease that can bind to a guide RNA and be directed to a specific nucleotide sequence by the guide RNA. In some embodiments, the RNA-guided nuclease is a Cas nuclease such as Cas9 or Cpf1. In particular embodiments, the RNA-guided nuclease is nuclease dead, i.e., is capable of binding to but does not cleave the DNA. In a particular embodiment, the nuclease is dCas9.

In addition to the CRISPR/Cas9 platform (which is a type II CRISPR/Cas system), alternative systems exist including type I CRISPR/Cas systems, type III CRISPR/Cas systems, and type V CRISPR/Cas systems. Various CRISPR/Cas9 systems have been disclosed, including Streptococcus pyogenes Cas9 (SpCas9), Streptococcus thermophilus Cas9 (StCas9), Campylobacter jejuni Cas9 (CjCas9) and Neisseria cinerea Cas9 (NcCas9) to name a few. In particular embodiments, the Cas9 is from Streptococcus pyogenes. Alternatives to the Cas system include the Francisella novicida Cpf1 (FnCpf1), Acidaminococcus sp. Cpf1 (AsCpf1), and Lachnospiraceae bacterium ND2006 Cpf1 (LbCpf1) systems. Any of the above CRISPR systems may be used in the herein-disclosed methods.

Each of the two fragments of the reporter, e.g., LgBiT and SmBiT, can be fused at either the N- or C-terminus of the nuclease, e.g., dCas9. In some embodiments, LgBiT is used and is fused to the N-terminus of the nuclease. In some embodiments, LgBiT is used and is fused to the C-terminus of the nuclease. In some embodiments, SmBiT is used and is fused to the N-terminus of the nuclease. In some embodiments, SmBiT is used and is fused to the C-terminus of the nuclease. In particular embodiments, the first fusion protein is LgBiT-dCas9 (i.e., LgBiT fused at the N-terminus of dCas9), and the second fusion protein is dCas9-SmBiT (i.e., SmBiT fused at the C-terminus of dCas9). In some embodiments, one of the fusion proteins comprises the sequence shown as SEQ ID NO:1 or SEQ ID NO:3, or a sequence comprising at least about 80%, 85%, 90%, 95%, 96%, 97%, 98%, 99%, or more identity to SEQ ID NO:1 or SEQ ID NO:3, and the other fusion protein comprises the sequence shown as SEQ ID NO:2 or SEQ ID NO:4, or a sequence comprising at least about 80%, 85%, 90%, 95%, 96%, 97%, 98%, 99%, or more identity to SEQ ID NO:2 or SEQ ID NO:4.

In some embodiments, the fusion protein comprises one or more linker elements, e.g., a (GGS)5 flexible linker (SEQ ID NO: 5), e.g., between the nuclease and the luminescent reporter fragment within the fusion protein. In addition, the fusion protein may contain other sequence elements such as epitope tags (e.g., an HA tag), nuclear localization signals (NLS), or other elements.

In some embodiments, the fusion protein comprises a protein or protein domain that is sensitive to an epigenetic modification such as 5-methyl-C. For example, in some embodiments MBD2 (see, e.g., UniProt ID Q9UBB5, or NCBI Gene ID 8932), which binds to 5-methyl-C, can be used. In some such embodiments, the methods are performed with fusion proteins comprising a protein or fragment thereof that is sensitive to an epigenetic modification, comprising LgBiT or SmBiT, and comprising an RNA-guided nuclease or fragment thereof, wherein the DNA binding domain of the nuclease has been replaced with the epigenetic modification-sensitive protein. For example, the guide RNAs could direct the fusion proteins to a genomic site such as a promoter that potentially comprises an epigenetic modification such as 5-methyl-C, and the detection of a luminescent signal can indicate the presence of methylation at the promoter.

In some embodiments, the fusion proteins are produced recombinantly, e.g., polynucleotides encoding the fusion proteins are introduced into host cells, e.g., bacterial host cells, and the cells grown under conditions conducive to the expression of the protein, which can then be purified using standard methds and then introduced into the cells (e.g., as RNPs with guide RNAs) in which a genomic modification is potentially detected using the present methods. In some embodiments, polynucleotides encoding the fusion proteins, e.g., within a vector, are introduced directly into the cells in which a genomic modification may be detected, such that the fusion proteins are expressed directly in the cells.

### 4. Guide RNAs

The guide RNAs (e.g., single guide RNAs, or sgRNAs) of the present disclosure are used as pairs of guide RNAs that target two sequences in close proximity to one another in the genome (or on a plasmid). Guide RNAs, e.g., sgRNAs, interact with a site-directed nuclease such as Cas9 and specifically bind to or hybridize to a target nucleic acid within the genome of a cell, such that the sgRNA and the site-directed nuclease co-localize to the target nucleic acid in the genome of the cell. Accordingly, using the present guide RNAs, one guide RNA will bind to one fusion protein (e.g., comprising LgBiT) and the other guide RNA will bind to the other fusion protein (e.g., comprising SmBiT), such that the two fusion proteins will be brought into close proximity when they bind the adjacent targeted DNA sequences. In particular embodiments, a single guide RNA, or sgRNA, is used. sgRNAs as used herein comprise a targeting sequence (of, e.g., 18-25 nucleotides, or 18, 19, 20, 21, 22, 23, 24, or 25 nucleotides) comprising homology (or complementarity) to a target DNA sequence, and a constant region that mediates binding to Cas9 or another RNA-guided nuclease. The sgRNAs can target any sequences in close proximity to one another within a target that are adjacent to PAM sequences.

In some embodiments, the two target sequences of the guide RNAs are separated by, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more nucleotides. In some embodiments, the two target sequences are arranged in tandem orientation. In some embodiments, the two target sequences are arranged in inverted orientation relative to one another. In some embodiments, the two target sequences are arranged in everted orientation relative to one another. In some embodiments, the two target sequences are on the same strand of the DNA double helix. In some embodiments, the two target sequences are on different strands of the DNA double helix. In some embodiments, the two target sequences are in tandem and separated by, e.g., about 1, 10, 40, or 45 nucleotides. In some embodiments, the two target sequences are in inverted orientation and are separated by, e.g., about 7, 25, or 45 nucleotides. In some embodiments, the two target sequences are in inverted orientation and are separated by, e.g., about 30, 35, or 50 nucleotides. In particular embodiments, the two target sequences are in tandem and are separated by about 40 nucleotides, or are in inverted orientation and are separated by about 7 nucleotides.

In some embodiments, the present methods and compositions are used to detect specific sequences in a genome, e.g., a specific mutation genomic editing event. For example, a guide RNA can be used that detects a specific genomic sequence, e.g., a sequence that is potentially mutated, wherein the mutation would lead to a decrease in or loss of binding of the guide RNA and associated fusion protein and consequently a decrease in the luminescent signal, or a sequence that is acquired upon mutation or editing, wherein the mutation would lead to an increase in binding of the guide RNA and associated fusion protein, and consequently an increase in the luminescent signal in the cell. Such methods can be used, e.g., to detect individually edited cells, which could then be isolated for clonal expansion. The target sequence can be present in a repetitive or nonrepetitive region of the genome or within a locus.

In some embodiments, the guide RNAs (e.g., sgRNAs) comprise one or more modified nucleotides. For example, the polynucleotide sequences of the guide RNAs may also comprise RNA analogs, derivatives, or combinations thereof. For example, the probes can be modified at the base moiety, at the sugar moiety, or at the phosphate backbone (e.g., phosphorothioates). In some embodiments, the guide RNAs comprise 3′ phosphorothiate internucleotide linkages, 2′-O-methyl-3′-phosphoacetate modifications, 2′-fluoro-pyrimidines, S-constrained ethyl sugar modifications, or others, at one or more nucleotides. In particular embodiments, the guide RNAs comprise 2′-O-methyl-3′-phosphorothioate (MS) modifications at one or more nucleotides (see, e.g., Hendel et al. (2015) Nat. Biotech. 33(9):985-989, the entire disclosure of which is herein incorporated by reference). In particular embodiments, the 2′-O-methyl-3′-phosphorothioate (MS) modifications are at the three terminal nucleotides of the 5′ and 3′ ends of the guide RNA (e.g., sgRNA).

The guide RNAs can be obtained in any of a number of ways. For sgRNAs, primers can be synthesized in the laboratory using an oligo synthesizer, e.g., as sold by Applied Biosystems, Biolytic Lab Performance, Sierra Biosystems, or others. Alternatively, primers and probes with any desired sequence and/or modification can be readily ordered from any of a large number of suppliers, e.g., ThermoFisher, Biolytic, IDT, Sigma-Aldritch, GeneScript, etc. In some embodiments, a gRNA expression vector backbone is used (e.g., from Addgene). In some embodiments, a guide RNA target sequence (e.g., a 19-bp target sequence) is integrated into an oligonucleotide comprising homology with the gRNA expression vector, and after PCR purification is inserted into the linearized gRNA expression vector. In some embodiments, the guide RNA is produced by in vitro transcription, e.g., using the MEGAscript

T7 High Yield Transcription Kit (Ambion). In some embodiments, guide RNAs (e.g., as synthesized or produced in vitro), are introduced into cells, e.g., as RNPs together with the fusion proteins. In some embodiments, vectors encoding the guide RNAs are introduced into cells (e.g., the cells in which a genomic modification may be detected), such that the guide RNAs are expressed in the cells.

### 5. Introduction into cells

Various methods can be used to introduce the fusion proteins and/or guide RNAs into cells (i.e., cells in which a potential mutation or editing event is detected using the present methods). In some embodiments, the fusion proteins and/or guide RNAs are introduced by introducing one or more polynucleotides encoding the fusion proteins or guide RNAs into the cells, such that the fusion protein or guide RNA are expressed in the cells. The polynucleotides can be introduced, e.g., using a viral vector, or by transfecting naked DNA or RNA. In some embodiments, the polynucleotides comprise an expression cassette comprising a coding sequence encoding a fusion protein or guide RNA, operably linked to a promoter.

Any of the well-known procedures for introducing foreign nucleotide sequences into cells may be used (e.g., to introduce vectors encoding the fusion proteins and/or guide RNAs into cells for subsequent binding to target sequences and detection of luminescence, or to introduce into host cells for expression of fusion proteins). These include the use of calcium phosphate transfection, polybrene, protoplast fusion, electroporation, liposomes, microinjection, plasma vectors, viral vectors and any of the other well-known methods for introducing cloned genomic DNA, cDNA, synthetic DNA, or other foreign genetic material into a host cell (see, e.g., Sambrook and Russell, supra). It is only necessary that the particular genetic engineering procedure used be capable of successfully introducing at least one gene into the host cell capable of expressing the recombinant polypeptide. In some embodiments, fusion protein constructs are generated using, e.g., the Gibson Assembly method (New England Biolabs). In some embodiments, a vector such as a pCDNA3-dCas9 vector is used. In some embodiments, the vector is used to transform bacterial cells, e.g., competent E. coli cells, and clones positive for the desired NanBiT insert are identified. In some embodiments, the fusion proteins comprise a tag such as an HA or Flag tag.

After the expression vector is introduced into appropriate host cells, the transfected cells are cultured under conditions favoring expression of the fusion protein or guide RNA. The cells can be screened for the expression of the protein or guide RNA. General methods for screening gene expression are well known among those skilled in the art. First, gene expression can be detected at the nucleic acid level. A variety of methods of specific DNA and RNA measurement using nucleic acid hybridization techniques are commonly used (e.g., Sambrook and Russell, supra). Some methods involve an electrophoretic separation (e.g., Southern blot for detecting DNA and northern blot for detecting RNA), but detection of DNA or RNA can be carried out without electrophoresis as well (such as by dot blot). The presence of nucleic acid encoding a fusion protein in transfected cells can also be detected by PCR or RT-PCR using sequence-specific primers.

Second, gene expression, e.g., of fusion proteins, can be detected at the polypeptide level. Various immunological assays are routinely used by those skilled in the art to measure the level of a gene product, particularly using polyclonal or monoclonal antibodies that react specifically with a fusion prtotein (e.g., Harlow and Lane, Antibodies, A Laboratory Manual, Chapter 14, Cold Spring Harbor, 1988; Kohler and Milstein, Nature, 256: 495-497 (1975)). Such techniques require antibody preparation by selecting antibodies with high specificity against the peptide. The methods of raising polyclonal and monoclonal antibodies are well established and their descriptions can be found in the literature, see, e.g., Harlow and Lane, supra; Kohler and Milstein, Eur. J Immunol., 6: 511-519 (1976).

In some embodiments, the first guide RNA and the first fusion protein, and the second guide RNA and the second fusion protein, are first produced in vitro and assembled into ribonucleoproteins (RNPs), and the RNPs are then introduced into the cell, e.g., by lipofection.

Any cell type, including animal cells, mammalian cells, or human cells, can be used in the present methods. Also included are cells of other primates; mammals, including commercially relevant mammals, such as cattle, pigs, horses, sheep, cats, dogs, mice, rats; birds, including commercially relevant birds such as poultry, chickens, ducks, geese, and/or turkeys.

In some embodiments, the two fusion proteins are introduced into the cell in different relative amounts, e.g., vectors encoding the two proteins are transfected into cells at different relative levels, e.g. a ratio of from 1:50 to 50:1, or RNPs comprising the two proteins are introduced at different levels. In some embodiments, the molar quantity of one of the fusion proteins, e.g., the fusion protein comprising LgBiT-dCas9, is lower than that of the other fusion protein, e.g., is 5%, 10%, 15%, or 20% of the molar quantity of the other fusion protein. In particular embodiments, the fusion protein comprising SmBiT is introduced at a molar excess of about 10:1 relative to the fusion protein comprising LgBiT.

The guide RNA can be introduced into the cell at any of a variety of levels relative to the fusion proteins. In some embodiments, the ratio of guide RNA (or a polynucleotide encoding a guide RNA) is introduced into the cells at a ratio of, e.g., about 1:1, 5:1, 10:1, 15:1, 20:1 or more of guide RNA:total fusion protein (e.g. NanoBiT) plasmid. In some embodiments, e.g., when fusion proteins and guide RNAs are introduced into cells as RNPs, the ratio of fusion protein to guide RNA is, e.g., about 1.5:1, 1.4:1, 1.3:1, 1.2:1, 1.1:1, 1:1, 1:1.1, 1:1.2, 1:1.3, 1:1.4, or 1:1.5.

### 6. Detecting luminescence

The efficacy of the present methods, e.g., with respect to different fusion proteins, different target sequences, different target sequence arrangement and spacing, the use of plasmid-based or RNP-based methods of introducing fusion proteins and guide RNAs, different ratios of reporter fragments and/or guide RNAs, different cell types, etc., can be assessed in any of a number of ways. In some embodiments, the components of the system (e.g., fusion proteins and guide RNA, and optionally a target DNA sequence) are introduced into cells, e.g., HEK293T, HeLa, MCF7, HCT116, K563, JLat, or other cells, a substrate (such as furimazine) is added, and the signal detected both in the presence and absence of the target DNA (or one or more of the other components such as the guide RNA). For example, in some embodiments, a luminometer is used to measure luminescence across whole cell populations. In some embodiments, a SpectraMax M5 Microplate Reader (Molecular Devices) is used. In some embodiments, a kit such as the Nano-Glo Live Cell Assay System (Promega) is used. In some embodiments, a fluorescence microscope is used to measure luminescence in single cells. In some embodiments, a system such as the PerkinElmer IVIS Spectrum Bioluminescence Imaging System is used, e.g., to image many cells in a culture simultaneously. In some embodiments of any of the herein-described methods, the system (e.g., fusion proteins and guide RNA) produces an increase in luminescence (e.g., RFU/RLU) of at least about 10%, 20%, 30%, 40%, 50%, 60%, 70%, 80%, 90%, 100%, 200%, 300%, 400%, 500%, 1000%, 1500%, 2000%, or more, or of at least about 1, 2, 3, 4, 5, 6, 7, 8, 9, 10, 15, 20, 25, or more fold, e.g., in the presence of the target DNA vs. in the absence of the target DNA, or in the presence of the fusion proteins and the guide RNA vs. in the presence of the fusion proteins alone (i.e., without one or both guide RNAs). In some embodiments, changes in luminescence can be evaluated using receivor operating characteristic (ROC) analysis. In some embodiments, the area-under-the-curve (AUC) detected using the present methods is at least about 0.8, 0.85, 0.9, 0.91, 0.92, 0.93, 0.94, 0.95, or greater.

### 7. Compositions and Kits

The present disclosure also provides compositions, e.g., any of the herein-described fusion proteins, guide RNAs, or polynucleotides encoding any of the herein-described fusion proteins or guide RNAs, as well as expression cassettes or vectors comprising any of the herein-described polynucleotides, and host cells comprising any of the herein-described fusion proteins, guide RNAs, expression cassettes, vectors or polynucleotides.

The present disclosure also contemplates kits comprising compositions or components of the present disclosure, e.g., fusion proteins, guide RNAs, RNPs, substrates (e.g., furimazine), cells, polynucleotides or vectors encoding fusion proteins and/or guide RNAs, as well as, optionally, reagents for, e.g., the introduction of the components into cells. The kits can also comprise one or more containers or vials, as well as instructions for using the compositions in order to detect specific DNA sequences (e.g., modified genomic or plasmid sequences) in cells according to the methods described herein.

### 8. EXAMPLES

The present invention will be described in greater detail by way of specific examples. The following examples are offered for illustrative purposes only, and are not intended to limit the invention in any manner. Those of skill in the art will readily recognize a variety of noncritical parameters which can be changed or modified to yield essentially the same results.

### Example 1. A Dimeric, Luminescent Biosensor for Imaging Unique DNA Sequences in Individual Cells

An extensive arsenal of biosensing tools has been developed based on the clustered regularly interspaced short palindromic repeat (CRISPR) platform, including those that detect the presence of specific DNA sequences both in vitro and in live cells. To date, DNA biosensing approaches have traditionally used monomeric fluorescent reporter-based fusion probes. Such “always-on” probes typically do not adequately differentiate between unbound and bound forms of the probe and often require tandem arrays to increase signal-to-noise, among other issues. Herein we describe a luminescence-based, dimeric DNA sequence biosensor that provides a sensitive readout for DNA sequences through proximity-mediated reassembly of two independently optimized fragments of NanoLuc luciferase (NLuc), a small, bright reporter. Reconstitution of NLuc becomes more favorable upon binding of two guide RNAs (gRNAs) to two DNA target sites with a defined orientation and spacing. Using this “turn-on” probe, we demonstrate rapid and sensitive detection of as low as 190 amol transfected target DNA and single-copy genomic loci in live cells, presenting a reliable and widely applicable approach for DNA biosensing.

### Introduction

A promising alternative to these and other destructive DNA detection assays could be the direct biosensing of edited DNA sequences in living cells. In recent years, the CRISPR/Cas gene editing system has been modified for imaging endogenous genomic loci, but the vast majority of current approaches utilize monomeric fluorescent reporter-based biosensors, such as dCas9-GFP (15-22). (FRET) (23-34). However, each monomeric sensor molecule produces a signal whether bound to its target DNA or not, resulting in a high fluorescent background that negatively impacts the signal-to-noise ratio. For this reason, such “always-on” sensors must rely on obtaining a high local concentration of probes to distinguish signal from noise, limiting their use to highly repetitive elements that can be targeted by one gRNA or to unique sequences targeted by 50 or more gRNAs.

In contrast, dimeric “turn-on” DNA biosensors offer the possibility of achieving signal production solely upon binding of two subunits to the target DNA and reassembly of a bright reporter. Luminescent reporters offer an attractive alternative to fluorescent reporters in biosensing experiments for several reasons. In particular, cellular background signal is essentially nonexistent during luminescence experiments due to the necessity of light production from a catalytic reaction of an enzyme with its substrate (33). Thus, luminescence-based assays can facilitate highly sensitive measurements of luminescent reporter activity. In terms of expected signal-to-noise ratios, luminescence-based biosensing approaches would be expected to be much more sensitive to the presence of the underlying physicochemical target than fluorescence-based biosensing approaches.

One advantage of the extensive collection of currently available fluorescent reporters is that they remain brighter than currently available luminescent reporters (35). However, a relatively new luciferase, NanoLuc (NLuc) bridges this gap in signal intensity. NLuc offers several advantages over direct competitors such as Firefly (FLuc) and Renilla (RLuc) luciferases including enhanced stability, significantly smaller size, and >150-fold enhancement in luminescence output (36-37). Furthermore, the substrate for NLuc, furimazine, is more stable and exhibits decreased levels of background activity (36-37). Taking these points into consideration, we developed a dimeric DNA sequence biosensor based on the NanoLuc Binary Technology (NanoBiT) complementation reporter system recently created for NLuc (38) and catalytically inactive Cas9 (dCas9) from Streptococcus pyogenes. Due to the high dissociation constant (Kd=190 μM) and extremely low catalytic activity of the NanoBiT complementation reporter system subunits—termed LgBiT and SmBiT—they must be brought into close proximity in order to reassemble full-length NLuc. Thus, we designed an RNA-guided approach that increases favorability of NanoBiT association upon binding of two single guide RNA (gRNA)-driven ribonucleoprotein complexes (RNPs) to two target sites with a specific orientation and spacing on the DNA. Across several cell-based delivery approaches, we achieved approximately 2.5-20-fold increase in signal in live populations of cells transfected with the dimeric biosensor and various target DNA scaffolds compared to populations transfected with the dimeric biosensor but no target DNA. Subsequently, we tested the sensitivity of the biosensor on specific endogenous genomic DNA sequences across multiple cell lines and compared the signal-to-noise of this approach to a common fluorescence-based method. Finally, we conducted CRISPR-Cas9 editing experiments on several genomic loci and were able to detect these edits by signal-to-noise differences between homozygous mutant and wild type cells.

### Results

### Strategy for Designing a Dimeric, RNA-Guided DNA Biosensor

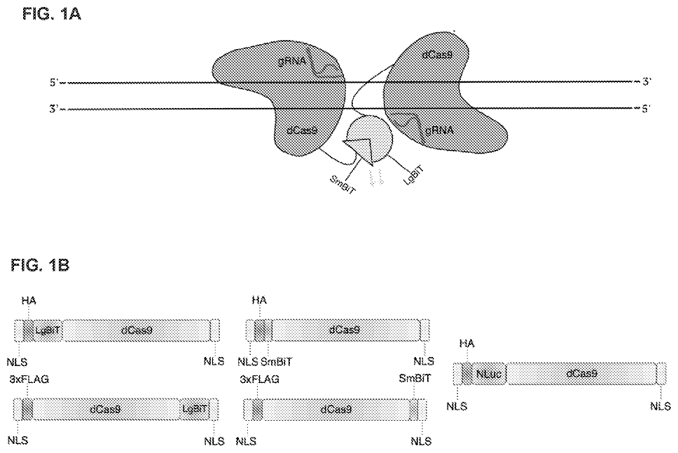

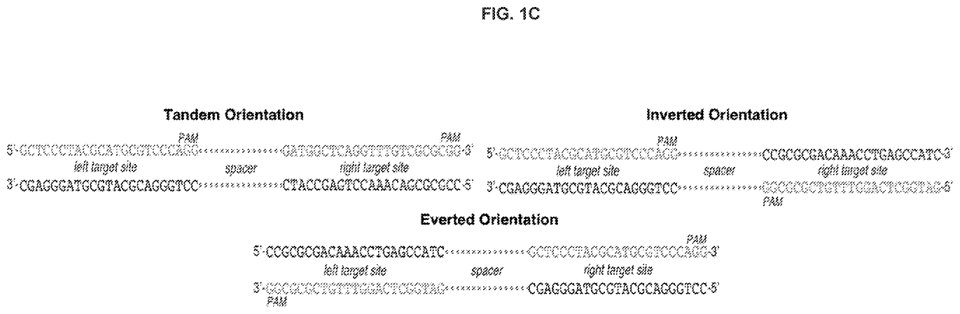

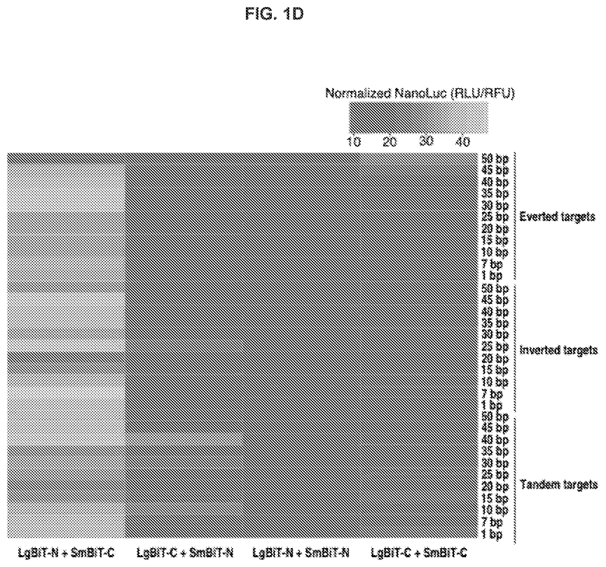

To design a live cell DNA sequence biosensor, we fused two independently optimized protein fragments of NLuc, LgBiT and SmBiT, to a catalytically inactive Cas9 from S. pyogenes (dCas9). We envisioned a system of high fidelity and specificity where a bright luminescent signal would be produced upon binding of two guide RNAs (gRNAs) to two target sites with a specific orientation and spacing between them (FIG. 1A). Primarily considering signal-to-noise maximization, we sought to choose a specific NLuc truncation point that minimized nonspecific auto-association of the protein fragments in the nucleus. Since the dissociation constant (Kd) of LgBiT and SmBiT is 190 μM, we predicted that this specific protein complementation system should exhibit very low levels of background nuclear association and thus was particularly well suited for this purpose. Furthermore, due to the requirement of two unique gRNAs in a split probe system, we predicted signal production from off-target DNA binding events by dCas9 would be extremely unlikely.

### Construction and Optimization of a Dimeric, RNA-Guided DNA Sequence Biosensor

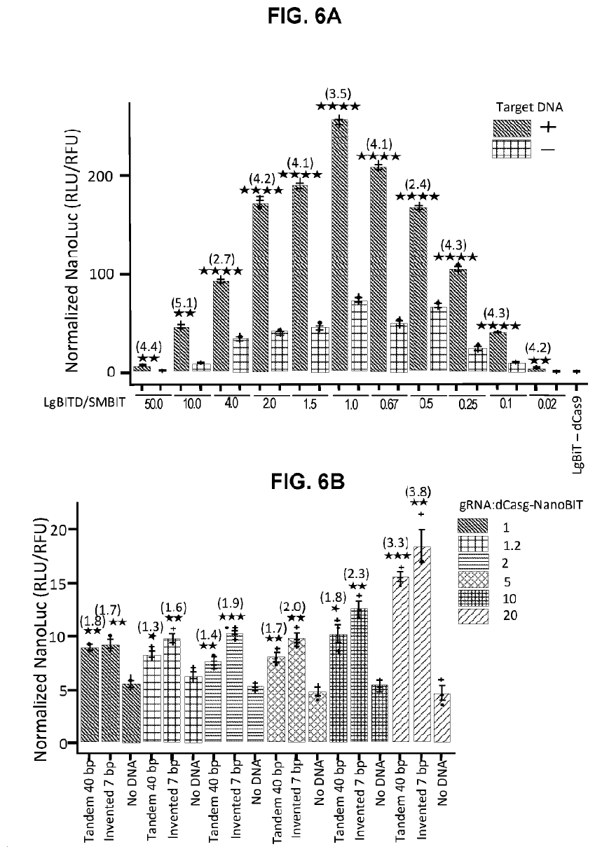

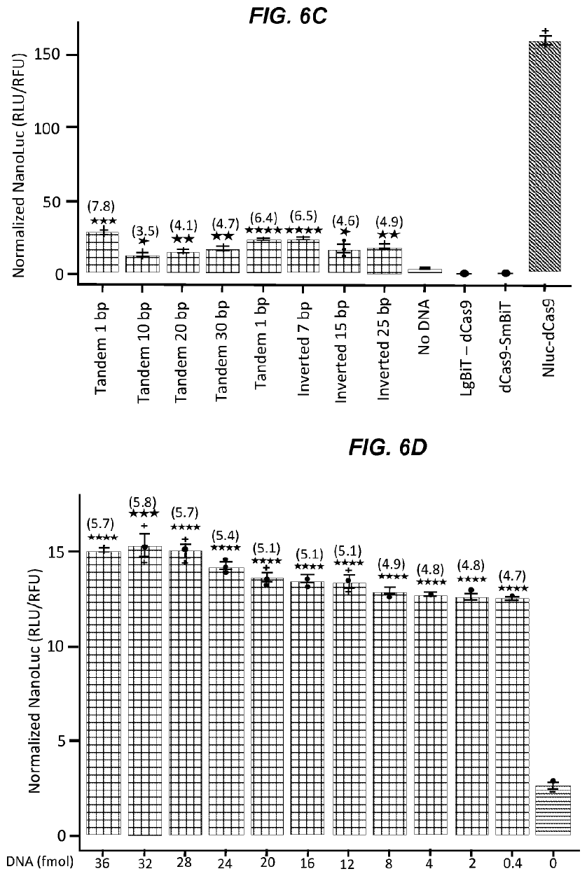

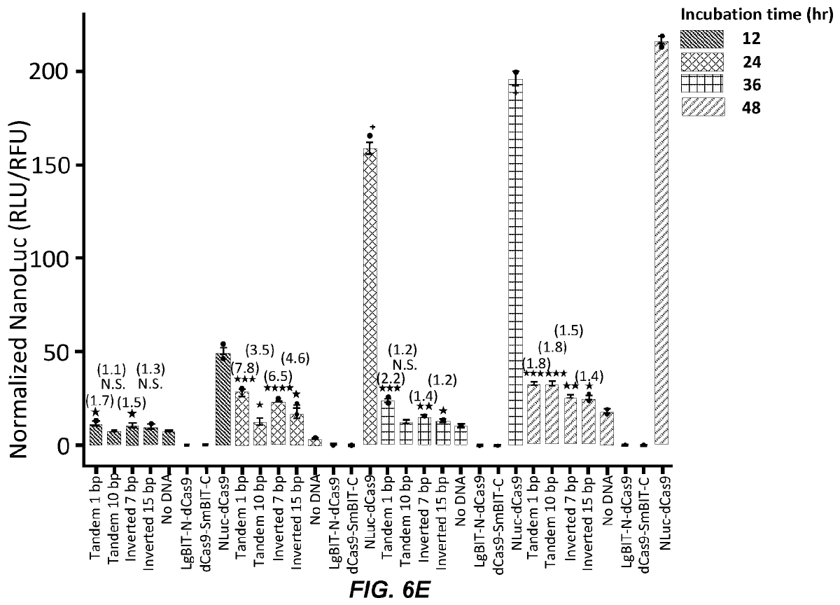

We initially constructed five fusion proteins: two in which the LgBiT and SmBiT were fused to the carboxy-terminus of dCas9 (dCas9-LgBiT and dCas9-SmBiT), two in which they were fused to its amino-terminus (LgBiT-dCas9 and SmBiT-dCas9) and one in which full-length NLuc was fused to the amino-terminus of dCas9 (NLuc-dCas9) (FIG. 1B). Subsequently, we produced 33 plasmids each harboring one copy of a DNA target site scaffold containing two SpCas9 gRNA target sites in three orientations with 1, 7, 10, 15, 20, 25, 30, 35, 40, 45, and 50 base pair (bp) spacer sequences between them. We defined the three possible orientations of target sites by configuration and phase on the double helix, including tandem, inverted, and everted target sites (FIG. 1C). We next sought to define the optimal molar transfection ratios of LgBiT:SmBiT and NanoBiT:gRNA, limit of detection for target DNA, and ideal incubation time using transient transfection of DNA in HEK 293T cells. For simplicity, initial experiments used only LgBiT-dCas9 and dCas9-SmBiT fusion proteins on tandemly orientated target sites with 10-bp spacers, as this design was expected to bring the luciferase subunits into close proximity based on initial modeling in PyMOL. To determine the optimal ratio of LgBiT:SmBiT, plasmids expressing LgBiT-dCas9 and dCas9-SmBiT fusion proteins were co-transfected in ratios ranging between 50:1 and 1:50 with or without target DNA plasmids. In transfections where the amount of one dCas9-NanoBiT interaction partner was decreased, an equal amount of inert pUC19 DNA was included in the transfection mix. Signal-to-noise was maximal at approximately 5-fold using a 10:1 ratio of LgBiT-dCas9 to dCas9-SmBiT in live HEK 293T cells (FIG. 6).

Signal-to-noise may depend on the relative concentrations of the biosensor components in the nucleus, including dCas9-NanoBiT fusion proteins, the gRNAs, and target site DNA. To optimize these parameters, we first varied the dCas9-NanoBiT:gRNA plasmid ratio. These parameter optimizations were performed with controls to establish the background level of dCas9-NanoBiT auto-association where DNA target plasmids were not transfected, controls to ensure association of NanoBiT fusion proteins was occurring where LgBiT-dCas9 and dCas9-SmBiT were each transfected separately, and controls to establish an upper bound for the theoretically achievable signal due to NLuc reassembly where an equimolar amount of NLuc-dCas9 plasmid to the total molar amount of dCas9-NanoBiT plasmid used in other conditions was transfected. We observed a modest increase in the signal-to-noise to approximately 8-fold by using a 20:1 ratio of gRNA:total NanoBiT plasmid (FIG. 6). Using this 20:1 ratio for the gRNA plasmid, we then varied the molar amount of target plasmid in transfection between 0.4 and 36 fmol. We found very little dependence of signal-to-noise on the molar amount of target DNA transfected (FIG. 6), and therefore used the lowest amount of target DNA, 0.4 fmol, for all subsequent experiments. However, there appeared to be an ideal incubation time of 24 hours between transfection and measurement of signals at which signal-to-noise peaked (FIG. 6). Having established these optimal parameters for the assays, we investigated the differences in luminescent signal output between four possible protein configurations of dCas9-NanoBiT fusion constructs binding to the three possible target site orientations (tandem, inverted, and everted) with 11 different spacings (33 target DNA combinations total). Hypothesizing that fusion protein orientation and target DNA orientation might interact to create a synergistic effect on signal output, we conducted a two-way ANOVA assuming there was an interaction between these two variables. Significant variation in the efficiency of NLuc reassembly was observed across conditions (FIG. 1D), with fusion protein orientation and target DNA orientation being associated with significant differences in luminescent signal output (p<0.0001 and p<0.05, respectively, two-way ANOVA, see Table 1). The relationship between signal output and fusion protein orientation was also shown to depend on target DNA orientation and vice versa (F(96, 264)=2.064, p<0.0001, two-way ANOVA, see Table 1) indicating that these results are affected by an interaction between fusion protein and target DNA orientations. We then used Tukey's Honestly Significant Difference post-hoc test to determine which group means were significantly different from each other. This analysis showed that signal sets from all pairs of fusion protein orientations differed significantly from one another except between the dCas9-LgBiT+SmBiT-dCas9 and dCas9LgBiT+dCas9-SmBiT pairs (p<0.0001, Tukey HSD, see Table 2). The LgBiT-dCas9+dCas9-SmBiT protein configuration clearly produced a significantly higher set of luminescent signals, (p<0.0001 for three pairwise comparisons, Tukey HSD, see Table 2). Furthermore, the conditions that produced significantly higher signals (and highest signal-to-noise as background auto-association signals were similar across all four fusion protein orientations) were tandem 40-bp (98/131 pairwise comparisons with p<0.05, Tukey HSD) and inverted 7-bp (99/131 pairwise comparisons with p<0.05, Tukey HSD) DNA target plasmids paired with LgBiT-dCas9 and dCas9-SmBiT fusion proteins. Across the data set, only one pairwise comparison between DNA target orientations differed significantly (p<0.05 for tandem 40-bp compared to inverted 20-bp, Tukey HSD). However, many target DNA orientations paired with LgBiT-dCas9 and dCas9-SmBiT fusion proteins exhibited significantly higher signal output and signal-to-noise (>30/131 pairwise comparisons with p<0.05, Tukey HSD), including the tandem 1-bp, tandem 10-bp, tandem 45-bp, inverted 25-bp, inverted 45-bp, everted 30-bp, and everted 35-bp DNA target configurations. Interestingly, the everted 50-bp DNA target plasmid paired with dCas9-LgBiT and dCas9-SmBiT fusion proteins also produced significantly higher signals (97/131 pairwise comparisons with p<0.05, Tukey HSD). Aiming to use fusion protein and DNA target configurations that resulted in better assembly of NLuc in transfection, we chose to deliver tandem 40-bp and inverted 7-bp DNA target plasmids with LgBiT-dCas9 and dCas9-SmBiT fusion proteins in future experiments.

### Testing an RNP-Based DNA Biosensor Delivery Approach in Live Cells

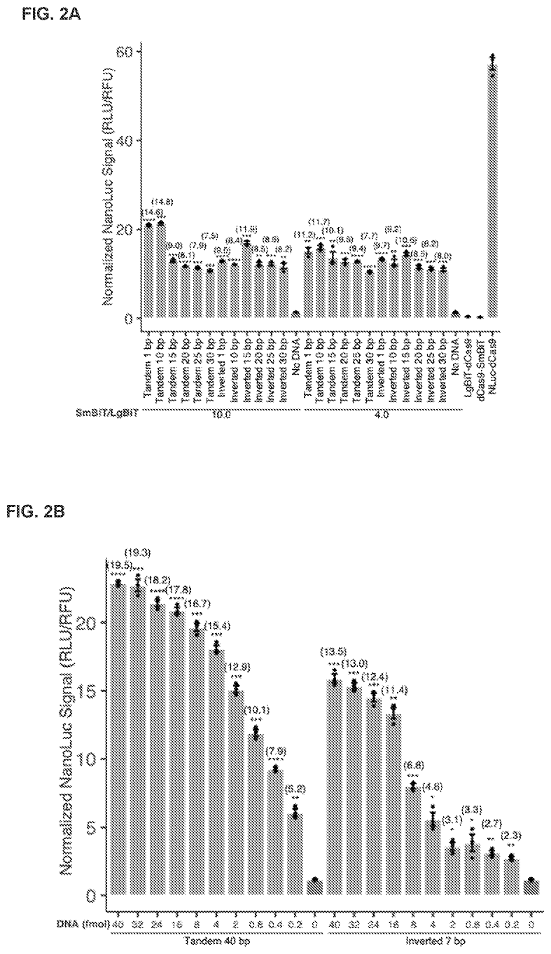

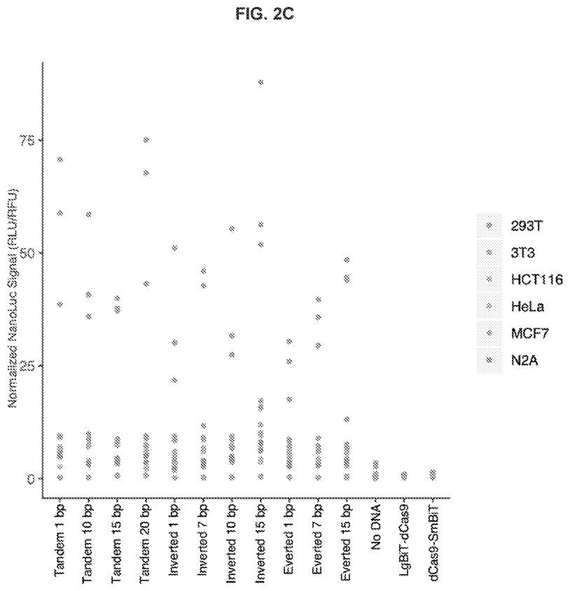

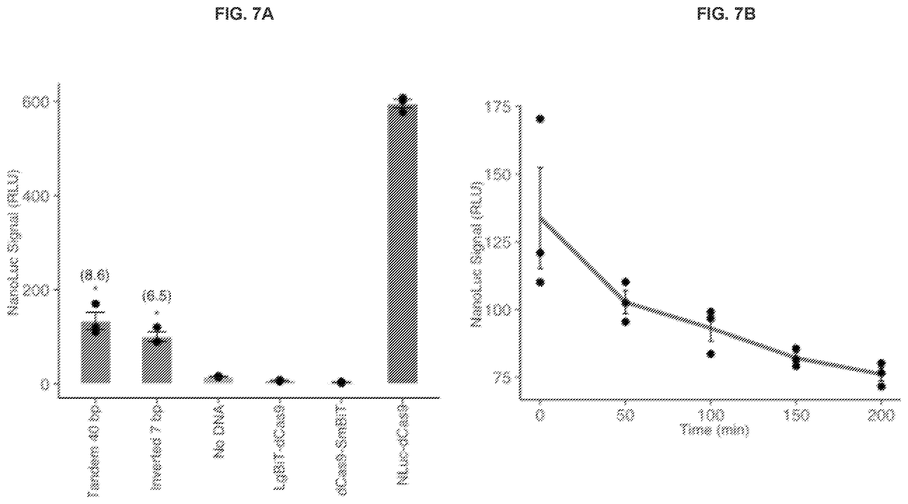

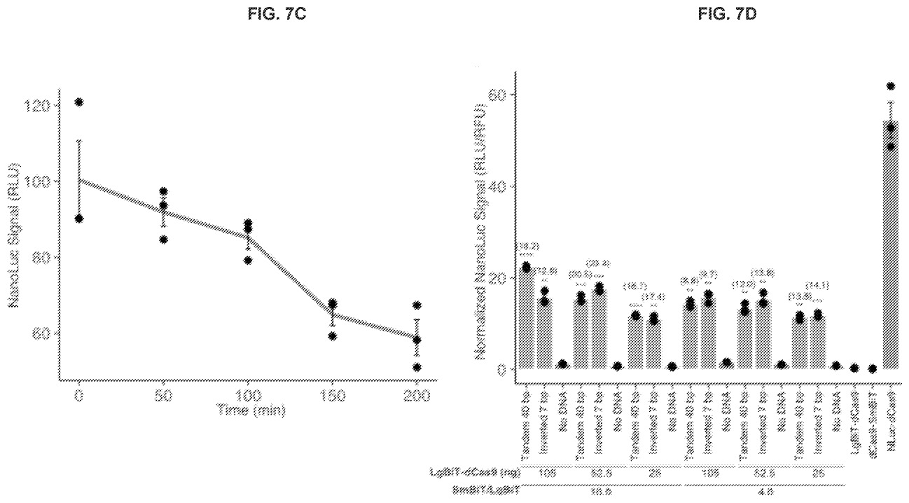

Due to relatively high background signal in the negative control cell populations with no target DNA transfected, we theorized that delivery of the dCas9-NanoBiT fusion proteins and gRNAs as ribonucleoprotein complexes (RNPs) would provide better control of initial nuclear protein concentration and allow it to decrease steadily after administration in contrast to the large increase and slow decrease associated with plasmid-based expression. The steadily decreasing RNPs might therefore provide a strong target signal while reducing the background signal, resulting in more sensitive detection of the DNA target sequence of interest. Thus, we expressed and purified fusion proteins from HEK 293T using immunoprecipitation, complexed them with in vitro-transcribed gRNAs, and validated NanoLuc signal output from the resulting dCas9-NanoBiT RNPs and from the NLuc-dCas9 RNP. Notably, relative signal differences in vitro between the dCas9-NanoBiT RNPs binding target DNA and NLuc-dCas9, the LgBiT alone, and the SmBiT alone controls remained largely identical with the exception of the background signal from auto-association of LgBiT and SmBiT, which was markedly lower relative to all other signals compared to previous plasmid-based delivery experiments (FIG. 7). Signal output decayed in vitro when 560 fmol total LgBiT-dCas9 and dCas9-SmBiT RNPs were mixed with 40 fmol tandem 40-bp and inverted 7-bp target DNA plasmids to the point where 59% and 57% of the original signal was present 200 minutes after complexation, respectively (FIG. 7). In complexing the RNPs, we used a 1:1.2 ratio of purified fusion protein:gRNA. In accordance with our characterization of the plasmid-based system, we initially chose to test tandem 40-bp and inverted 7-bp DNA target plasmids along with the LgBiT-dCas9 and dCas9-SmBiT fusion proteins in live cell transfections. Subsequently, we delivered 560, 280, and 130 fmol total of the RNPs with dCas9-SmBiT and LgBiT-dCas9 fusion proteins in 10:1 and 4:1 molar transfection ratios to HEK 293T cells along with 40 fmol target DNA plasmids with tandem target sites 40 bp apart and inverted target sites 7 bp apart using Lipofectamine CRISPRMAX (FIG. 7). The range of signal-to-noise ratios obtained using this approach was approximately 13-fold to 18-fold, a substantial improvement over plasmid-based delivery. We then tested the RNP-based delivery method using 560 fmol total RNPs on 12 additional DNA target sequence scaffolds (40 fmol each), and the range of signal-to-noise ratios was approximately 7.5-fold to 20-fold, underscoring the efficiency of this delivery approach (FIG. 2A). As we were delivering many copies of the target sequences in transfections, we sought to test the limit of detection for RNP-based delivery of the biosensor. We found that there was a sharper negative response in signal-to-noise when target DNA concentration was decreased in RNP transfection compared to plasmid transfection of biosensor components (FIG. 2B). At the minimum amount of target site DNA transfected of 0.2 fmol, signal-to-noise was approximately 6-fold for LgBiT-dCas9 and dCas9-SmBiT RNPs binding the tandem 40-bp target DNA plasmid and 3-fold for LgBiT-dCas9 and dCas9-SmBiT RNPs binding the inverted 7-bp target DNA plasmid. We then tested the same RNP biosensor delivery conditions across five other cell lines, with similar signal-to-noise ranges but much lower absolute signals compared to HEK 293T (FIG. 2C).

### Live Single-Cell Biosensor Imaging Using a Standard Light Microscope or IVIS System

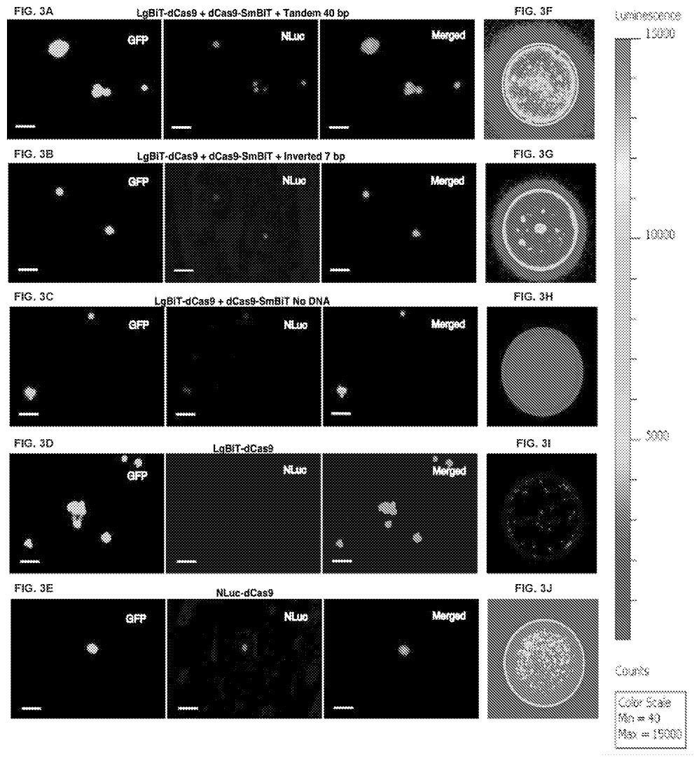

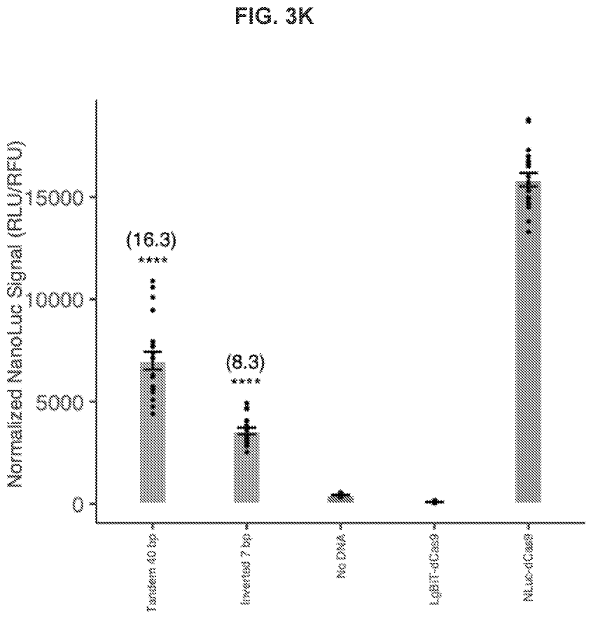

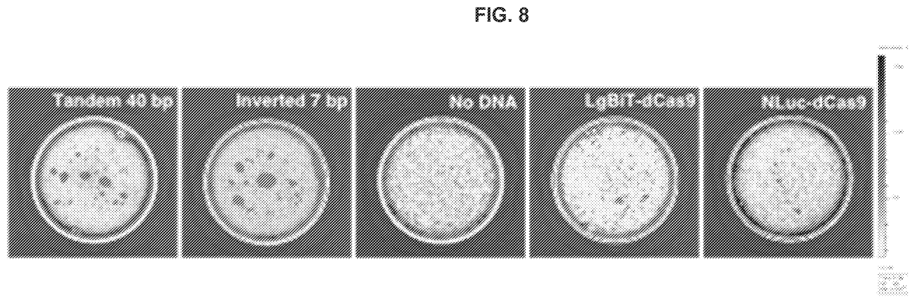

After obtaining the best set of plasmid and RNP-based delivery conditions for our DNA sequence biosensor in live cells, we sought to confirm the signal-to-noise ratios obtained through orthogonal approaches. In addition to our approach using a luminometer to measure luminescence across whole well cell populations, we envisioned a platform for measurement of luminescence from our biosensor in single cells on relatively common imaging equipment. To this end, we modified an upright fluorescence microscope for imaging the relatively low light intensities associated with NLuc and other luminescent reporters. For example, cells were placed in a dark box with all light sources covered or off, and exposure times were lengthened (see Methods). 560 fmol total purified dCas9-SmBiT and LgBiT-dCas9 biosensor proteins were co-transfected in HEK 293T cells along with 40 fmol DNA target plasmids containing either tandem 40 bp or inverted 7 bp target sites and 0.2 fmol pMAX-GFP plasmid as a normalization control (FIGS. 3A-3B, respectively). Intensity of signals from these images were compared to those from an auto-association background control without target DNA (FIG. 3C), a LgBiT-dCas9 fusion construct expressed alone (FIG. 3D) and a full-length NLuc-dCas9 positive control construct (FIG. 3E). As an alternative approach, we also measured the same set of NLuc luminescent signals on the PerkinElmer IVIS Spectrum Bioluminescence Imaging System (FIGS. 3F-3J). GFP signal images for normalization were obtained for all conditions (FIG. 8). Although the required equipment may not be as accessible as a light microscope, the IVIS system has the advantage of imaging many cells in a culture dish simultaneously, allowing many imaging experiments to performed with minimal time and effort. For these images, we drew and integrated circular regions of interest (ROIs) around regions containing cell nuclei within the LivingImage software associated with the IVIS Spectrum, obtaining a comparable range of signal-to-noise (FIG. 3K).

### Live Single-Cell Biosensor Imaging of Repetitive and Unique Endogenous Genomic Sequences

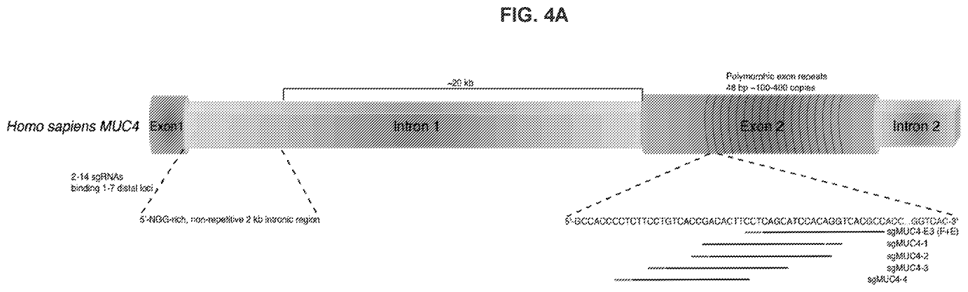

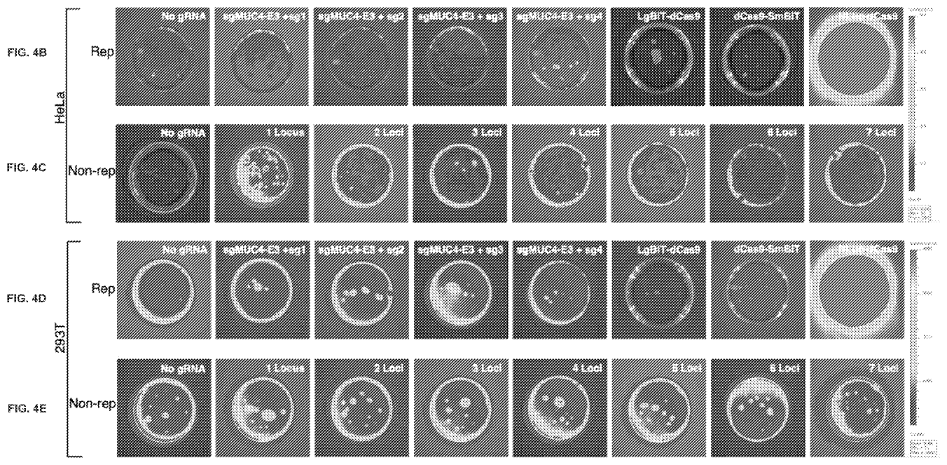

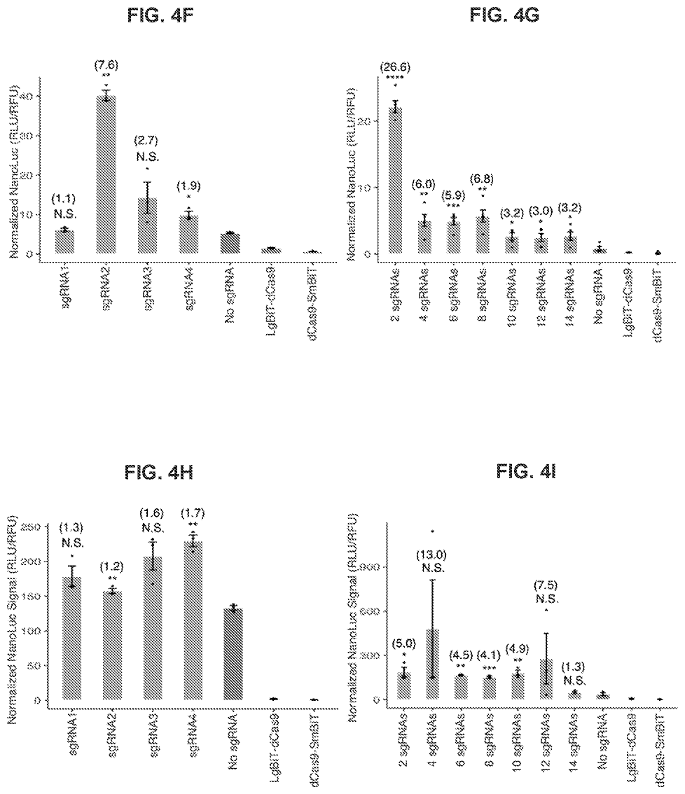

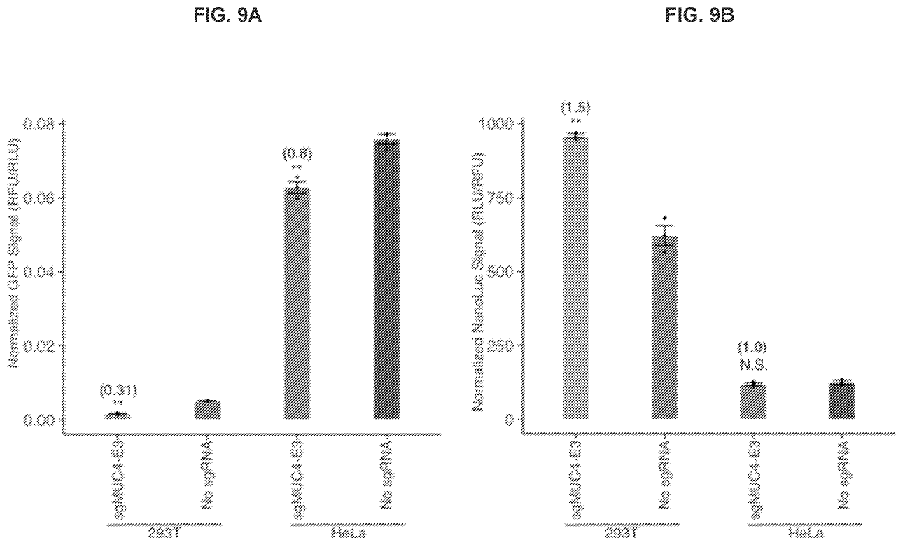

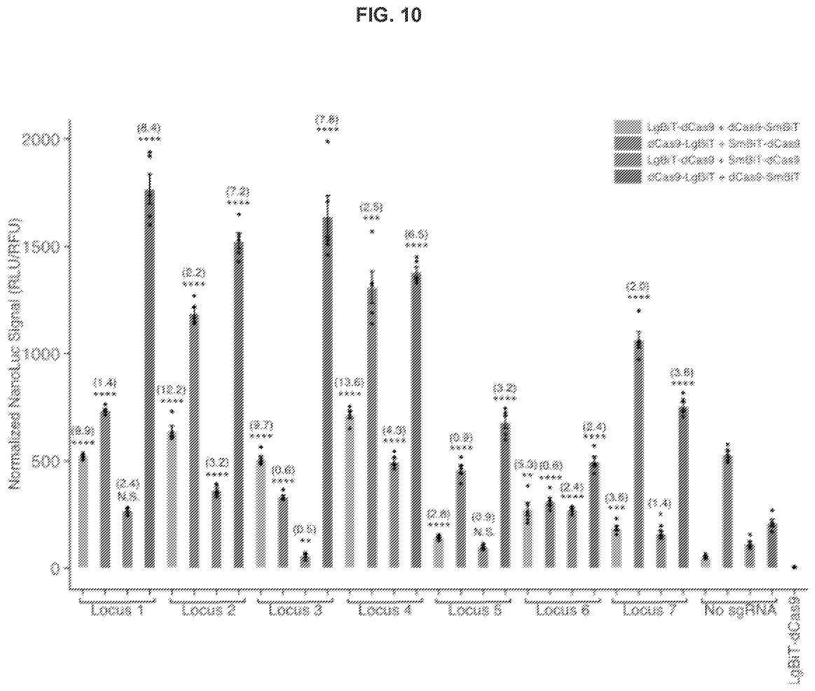

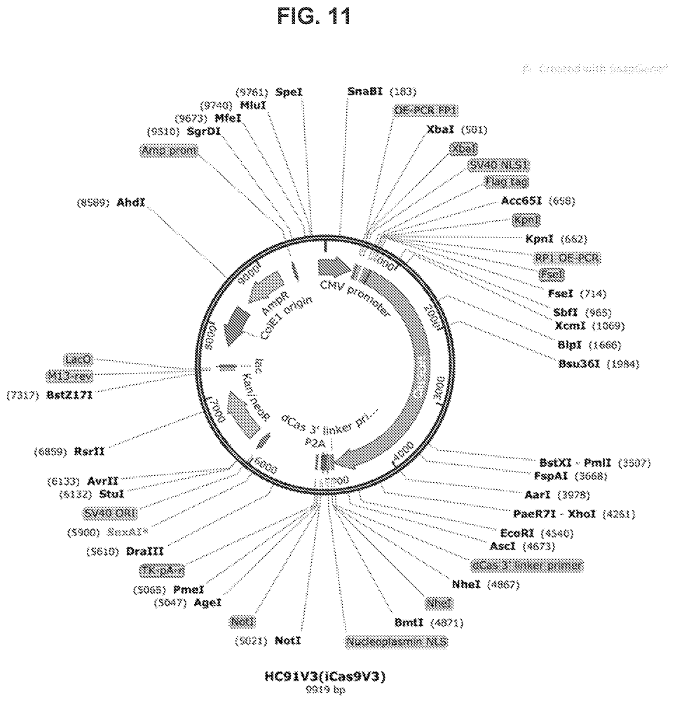

To determine the applicability of our dimeric luminescent biosensor to imaging endogenous copy number DNA sequences, we first compared its sensitivity to that of both a previously described dCas9-EGFP monomeric fluorescent probe (15) and the monomeric NLuc-dCas9 probe from our study. We used a single optimized gRNA, sgMUC4-E3(F+E) (15) to direct these probes to bind a region of polymorphic 48-bp repeats of copy number between approximately 100 and 400 within exon 2 of the human MUC4 locus (FIG. 4A). Using integration of nuclear signals obtained on the IVIS Spectrum, we found that both monomeric probes had comparable signals when binding the tandem repeats compared to a background condition with no RNA-guided DNA binding across two cell lines (FIG. 9). We then used sgMUC4-E3(F+E) as an anchor gRNA to direct our dimeric luminescent probe to bind the same repetitive region of MUC4 and constructed four gRNAs with unique spacer lengths and orientations around it (see Example 2, Supplementary Methods 5 for target sequences and construction methods). We observed differences in biosensor sensitivity that varied based on cell line and target site configuration (FIGS. 4B, 4D). For example, signal-to-noise peaked at approximately 7.5-fold in HeLa cells (FIG. 4F) and approximately 2-fold in HEK 293T cells (FIG. 4H). It should be noted that these peak signal-to-noise ratios were obtained with different gRNA pairings in each cell line. However, since the majority of loci within the human genome are non-repetitive, a utility of more profound value would be the potential of our dimeric luminescent biosensor to detect such low copy number sequences. To this end, we targeted the non-repetitive region of intron 1 of the human MUC4 locus with 1-7 pairs of unique gRNAs tiling along the locus with at least 200 bp between pairs to avoid interactions between biosensor components at different binding sites (FIG. 4A, Example 2, Supplementary Methods 5 for target sequences and construction methods). Using this approach, we again observed cell type-specific and target site orientation-specific differences in biosensor sensitivity but also what appeared to be dosage effects relating to number of gRNA pairs transfected (FIGS. 4C, 4E). Specifically, signal-to-noise in HeLa cells peaked at approximately 27-fold using a single pair of gRNAs at a single locus (FIG. 4G) but at approximately 13-fold in 293T cells using two pairs of gRNAs at two loci (FIG. 4I). Since the differences between signal-to-noise in the two different cell lines could be related to dosage of gRNA pairs or intrinsic chromatin structure, we conducted an experiment where each of the seven loci was bound independently and pairwise comparisons in signal-to-noise were made (FIG. 10). The signal-to-noise ratios were maximal at 52.6-fold at locus 1, 2.47-fold at locus 4, 4.33-fold at locus 4, and 8.36-fold at locus 1 for LgBiT-dCas9+dCas9-SmBiT, dCas9-LgBiT+SmBiT-dCas9, LgBiT-dCas9+SmBiT-dCas9, and dCas9-SmBiT+dCas9-LgBiT pairings of fusion proteins, respectively. Locus 1 within the MUC4 non-repetitive region has a tandem 10-bp target site DNA configuration while locus 4 has a tandem overlapping target site DNA configuration with PAM sites 4 bp apart (Example 2, Supplementary Methods 5). This confirms previous results demonstrating signal output and signal-to-noise dependence on both fusion protein orientation and target site configuration.

### Live Single-Cell Biosensor Imaging of Single-Base Changes Induced by CRISPR-Cas9 Editing

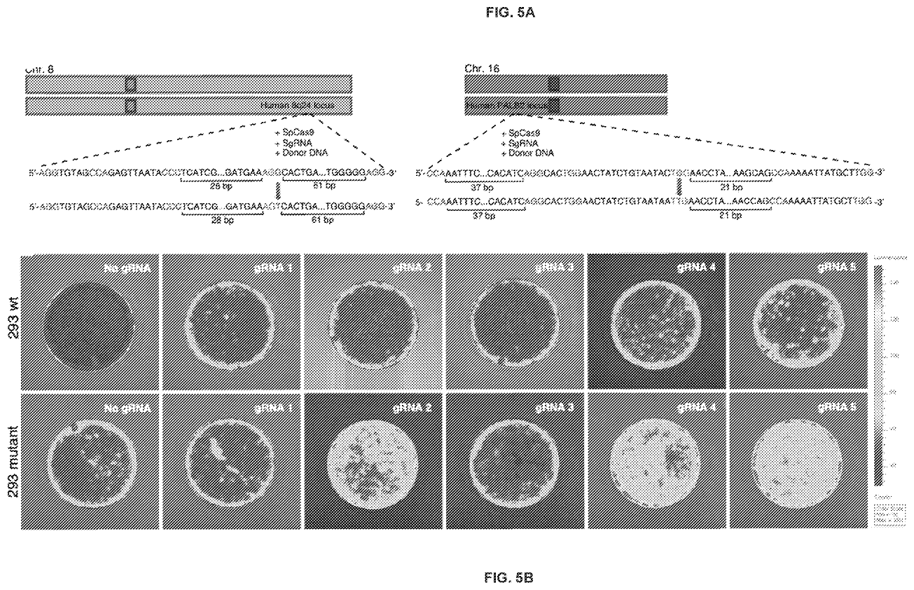

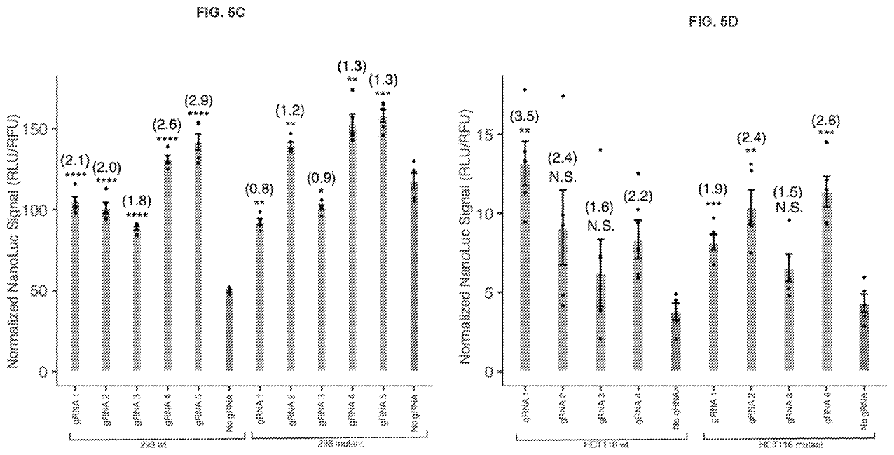

Our main goal in conceiving a dimeric luminescent biosensor was to apply it to detection of various mutations in genomic DNA sequence after targeted genome editing with CRISPR-Cas9. Thus, we created G->T missense single nucleotide polymorphisms (SNPs) at two different loci in two cell lines: within the 8q24 multi-cancer risk locus in HCT116 cells and within the PALB2 locus in 293 cells (FIG. 5A). Both SNPs were present within the PAM site of the gRNA used for editing (39) (Example 2, Supplementary Methods 6). We confirmed mutant lines were homozygous for the G->T missense mutations by isolating single edited cells by dilution plating then expanding populations and detecting specific alleles by Kompetitive Allele-Specific PCR (KASP). We hypothesized that these mutations would completely inhibit binding by the gRNA used for editing or at least make binding less efficient. Thus, we expected signal-to-noise within the mutant lines to be lower than signal-to-noise within wild-type lines. This result was most apparent when we measured signals of wild-type and homozygous mutant 293 cells receiving LgBiT-dCas9 and dCas9-SmBiT biosensor components, the gRNA used for editing, and several gRNAs of various orientations and spacer sequences around the gRNA used for editing on the IVIS spectrum. The absolute signals were higher in the mutant cell lines including the background signal where there was no gRNA-guided DNA binding, resulting in lower signal-to-noise for every gRNA pair in the mutant lines (FIG. 5B). Specifically, the signal-to-noise ratios for biosensing conditions with gRNAs 1-5 around the gRNA used for editing were 2.11-fold, 2.03-fold, 1.78-fold, 2.64-fold, and 2.85-fold in wild-type lines compared to 0.79-fold, 1.19-fold, 0.86-fold, 1.30-fold, and 1.36-fold in homozygous mutant lines (FIG. 5C). In HCT116 cells, the signal-to-noise ratios for biosensing conditions with gRNAs 1-4 around the gRNA used for editing were 3.46-fold, 2.4-fold, 1.64-fold, and 2.2-fold in wild-type cells compared to 1.89-fold, 2.4-fold, 1.51-fold, and 2.62-fold in homozygous mutant lines (FIG. 5D).

### Discussion

When we initially characterized our DNA sequence biosensor in live cells, we expected all LgBiT-SmBiT pairings, when transfected with target DNA, to show signals in a range between the NanoBiT fusion proteins expressed alone and the full-length NLuc-dCas9 fusion protein, demonstrating successful assembly of the NanoBiTs. Normalized luminescent signals for all pairings of NanoBiT-dCas9 fusion proteins in biosensing conditions was in the range of 8.94-49.3 RLU/RFU, which clearly exceeded the upper range of normalized signals for dCas9-SmBiT expressed alone (6.42-7.29 RLU/RFU) and for dCas9-LgBiT expressed alone (7.3-8.5 RLU/RFU) but was below the lower range of normalized signals for the NLuc-dCas9 fusion protein (97.52-129.08 RLU/RFU). Thus, we concluded that our dimeric DNA biosensor produced expected signal output. To emphasize the advantages of a dimeric probe over a monomeric probe, we compared signal output of two RNA-guided monomeric probes in the presence and absence of target DNA. We saw largely identical signal ranges for both dCas9-EGFP and NLuc-dCas9 monomeric probes in the presence and absence of DNA target sequences, underscoring the idea that full-length reporter-DBD fusions will result in strong signal output whether the probe is bound or unbound to target DNA in the nucleus. Thus, monomeric probes are less attractive for biosensing applications due to their inherently lower sensitivity. On the contrary, a split reporter reassembly scheme offers the possibility of strong signal output only when both subunits of the reporter come together due to a specific molecular interaction, resulting in higher sensitivity.

In initial assays, we compared our biosensing condition with target DNA to our background auto-association condition with no target DNA, which we expected to be fairly low due to the known weak binding affinity between LgBiT and SmBiT. We saw a range of normalized signals for the auto-association condition of 12.53-30.46 RLU/RFU, indicating that assembly of free floating NanoBiT fusion proteins occurred at a lower level than in the DNA biosensing condition. Furthermore, the average normalized signal across 48 auto-association wells was 15.66 RLU/ RFU, whereas the average normalized signal across all 396 biosensing condition wells was 21.45 RLU/RFU, which is a significant difference by Z-test on group means (p<0.0001, two-tailed). Taken together, these differences in signal intensities for NanoBiTs expressed in the presence of target DNA compared to NanoBiTs expressed without target DNA indicated NLuc reassembly was occurring in target cell nuclei upon RNA-guided binding of the target DNA sequence. Having successfully but relatively inefficiently detected DNA target sequences using this approach in cells, we then sought to optimize delivery conditions. In doing so, we found reducing the molar quantity of the LgBiT-dCas9 fusion protein to 10% of the original quantity in transfection increased NLuc signal output in our biosensing condition compared to our background auto-association condition in live cells. Moreover, there was a noticeable drop in signal-to-noise as the molar transfection ratio of LgBiT:SmBiT approached 1:1 in transfection. This could suggest that specific association on target DNA templates is favored and nuclear auto-association is disfavored at lower molar quantities of the LgBiT-dCas9 interaction partner. In other words, it is possible that LgBiT-SmBiT auto-association is maximized when both are available in any given molecular space at approximately 1:1 molar ratio. In addition, we found using 20-fold molar excess gRNA compared to dCas9-NanoBiT fusion proteins resulted in an increase in signal-to-noise compared to other gRNA:fusion protein ratios. This result could potentially be explained by the shorter nuclear lifetime of cellular RNAs compared to both cellular DNA and proteins (40). Since RNA molecules are degraded much quicker than their DNA and protein counterparts, transient plasmid transfection-based delivery of this biosensor may require higher initial amounts of DNA template for the gRNA to reach a steady-state level of transcription and an adequate level to form RNPs in cells. This may also explain our finding that the ideal incubation time to measure NLuc luminescence post-transfection was 24 hours. Plasmid transcription, mRNA degradation, and mRNA translation show exquisite temporal control in cells (40), and a 24-hour incubation time likely resulted in fairly stable levels of both the dCas9-NanoBiT fusion proteins and available gRNAs, allowing for high rates of gRNA-fusion protein association and DNA binding in HEK 293T cells. We predicted any parameters related to the transfection of cells, signal measurement, and imaging to be moderately cell type specific, and this was partly demonstrated by our assays testing the DNA biosensor in six different cell lines. Both the absolute signals and signal-to-noise of the biosensor varied across these lines, showing that production of fusion protein or gRNA, degradation rate of target DNA, uptake efficiency of the luminescent substrate, or attenuation of the resulting signal was variable across cell lines.

The rationale for delivering the biosensor components as RNPs was twofold. First, the delivery of the fusion proteins in plasmid form resulted in the production of all possible pairings of fusion protein and gRNA. We quickly realized that half of these RNP pairings, when bound to target DNA, would not produce a detectable signal. For example, in an experiment delivering LgBiT-dCas9 and dCas9-SmBiT fusion proteins and gRNAs 1 and 2 to cells, the gRNAs could both associate with LgBiT-dCas9 fusion proteins or both associate with dCas9-SmBiT fusion proteins. These two pairings would direct RNPs with identical NanoBiTs to bind adjacent to one another on the same target DNA vector. As a result, two LgBiT-dCas9 or two dCas9-SmBiT RNPs would transiently occupy a copy of the target DNA with no resultant NLuc reassembly or signal output. While the actual number of these unproductive assemblies from initial live cell experiments is difficult to predict, these events are not unlikely by any means. Second, as protein expression from the biosensor component plasmids was driven by the constitutive CMV promoter, control of the total concentration of free-floating nuclear RNPs was not possible. Fusion proteins may have been constitutively expressed to a very high level, making auto-association of free-floating nuclear RNPs more favorable and resulting in a measurable increase in the background signal and reduction in signal-to-noise. Third, delivery of system components in plasmid form posed a low risk of spontaneous plasmid integration into the genome. Thus, although plasmid-based delivery was a successful method for DNA biosensing, we concluded it was less desirable overall compared to RNP-based delivery. In our initial RNP-based DNA biosensing experiments, we saw a range of normalized signals for our biosensor of 0.049-0.239 RLU/RFU and average normalized signal of 0.116 RLU/RFU in the presence of target DNA compared to a range of normalized signals of 0.015-0.019 RLU/RFU and average normalized signal of 0.016 RLU/RFU in the absence of target DNA. This is a significant difference by unpaired student's t-test (p<0.0001, two-tailed). From these results, it is clear that the biosensor detects the presence of DNA in live cells more efficiently when it is delivered in the form of preassembled RNPs. We then moved away from luminometer-based measurement of luminescent signals, using two cross-sectional approaches: microscopy and bioluminescence imaging. After specifically modifying these methods for our application, we obtained similar signal-to-noise measurements for our biosensor, which further confirmed the efficacy of the RNP-based delivery approach and demonstrated amenability to multiple routes of measurement and data analysis.

We also realized that introducing DNA target sites on plasmids diluted biosensor components in transfection, provided DNA targets that were only transiently available for binding in the nucleus, and resulted in target sequence copy numbers that were likely much higher than those observed for genomic loci. Thus, we designed new gRNAs to target endogenous DNA binding sites on genomic DNA in live cells instead of introducing DNA target plasmids in transfection. We theorized that this approach would allow us to investigate the critical question of whether our biosensor would be sensitive enough to detect extremely low copy numbers. One consequence of removing DNA target site vectors from the transfection was that it necessitated a new definition of the auto-association background condition. We thus employed another auto-association condition where the biosensor was not directed to bind genomic target sites due to lack of introduced gRNA. In an analogous fashion to our preliminary assays using target DNA vectors, we first assessed whether signal output was in the expected range for our biosensor. Directing the biosensor to bind a repetitive region of the human MUC4 locus in HeLa cells, normalized luminescent signals for all pairings of NanoBiT-dCas9 fusion proteins in biosensing conditions was in the range of 5.54-42.83 RLU/RFU, which again exceeded the upper range of normalized signals for dCas9-SmBiT expressed alone (0.52-0.77 RLU/RFU) and for dCas9-LgBiT expressed alone (1.24-1.63 RLU/RFU) but was below the lower range of normalized signals for the NLuc-dCas9 fusion protein (1422.23-1951.68 RLU/RFU). Thus, we determined that our dimeric DNA biosensor produced expected signal output on endogenous copy number sequences. As before, we next compared our biosensing condition with supplied gRNA in transfection to our background auto-association condition with no supplied gRNA. We saw a range of normalized signals for the auto-association condition of 5.09-5.61 RLU/RFU, again demonstrating that assembly of free floating NanoBiT fusion proteins occurred at a lower level compared to the endogenous DNA biosensing condition. Furthermore, the average normalized signal across all 12 biosensing condition wells was 17.63 RLU/RFU whereas the average normalized signal across 3 auto-association wells was 1.46 RLU/ RFU, a disparity which is significant by unpaired student's t-test on group means (p<0.05, two-tailed). In addition, we observed differences between biosensing conditions and background conditions at the repetitive region of MUC4 in 293T cells that were significant by unpaired student's t-test on group means (p<0.0001, two-tailed). Taken together, these differences in signal intensities for RNA-guided DNA binding conditions compared to undirected conditions using the dimeric biosensor indicated NLuc reassembly was occurring in target cell nuclei upon RNA-guided binding of the MUC4 repetitive region. We then tested our biosensor on anon-repetitive portion of the human MUC4 locus. Comparing our biosensing condition with gRNA to our undirected auto-association condition without gRNA in HeLa cells, normalized signal ranges were 0.96-21.31 RLU/RFU and 0.42-1.76 RLU/RFU, respectively. Average normalized signals were 6.53 RLU/RFU and 0.83 RLU/RFU for the same two conditions, respectively. This is significant difference by unpaired student's t-test on group means (p<0.0001, two-tailed). Furthermore, comparing biosensing conditions to background auto-association conditions in 293T cells, normalized signal ranges were 31.59-1142.48 RLU/RFU and 26.4-53.64 RLU/RFU, respectively. Average normalized signals were 213.77 RLU/RFU and 37.01 RLU/RFU for the same two conditions, respectively. Again, this is a significant difference by unpaired student's t-test on group means (p<0.01, two-tailed). Thus, it was apparent that the biosensor's detection of endogenous level copy number sequences was reliable and consistent and further probing of its sensitivity was warranted.

One pertinent application for this dimeric probe that we imagined would require high sensitivity was isolation of mutant cells from a population of cells after genome editing. To investigate the feasibility of this application, we conducted CRISPR-Cas9 editing experiments at two genomic loci in HCT116 and HEK 293 cells with the goal of using our dimeric biosensor to detect the difference in copy number of a specific sequence between wild-type and homozygous mutant cells. Using difference in signal-to-noise as a primary endpoint, we found that signal-to-noise was higher across several sites bound by gRNA pairs around the original Cas9 cut site in wild-type HEK 293 cells compared to HEK 293 cells that were homozygous mutants for a single-base pair change in the PAM site of the editing gRNA target sequence. This effectively demonstrated differentiation between binding two and zero copies of the target sequence, as HEK 293 cells have two copies of chromosome 16 with no commonly reported abnormalities (41). In HCT116 cells, only one gRNA with overlapping protospacer sequences with PAM sites 28 bp apart showed reliable detection of the target sequence. We hypothesized that mutating the PAM site in both cell lines would create a condition where Cas9 would not be able to recognize the original target site (42). The fact that all gRNA pairs showed higher signal-to-noise in wild-type compared to mutant HEK 293 cells yet this seemingly gRNA-independent effect was not observed in HCT116 cells may be due to intrinsic differences in chromatin structure between cell lines at the edited loci. If this is the case, then future experiments using this biosensor should be planned on the basis of facilitating interactions with more ideal orientation and spacing of DNA target sites given biosensor component orientations. This design strategy makes sense given signal-to-noise was shown to be highly dependent on configuration and phase of the DNA target sites and steric effects between biosensor fusion protein components.

Considering these lines of evidence showing our biosensor rapidly and sensitively detects the presence of specific exogenous and endogenous DNA sequences and changes therein at approximately 2.5-fold to 27-fold above background in live cells, we conclude that it may serve as a very useful platform for many live cell DNA biosensing applications. Moreover, seeing as we also tested our RNP-based biosensor in vitro, which has been a recent focus of many research efforts with the advent of SHERLOCK and other related techniques (42-43), it could even be applicable to the same target market, which currently has a distinct need for rapid, sensitive DNA detection in clinical biosensing of pathogenic DNA sequences. Furthermore, fluorescent amplification of the baseline luminescent signal of the biosensor could be imagined through several routes, which would theoretically increase sensitivity. Further applications could range from expeditious live cell genotyping to detection of interactions between chromatin in three-dimensional space—the magnitude of the scope of possibilities is remarkable.

### Methods

Construction of Directional dCas9-NanoBiT and dCas9-NanoLuc Fusion Proteins

The directional fusion constructs containing the LgBiT and SmBiT of NLuc (Promega Corporation) fused to catalytically inactive Cas9 (D10A and H840A double mutant) were generated using the Gibson Assembly method (New England Biolabs). We used an improved version of the pCDNA3-dCas9 containing two nuclear localization signals, an N-terminal 3× Flag epitope tag and [(GGS)5 (SEQ ID NO: 5)] flexible linker sequences and well as two separate multiple cloning sites at the N- and C-termini of dCas9 (vector map in Supplementary Methods 1, FIG. 11). The LgBiT and SmBiT were each cloned onto the N- and C-termini of dCas9 using two separate multiple cloning sites in the modified pCDNA3-dCas9 vector (see Supplementary Methods 1 for sequences). Overnight N- and C-terminal double restriction digests of sets of flanking restriction sites Xbal and Kpnl and Nhel and Notl, respectively, produced the necessary vector backbones for subsequent Gibson Assembly. LgBiT and SmBiT inserts were ordered as gBlocks Gene Fragments (Integrated DNA Technologies) containing approximately 45 bp homologous sequences with the doubly-digested dCas9 vectors upstream and downstream of the two cut sites. A positive control NLuc-dCas9 fusion construct was created using overlap extension PCR on LgBiT-dCas9 and SmBiT-dCas9 gBlocks to directionally splice the sequences followed by the Gibson Assembly method again using the N-terminal doubly digested dCas9 vector. The four assembled dCas9-NanoBiT constructs, the dCas9-Full NanoLuc construct, and pGL4.53 [luc2/PGK] Firefly luciferase vector (Promega Corporation) were separately transformed into 5-alpha Competent E. coli (New England Biolabs) using a standard chemical transformation procedure with heat shock at 42° C. and transformed E. coli were plated on LB plates containing ampicillin at a final concentration of 100 μg/mL. After an 18-hour incubation at 37° C., MiniPreps (QIAGEN) were created for a subset of large, well-separated colonies. The selected subset of large colonies was screened for recombinant vector and insert using both diagnostic restriction digests and colony PCR. Clones positive for the four NanoBiT inserts, the full NanoLuc insert, and the luc2 insert using both methods were subsequently sequenced to confirm exact sequences were present.

Construction of gRNA Expression Plasmids

The gRNA expression vector backbone was obtained from Addgene (Addgene #41824) and was linearized using a restriction digest with AflII. Two 19-bp gRNA target sequences common throughout several genomes but not present in the human genome were selected using CRISPRscan and the UCSC genome browser (see Example 2, Supplementary Methods 2 for sequences). Each gRNA sequence was incorporated into two 60mer oligonucleotides that contained homologous sequences to the gRNA expression vector for subsequent Gibson assembly. After oligonucleotide annealing and extension, the PCR-purified (PCR purification kit; QIAGEN) 100 bp dsDNA was inserted into the AflII linearized gRNA expression vector using Gibson assembly.

Construction of gRNA Target Site Vector Scaffolds

Scaffolds containing the two gRNA target sequences in tandem, inverted, and everted orientations were created using two separate plans. The first plan consisted of a series of overlap extension PCRs on ssDNA oligonucleotides (Integrated DNA Technologies) followed by PCR purification using the MinElute PCR Purification Kit (QIAGEN). The resulting target sequence scaffold oligonucleotides were then subjected to a final amplification with 2×GoTaq Green Master Mix (Promega Corporation) to create poly-dT tails and cloned into the PCR4TOPO vector using the Topo TA Cloning Kit for Sequencing (Invitrogen). The second plan consisted of a series of targeted blunt-end double restriction digests on cloned scaffolds from the first plan, PCR-purification (removing oligonucleotides<−70 bp) again using the MinElute PCR-purification kit (QIAGEN), and re-ligation using excess T4 DNA ligase (New England Biolabs). See Example 2, Supplementary Methods 3 for sequences.

### Plasmid-Based DNA Biosensor Testing in Live HEK 293T Cells

In the first experiment, which sought to determine the optimal molar transfection ratio of LgBiT to SmBiT fusion constructs, 25,000 low-passage HEK 293T cells per well were seeded in 66 wells of a 96-well white opaque-side microplates (Thermo Fisher Scientific) approximately 20 hours before transfection. These cells were then transiently transfected with 100 ng total DNA per well using the Lipofectamine 3000 transient transfection protocol

(Invitrogen). Each well was transfected with 16.67 ng/well of plasmid expressing each dCas9-NanoBit fusion construct, 16.67 ng/well of plasmid expressing each of two gRNAs, 16.67 ng/well of plasmids containing the target sequence, and 16.67 ng/well pMAX-GFP plasmid as a normalization control for transfection efficiency, cell count, and cell viability. We tested LgBiT:SmBiT molar transfection ratios of 1:50, 1:10, 1:4, 1:2, 1:1.33, 1:1, 1.33:1, 2:1, 4:1, 10:1, and 50:1, the construct in excess being transfected at 16.67 ng/well and the lesser construct being decreased to specific ng amounts based on molar amounts of each of the differently sized constructs. 33 of the LgBiT +SmBiT wells were transfected with the tandem PAMs 10 bp apart target sequence scaffold and 33 of the LgBiT +SmBiT wells were identically transfected but without any target DNA. For wells that did not reach 100 ng total DNA, pUC19 vector was transfected to make up the difference. In this experiment, signals were measured 24 hours post-transfection. In our next experiment, several molar excesses of gRNA to dCas9-NanoBiT fusion constructs (1:1, 1.2:1, 2:1, 5:1, and 20:1) were delivered to cells using the same method as described above, holding the molar amount of gRNA constant but decreasing the molar amount of dCas9-NanoBiT fusion proteins. We then held the 20-fold molar excess gRNA parameter constant and progressively decreased the amount of target DNA transfected, making up the difference with pGL4.53 [luc2/PGK] Firefly luciferase vector (Promega Corporation), essentially random DNA with no binding sites with >5 bp homology with the protospacer of either gRNA. All fluorescent signals were measured on the SpectraMax M5 Microplate Reader (Molecular Devices) with high PMT sensitivity setting and 100 reads/well before taking any luminescent readings. After adding 25 μL furimazine substrate (Promega Corporation) reconstituted at a 1:19 volumetric ratio with Nano-Glo LCS Dilution Buffer (Promega Corporation) according to the Nano-Glo Live Cell Assay System protocol to each well, luminescent signals were measured on the SpectraMax M5 Microplate Reader with 1 sec integration and high PMT sensitivity setting. The ideal delivery parameters were used with the same Lipofectamine 3000 transfection protocol for comparing all orientations of PAM orientation, spacer length, and dCas9-NanoBiT fusion construct pairing.

Production and Purification of Fusion Proteins and gRNAs

We transfected five 50-70% confluent 10 cm plates of low-passage HEK 293T cells with 14 μg total DNA (7 μg fusion construct, 7 μg pMAX-GFP) for each of the five directional dCas9-NanoBiT/Luc fusion constructs using Lipofectamine 3000 (Invitrogen). 24 hours post-transfection, GFP was measured at 50-80% on the EVOS FL Auto 2 fluorescence microscope (Thermo Fisher), indicating a successful, high-efficiency transfection. 48 hours post-transfection, we rinsed the cell pellets twice with 1×phosphate-buffered saline (Invitrogen) and extracted total protein by adding 1 mL 1×RIPA buffer (Cell Signaling Technology) supplemented with 1×protease-phosphatase inhibitor cocktail (Cell Signaling Technology) to cell pellets for 15 minutes followed by sonication (three 2 second pulses with 1 minute on ice between each). Following this, the protein extractions were further incubated on ice for 15 minutes and spun for 10 minutes at 3000 RPM at 4° C. To purify the fusion proteins, we used HA and 3×-Flag immunoprecipitation. C-terminal fusion constructs contained the 3×-Flag epitope and N-terminal fusion constructs contained the HA epitope, so were purified accordingly. We first prepared elution buffers consisting of 3×Flag peptide (Sigma-Aldrich) and HA peptide (Sigma-Aldrich) at 400 μg/mL concentration in a base buffer (50 mM Tris-HCl, 50 mM NaCl, 1 mM EDTA, pH 8.0) for competitive binding in the elution step. Next, we prepared 1×Tris-Buffered Saline (50 mM Tris-HC1, 150 mM NaCl, pH 7.5) and 0.1 M glycine (pH 2.75) for use in wash steps. We first centrifuged AFC-101 P-1000 Mono-HA.11 Affinity Matrix (Covance) and Anti-FLAG M2 Affinity Gel (Sigma-Aldrich) at 8000 g for 1 minute to remove glycerol, then equilibrated both matrices by washing 3 times with 1×TBS. We briefly washed the affinity matrices with 1 mL 0.1 M glycine to ensure an entirely unbound state. This was followed by three more washes with 1×TBS. The extracted total protein supernatants were then added to the appropriate equilibrated matrices and rocked at 4° C. overnight to facilitate fusion protein binding to the matrix. The next morning, bound proteins were eluted by centrifugation of the matrix-protein extract mixtures for 1 minute at 8000 g, three more washes with 1×TBS, and rocking overnight in 200 μL appropriate elution buffer. Expected fusion protein sizes and concentrations were confirmed by native PAGE followed by Western Blot for HA- and 3×-Flag-tagged dCas9-NanoBiT fusion proteins. Purified protein concentrations were also validated using the “Protein A280” setting on the NanoDrop 2000 Spectrophotometer using Beer's Law with molar absorption coefficients calculated for each fusion protein based on tryptophan, tyrosine, and cysteine frequency by formula ε=(5500(nW)+1490(nY)+125(nC)) with 1 cm path length. We concurrently produced gRNAs by in vitro transcription (IVT) using the MEGAscript T7 High Yield Transcription Kit (Ambion). gRNAs were produced from their respective linearized gRNA expression plasmid templates using a 4-hour in vitro T7 RNA Polymerase transcription reaction and purified using phenol-chloroform extraction followed by ethanol precipitation. Correct gRNA size was confirmed on a denaturing TAE agarose gel (See Example 2, Supplementary Methods 4).

### RNP-Based DNA Biosensor Testing in Live HEK 293T Cells

Purified dCas9-NanoBiT/Luc fusion proteins and gRNAs were complexed at 1:1, 1:1.2, 1:2, and 1:3 molar ratios in 25 μL 20 mM HEPES with 150 mM KC1 (pH 7.5) with target DNA and mixed with 25 μL reconstituted furimazine substrate (Promega Corporation) in 96-well white opaque-side microplates (Thermo Fisher Scientific) to confirm the ribonucleoprotein complexes were active by observing NanoLuc signal production. NanoLuc luminescent signals were then measured on the SpectraMax M5 Microplate Reader (Molecular Devices) 50 minutes, 100 minutes, 150 minutes, and 200 minutes after complexation. In live cell assays, dCas9-NanoBiT/Luc RNPs were complexed and delivered to cells using a method purported to result in increased cleavage efficiencies in knockout assays, Lipofectamine CRISPRMAX (Invitrogen). Target DNA and a recombinant GFP (Abeam) transfection control were co-delivered with RNPs by addition to the Lipofectamine CRISPRMAX RNP mixture after a 10-minute complexation time. In the first experiment, we varied the amount of the LgBiT-dCas9 fusion protein from 105 ng to 25 ng while adding dCas9-SmBiT at 4-fold and 10-fold molar excesses. All tests were conducted on target site scaffolds with tandem target sites 10 bp apart and with inverted target sites 15 bp apart in this experiment. In the next experiment where 12 different target site scaffolds were tested, 105 ng of dCas9-SmBiT was delivered in 4-fold and 10-fold molar excesses to LgBiT-dCas9. LgBiT-dCas9 and dCas9-SmBiT were delivered in these experiments as negative controls and NLuc-dCas9 was delivered as a positive control. In the experiments testing response of the NLuc signal to decreasing target DNA concentration, 100-n ng pGL4.53 [luc2/PGK] Firefly luciferase vector (Promega Corporation), essentially random DNA of approximately the same size with no binding sites with >5 bp homology with the protospacer of either gRNA was added to the transfection mix in conditions where a ng amount (n) of target sequence scaffold was subtracted from the original 100 ng.

### Luminescence Microscopy and Image Processing

Transfection experimental setup for microscopy sessions was identical to the setup for microplate reader sessions. In these experiments, low-passage HEK 293T cells were plated in SensoPlate 24 Well F-Bottom, Glass Bottom Black Microplates (Greiner Bio-One) and transfected identically to luminometer-based experiments. Instead of imaging whole well populations of adherent cells, we split the cells to 1.5×10⁵ cells/mL and took images of the cell suspensions on Superfrost Plus Microscope Slides (Fisher Scientific) with Premium Cover Glass (Fisher Scientific). An optimized NLuc imaging protocol was developed for use on the Leica DM6000 B Fully Automated Upright Microscope equipped with the Leica DFC9000 GT sCMOS camera and the Exfo X-Cite 120 Fluorescence Illumination System in which cells were placed in a dark box with all light sources covered or off and lamp intensity was set to 0, exposure time was set to 30 s, and sCMOS gain was set to 2.0. The pMAX-GFP transfection normalization control was imaged using an exposure of 150 ms and sCMOS gain of 1.0. The WEKA Segmentation package (44) in Fiji (Image J) was used to delineate boundaries of cell nuclei and then integrate signal intensities within these regions after several training cycles. Raw 16-bit grayscale GFP images were recolored green, brightness was reduced, and contrast was enhanced in Fiji. Raw 16-bit grayscale NLuc images were recolored magenta, brightness and contrast were increased, and the “remove outliers” and “despeckle” noise reduction functions were applied in Fiji (Image J). Following this, scattered speckled noise remained in these images, so the noise was carefully removed around the cell nuclear regions in the GNU Image Manipulation Program (GIMP) using the clone tool with radius 5.0. To merge GFP and NLuc images, we took one of two routes: we either directly merged color channels in Fiji (Image J), or if the NLuc signal was drowned out by the merge due to its disproportionate dimness, the two separate images were opened in GIMP, making the processed NLuc image the upper layer. Then, opacity of the NLuc layer was reduced to approximately 95% in order to visualize the NLuc signal.

### IVIS Spectrum Imaging

For RNP-based experiments on the IVIS Spectrum Bioluminescence Imaging System, we again split cells to 1.5×10⁵ cells/mL but suspended them in 7.5 mL Opti-MEM Reduced Serum Medium (Fisher Scientific) on 100 mm Polystyrene Petri Dishes (Fisher Scientific). We developed an optimized imaging protocol on the IVIS using field of view C (FOV C=13.3 cm), 0 cm specimen height, medium binning, F/Stop of 1, excitation filter set to “block,” emission filter set to “open,” and exposure set to “auto.” Within the LivingImage software associated with the IVIS Spectrum, we adjusted the scale of all images to be equal and compared signal-to-noise ratios by drawing and integrating circular regions of interest (ROIs) around regions containing cell nuclei as judged by presence of luminescent signal. Negative controls in initial IVIS experiments using target site scaffold vectors were cells without target DNA transfected.

### Statistical Testing

Two-tailed student's t-tests and Z-tests for signal-to-noise analyses were conducted in Microsoft Excel 2016. Two-way ANOVA and pairwise Tukey's HSD post-hoc tests were conducted in R on combinatorial signals from our initial biosensing experiments in live cells.

### References

1. Giuliano, C. J., Lin, A., Girish, V. & Sheltzer, J. Generating single cell-derived knockout clones in mammalian cells with CRISPR/Cas9. Current Protocols in Molecular Biology 128, e100 (2019).
2. Mathupala, S. & Sloan, A. A. An agarose-based cloning-ring anchoring method for isolation of viable cell clones. BioTechniques 46, 305-307 (2009).
3. Hu, P., Wenhua Zhang, Xin, H., and Deng, G. Single cell isolation and analysis. Frontiers in Cell and Developmental Biology 4, 116 (2016).
4. Sentmanat, M. F., Peters, S. T., Florian, C. P., Connelly, J. P. & Pruett-Miller, S. M. A survey of validation strategies for CRISPR-Cas9 editing. Scientific Reports 8, 888 (2018).
5. Ren, C., Xu, K., Segal, D. J. & and Zhang, Z. Strategies for the enrichment and selection of genetically modified cells. Trends in Biotechnology 37, 56-71 (2019).
6. Bauer, D. E., Canver, M. C. & Orkin, S. H. Generation of genomic deletions in mammalian cell lines via CRISPR/Cas9. Journal of Visualized Experiments: JoVE, 95 e52118 (2015).
7. Vouillot, L., Thélie, A., and Pollet, N. Comparison of T7E1 and surveyor mismatch cleavage assays to detect mutations triggered by engineered nucleases. G3 5, 407-15 (2015).
8. Zotova, A. et al. “Isolation of gene-edited cells via knock-in of short glycophosphatidylinositol-anchored epitope tags.” Scientific Reports 9, 3132 (2019).
9. Li, X. et al. Highly efficient genome editing via CRISPR-Cas9 in human pluripotent stem cells is achieved by transient BCL-XL overexpression. Nucleic Acids Research 46, 10195-215 (2018).
10. Tamm, C., Kadekar, S., Pijuan-Galitó, S. & Annerén, C. Fast and efficient transfection of mouse embryonic stem cells using non-viral reagents. Stem Cell Reviews 12, 584-91 (2016).
11. Zhang, Z. et al. CRISPR/Cas9 genome-editing system in human stem cells: current status and future prospects. Molecular Therapy. Nucleic Acids 9, 230-41 (2017).
12. Bruenker, H-G. 558. High efficiency transfection of primary cells for basic research and gene therapy. Molecular Therapy: The Journal of the American Society of Gene Therapy 13, 5215 (2006).
13. Modarai, S. R. et al. Efficient delivery and nuclear uptake is not sufficient to detect gene editing in CD34+cells directed by a ribonucleoprotein complex. Molecular Therapy. Nucleic Acids 11, 116-29 (2018).
14. Liu, M. et al. Methodologies for improving HDR efficiency. Frontiers in Genetics 9, 691 (2019).
15. Chen, B. et al. Dynamic imaging of genomic loci in living human cells by an optimized CRISPR/Cas system. Cell 155, 1479-91 (2013).
16. Ye, H., Rong, Z., and Lin, Y. Live cell imaging of genomic loci using dCas9-SunTag system and a bright fluorescent protein. Protein & Cell 8, 853-55 (2017).
17. Chen, B., Zou, W., Xu, H., Liang, Y. & Huang, B. Efficient labeling and imaging of protein-coding genes in living cells using CRISPR-Tag.” Nature Communications 9, 5065 (2018).
18. Dreissig, S. et al. Live-cell CRISPR imaging in plants reveals dynamic telomere movements. The Plant Journal: For Cell and Molecular Biology 91, 565-73 (2017).
19. Wu, X., Mao, S., Ying, Y., Krueger, C. J. & Chen, A. K. Progress and challenges for live-cell imaging of genomic loci using CRISPR-based platforms. Genomics, Proteomics & Bioinformatics 17, 119-128 (2019).
20. Deng, W., Shi, X., Tjian, R., Lionnet, T. & Singer, R. H. CASFISH: CRISPR/Cas9-mediated in situ labeling of genomic loci in fixed cells. Proceedings of the National Academy of Sciences of the United States of America 112, 11870-75 (2015).
21. Zhang, D. et al. CRISPR-Bind: A simple, custom CRISPR/dCas9-mediated labeling of genomic DNA for mapping in nanochannel arrays. bioRxiv (2018).
22. Ma, H. et al. Multicolor CRISPR labeling of chromosomal loci in human cells. Proceedings of the National Academy of Sciences of the United States of America 112, 3002-7 (2015).
23. Boutorine, A. S., Novopashina, D. S., Krasheninina, 0. A., Nozeret, K. & Venyaminova, A. G. Fluorescent probes for nucleic acid visualization in fixed and live cells. Molecules 18, 15357-97 (2013).
24. Dahan, L., Huang, L., Kedmi, R., Behlke, M. A. & Peer, D. SNP detection in mRNA in living cells using allele specific FRET probes.” PloS One 8, e72389 (2013).
25. Didenko, V. V. DNA probes using fluorescence resonance energy transfer (FRET): designs and applications. BioTechniques 31, 1106-16, 1118, 1120-21 (2001).
26. Wu, X., et al. A CRISPR/molecular beacon hybrid system for live-cell genomic imaging. Nucleic Acids Research 46, e80 (2018).
27. Mao, S., Ying, Y., Wu, X., Krueger, C. J. & Chen, A. K. CRISPR/dual-FRET molecular beacon for sensitive live-cell imaging of non-repetitive genomic loci. Nucleic Acids Research gkz752 (2019).
28. Stains, C. I., Porter, J. R., Ooi, A.T., Segal, D. J. & Ghosh, I. DNA sequence-enabled reassembly of the green fluorescent protein. Journal of the American Chemical Society 127, 10782-83 (2005).
29. Ooi, A. T., Stains, C. I., Ghosh, I. & Segal, D. J. Sequence-enabled reassembly of beta-lactamase (SEER-LAC): a sensitive method for the detection of double-stranded DNA. Biochemistry 45, 3620-25 (2006).
30. Ghosh, I., Stains, C. I., Ooi, A. T. & Segal, D. J. Direct detection of double-stranded DNA: molecular methods and applications for DNA diagnostics.” Molecular bioSystems 2, 551-60 (2006).
31. Zhang, Y. et al. Paired design of dCas9 as a systematic platform for the detection of featured nucleic acid sequences in pathogenic strains. ACS Synthetic Biology 6, 211-16 (2017).
32. Bernas, T., Robinson, J. P., Asem, E. K. & Rajwa, B. Loss of image quality in photobleaching during microscopic imaging of fluorescent probes bound to chromatin. Journal of Biomedical Optics 10, 064015 (2005).
33. Tung, J. K., Berglund, K., Gutekunst, C., Hochgeschwender, U. & Gross, R. E. Bioluminescence imaging in live cells and animals. Neurophotonics 3, 025001 (2016).
34. Cook, E., Hermes, J., Li, J. & Tudor, M. High-content reporter assays. Methods in Molecular Biology 1755, 179-95 (2018).
35. Choy, G. et al. Comparison of Noninvasive Fluorescent and Bioluminescent Small Animal Optical Imaging.” BioTechniques (2003). https://doi.org/10.2144/03355a02.
36. Hall, M. P. et al. Engineered luciferase reporter from a deep sea shrimp utilizing a novel imidazopyrazinone substrate. ACS Chemical Biology 7, 1848-57 (2012).
37. England, C. G., Ehlerding, E. B. & Cai, W. NanoLuc: a small luciferase is brightening up the field of bioluminescence. Bioconjugate Chemistry 27, 1175-87 (2016).
38. Dixon, A. S. et al. NanoLuc complementation reporter optimized for accurate measurement of protein interactions in cells. ACS Chemical Biology 11, 400-408 (2016).
39. Coggins, N. B., Stultz, J., O'Geen, H., Carvajal-Carmona, L. G. & Segal, D. J. Methods for scarless, selection-free generation of human cells and allele-specific functional analysis of disease-associated SNPs and variants of uncertain significance. Nature Scientific Reports 7, 15044 (2017).
40. Schwanhäusser, B. et al. Global quantification of mammalian gene expression control. Nature 473, 337-42 (2011).
41. Lin, Y., et al. Genome dynamics of the human embryonic kidney 293 lineage in response to cell biology manipulations. Nature Communications 5, article number 4767 (2014).
42. Jiang, F. & Doudna, J.A. CRISPR—Cas9 structures and mechanisms. Annual Review of Biophysics 46, 505-29 (2017).
43. Gootenberg, J. S. et al. Multiplexed and portable nucleic acid detection platform with cas13, casl2a, and csm6. Science 360, 439-44 (2018).
44. Li, S. et al. CRISPR-cas12a-assisted nucleic acid detection. Cell Discovery 4, 1-4 (2018).
45. Arganda-Carreras, I. et al. Trainable weka segmentation: a machine learning tool for microscopy pixel classification. Bioinformatics 33, 2424-26 (2017).

### Example 2: Supplementary Methods, Tables and Sequences

TABLE 1
Biosensor signal output variability across seven individual non-
repetitive loci at MUC4.

| #Two-way ANOVA | | | | |
|---|---|---|---|---|
| summary | Df | Sum sq | F value | Pr(>F) |
| Fusion Protein Orientation | 3 | 28557 | 302.92 | <2e−16 |
| Target DNA Orientation | 32 | 1498 | 1.49 | 0.0494 |
| FP Orientation:DNA | 96 | 6227 | 2.06 | 2.94E−06 |
| Orientation | | | | |
| | 264 | 8296 | | |
| Signif. Codes | 0.001 = | 0.01 = '**' | 0.05 = '*' | |
| | ***** | | | |

TABLE 2
Biosensor signal output variability across seven individual
non-repetitive loci at MUC4.

| | diff | lwr | upr | p adj |
|---|---|---|---|---|
| LCSN-LCSC | −0.9021779 | −2.9621672 | 1.15781142 | 0.66988987 |
| LNSC-LCSC | 16.3056095 | 14.2456202 | 18.3655988 | 7.57E−14 |
| LNSN-LCSC | −6.4615939 | −8.5215832 | −4.4016046 | 2.33E−13 |
| LNSC-LCSN | 17.2077874 | 15.1477981 | 19.2677767 | 7.57E−14 |
| LNSN-LCSN | −5.559416 | −7.6194053 | −3.4994267 | 1.45E−10 |
| LNSN-LNSC | −22.767203 | −24.827193 | −20.707214 | 7.57E−14 |

### Supplementary Methods 1: Process for Creation of dCas9-NanoBiT Fusion Constructs gBlocks for Initial dCas9-NanoBiT Cloning Scheme

### NLS-HA-LgBiT (Nfus):

(SEQ ID NO: 6)
TCCATAGAAGACACCGGGACCGATCCAGCCTCCGGACTCTAGAGGATCG
AACCCTTGCCACCATGCCCAAGAAGAAGAGGAAGGTGGGAGGCTCCGGA
GGAAGCTACCCATACGATGTCCCAGACTACGCGGGTGGCGGGTCCGGCG
GTGGATCCATGGTCTTCACACTCGAAGATTTCGTTGGGGACTGGGAACA
GACAGCCGCCTACAACCTGGACCAAGTCCTTGAACAGGGAGGTGTGTCC
AGTTTGCTGCAGAATCTCGCCGTGTCCGTAACTCCGATCCAAAGGATTG
TCCGGAGCGGTGAAAATGCCCTGAAGATCGACATCCATGTCATCATCCC
GTATGAAGGTCTGAGCGCCGACCAAATGGCCCAGATCGAAGAGGTGTTT
AAGGTGGTGTACCCTGTGGATGATCATCACTTTAAGGTGATCCTGCCCT
ATGGCACACTGGTAATCGACGGGGTTACGCCGAACATGCTGAACTATTT
CGGACGGCCGTATGAAGGCATCGCCGTGTTCGACGGCAAAAAGATCACT
GTAACAGGGACCCTGTGGAACGGCAACAAAATTATCGACGAGCGCCTGA
TCACCCCCGACGGCTCCATGCTGTTCCGAGTAACCATCAACAGTGGTAC
CGGAGGGAGTGGTGGAAGCGGCGGTTCTGGTGGCTCAG

Assemble into Xbal, Kpnl doubly-digested iCas9 V3 vector by Gibson assembly.

### NLS-HA-SmBiT (Nfus):

(SEQ ID NO: 7)
TCCATAGAAGACACCGGGACCGATCCAGCCTCCGGACTCTAGAGGATCG
AACCCTTGCCACCATGCCCAAGAAGAAGAGGAAGGTGGGAGGCTCCGGA
GGAAGCTACCCATACGATGTCCCAGACTACGCGGGTGGCGGGTCCGGCG
GTGGATCCATGGTGACCGGCTACCGGCTGTTCGAGGAGATTCTCGGTAC
CGGAGGGAGTGGTGGAAGCGGCGGTTCTGGTGGCTCAG

Assemble into Xbal, Kpnl doubly-digested iCas9 V3 vector by Gibson assembly.

### LgBiT-NLS (Cfus):

(SEQ ID NO: 8)
TAGTGGAGGTTCAGGAGGATCCGGGGGGAGCGGAGGGAGCGCTAGCGTC
TTCACACTCGAAGATTTCGTTGGGGACTGGGAACAGACAGCCGCCTACA
ACCTGGACCAAGTCCTTGAACAGGGAGGTGTGTCCAGTTTGCTGCAGAA
TCTCGCCGTGTCCGTAACTCCGATCCAAAGGATTGTCCGGAGCGGTGAA
AATGCCCTGAAGATCGACATCCATGTCATCATCCCGTATGAAGGTCTGA
GCGCCGACCAAATGGCCCAGATCGAAGAGGTGTTTAAGGTGGTGTACCC
TGTGGATGATCATCACTTTAAGGTGATCCTGCCCTATGGCACACTGGTA
ATCGACGGGGTTACGCCGAACATGCTGAACTATTTCGGACGGCCGTATG
AAGGCATCGCCGTGTTCGACGGCAAAAAGATCACTGTAACAGGGACCCT
GTGGAACGGCAACAAAATTATCGACGAGCGCCTGATCACCCCCGACGGC
TCCATGCTGTTCCGAGTAACCATCAACAGCGGTGGAGGCTCCGGAGGTG
GATCTAAAAGGCCGGCGGCCACGAAAAAGGCCGGTCAGGCAAAAAAGAA
AAAGGGTGGTAGTGGAAGCGGAGCGGCCGCATGAAAGGGTTCGATCCCT
ACCGGTTAGTAATGAGT

Assemble into Nhel, Notl doubly-digested iCas9 V3 vector by Gibson assembly.

### SmBiT-NLS (Cfus):

(SEQ ID NO: 9)
TAGTGGAGGTTCAGGAGGATCCGGGGGGAGCGGAGGGAGCGCTAGCGTG
ACCGGCTACCGGCTGTTCGAGGAGATTCTGGGTGGAGGCTCCGGAGGTG
GATCTAAAAGGCCGGCGGCCACGAAAAAGGCCGGTCAGGCAAAAAAGAA
AAAGGGTGGTAGTGGAAGCGGAGCGGCCGCATGAAAGGGTTCGATCCCT
ACCGGTTAGTAATGAGT

Assemble into Nhel, Notl doubly-digested iCas9 V3 vector by Gibson assembly. For HC91V3 (iCas9V3) vector map, see FIG. 11.

### Overlap Extension PCR Primers to Create NLuc-dCas9 Fusion Construct

FP 1 (LgBiT-N gBlock):
(SEQ ID NO: 10)
5′-TCCATAGAAGACACCGGGAC
RP 1 (LgBiT-N gBlock w/ 5′ homology to SmBiT-N
gBlock):
(SEQ ID NO: 11)
5'-CGAACAGCCGGTAGCCGGTCACACTGTTGATGGTTACTCGGAAC
FP 2 (SmBiT-N gBlock w/ 5′ homology to LgBiT-N
gBlock):
(SEQ ID NO: 12)
5′-GTTCCGAGTAACCATCAACAGTGTGACCGGCTACCGGCTGTTCG
RP 2 (SmBiT-N gBlock):
(SEQ ID NO: 13)
5′-CTGAGCCACCAGAACCGCCGC

Final verified protein sequences:

### NLuc-dCas9:

(SEQ ID NO: 14)
MPKKKRKVGGSGGSYPYDVPDYAGGGSGGGSMVFTLEDFVGDWEQTAA
YNLDQVLEQGGVSSLLQNLAVSVTPIQRIVRSGENALKIDIHVIIPYE
GLSADQMAQIEEVFKVVYPVDDHHFKVILPYGTLVIDGVTPNMLNYFG
RPYEGIAVFDGKKITVTGTLWNGNKIIDERLITPDGSMLFRVTINSVT

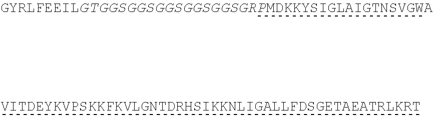

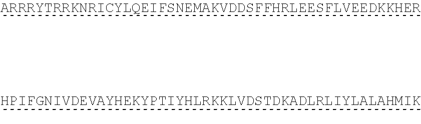

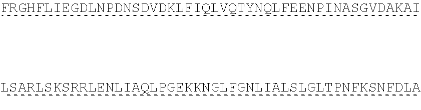

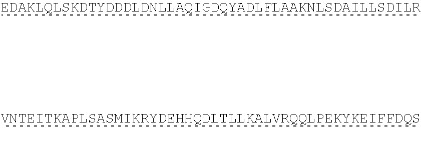

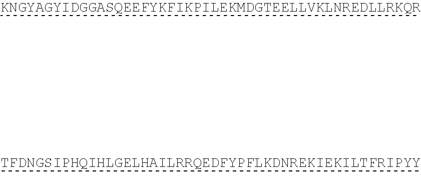

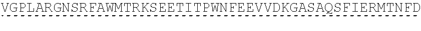

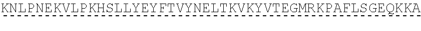

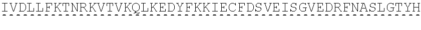

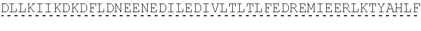

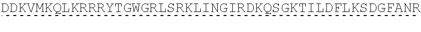

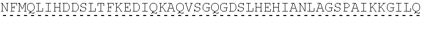

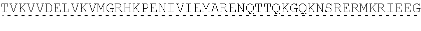

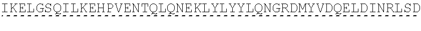

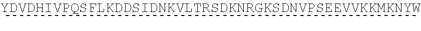

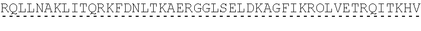

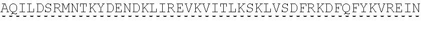

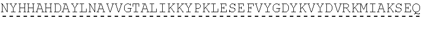

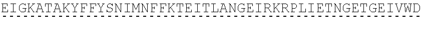

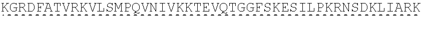

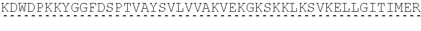

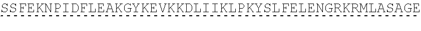

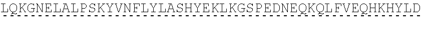

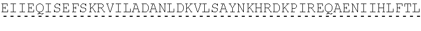

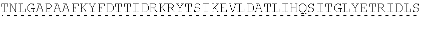

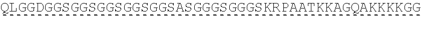

SGSGATNFSLLKQAGDVEENPGPAAA*

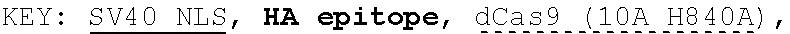

NLuc, Nucleoplasmin NLS, P2A, variable length
flexible linkers

### LgBiT-dCas9 (SEQ ID NO: 1):

MPKKKRKVGGSGGSYPYDVPDYAGGGSGGGSMVFTLEDFVGDWEQTAA
YNLDQVLEQGGVSSLLQNLAVSVTPIQRIVRSGENALKIDIHVIIPYE
GLSADQMAQIEEVFKVVYPVDDHHFKVILPYGTLVIDGVTPNMLNYFG
RPYEGIAVFDGKKITVTGTLWNGNKIIDERLITPDGSMLFRVTINGT

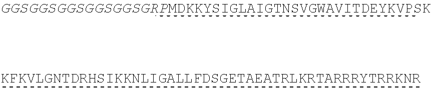

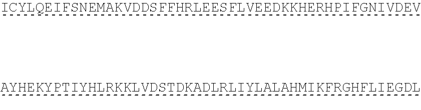

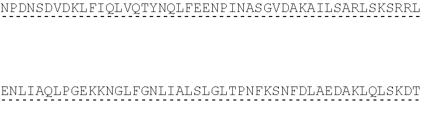

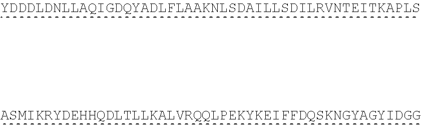

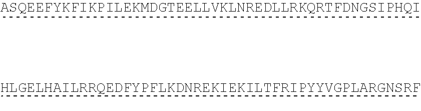

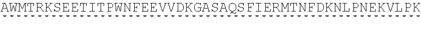

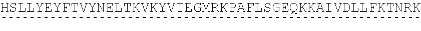

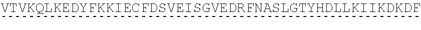

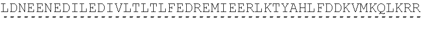

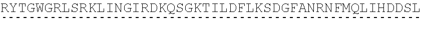

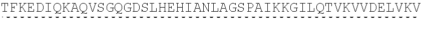

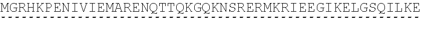

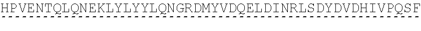

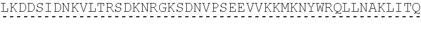

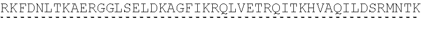

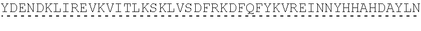

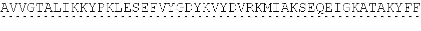

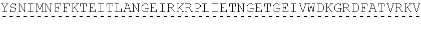

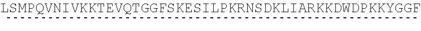

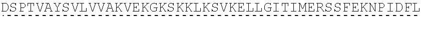

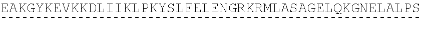

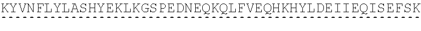

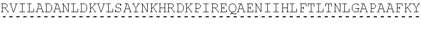

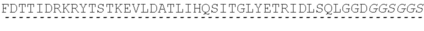

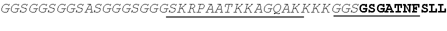

KQAGDVEENPGPAAA*

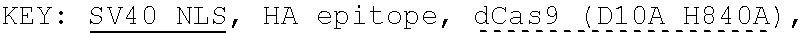

LgBiT, Nucleoplasmin NLS,
P2A, variable length flexible linkers

### SmBiT-dCas9 (SEQ ID NO: 2):

MPKKKRKVGGSGGSYPYDVPDYAGGGSGGGSMVTGYRLFEEILGTGGS

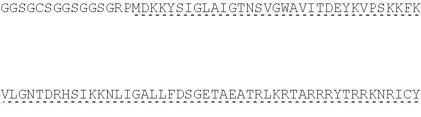

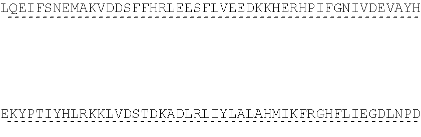

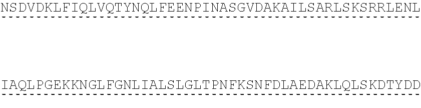

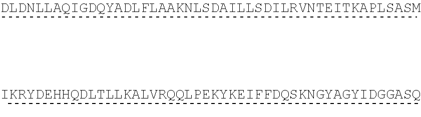

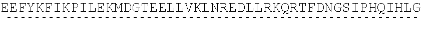

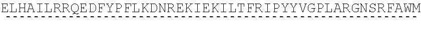

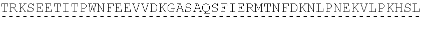

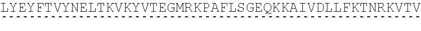

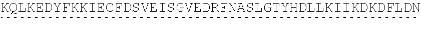

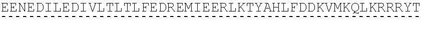

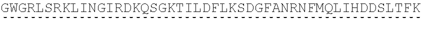

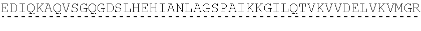

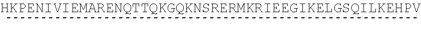

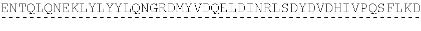

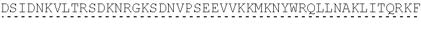

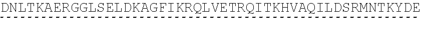

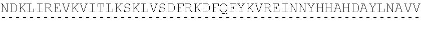

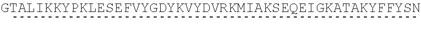

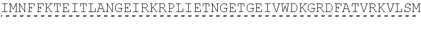

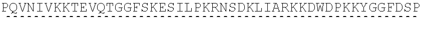

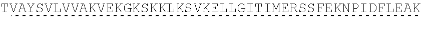

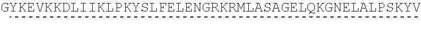

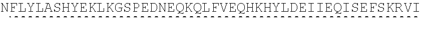

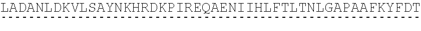

GGSGGSASGGGSGGGSKRPAATKKAGQAKKKKGGSGSGATNFSLLKQA
GDVEENPGPAAA*

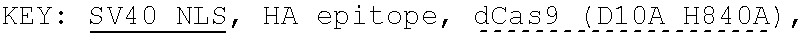

SmBiT, Nucleoplasmin NLS,
P2A, variable length flexible linkers

dCas9-LgBiT (SEQ ID NO: 3):

MPKKKRKVGGSGGSDYKDHDGDYKDHDIDYKDDDDKGGGSGGGSGTGG

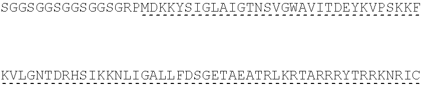

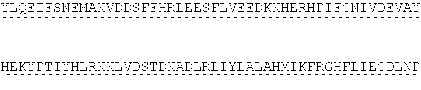

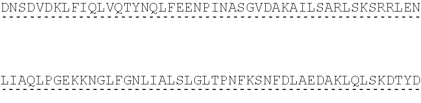

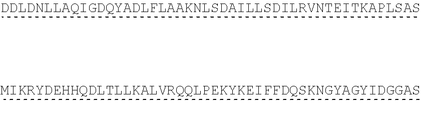

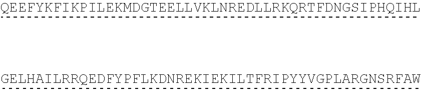

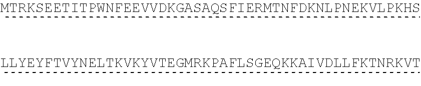

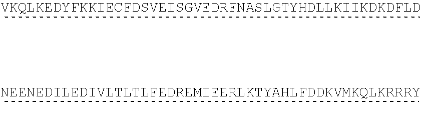

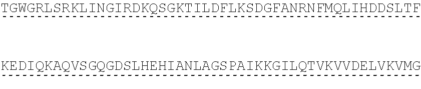

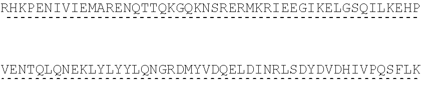

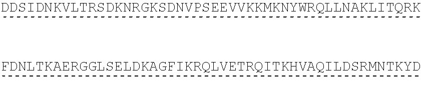

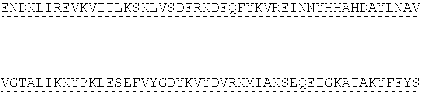

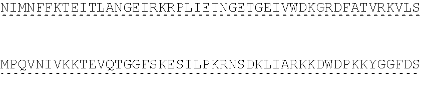

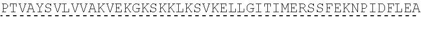

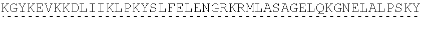

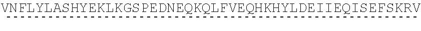

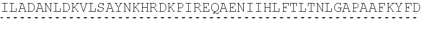

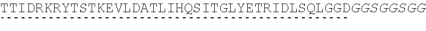

SGGSGGSASVFTLEDFVGDWEQTAAYNLDQVLEQGGVSSLLQNLAVSV
TPIQRIVRSGENALKIDIHVIIPYEGLSADQMAQIEEVFKVVYPVDDH
HFKVILPYGTLVIDGVTPNMLNYFGRPYEGIAVFDGKKITVTGTLWNG
NKIIDERLITPDGSMLFRVTINSGGGSGGGSKRPAATKKAGQAKKKKG
GSGSGAAA*

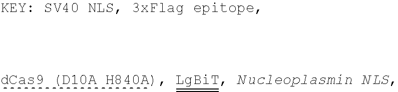

variable length flexible linkers

dCas9-SmBiT (SEQ ID NO: 4):

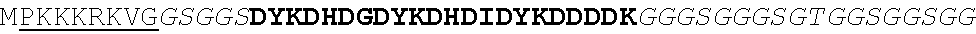

SGGSGGSGRPMDKKYSIGLAIGTNSVGWAVITDEYKVPSKKFKVLGNTDRHSIKKNL
IGALLFDSGETAEATRLKRTARRRYTRRKNRICYLQEIFSNEMAKVDDSFFHRLEESF
LVEEDKKHERHPIFGNIVDEVAYHEKYPTIYHLRKKLVDSTDKADLRLIYLALAHMI
KFRGHFLIEGDLNPDNSDVDKLFIQLVQTYNQLFEENPINASGVDAKAILSARLSKSR
RLENLIAQLPGEKKNGLFGNLIALSLGLTPNFKSNFDLAEDAKLQLSKDTYDDDLDN
LLAQIGDQYADLFLAAKNLSDAILLSDILRVNTEITKAPLSASMIKRYDEHHQDLTLL
KALVRQQLPEKYKEIFFDQSKNGYAGYIDGGASQEEFYKFIKPILEKMDGTEELLVKL
NREDLLRKQRTFDNGSIPHQIHLGELHAILRRQEDFYPFLKDNREKIEKILTFRIPYYV
GPLARGNSRFAWMTRKSEETITPWNFEEVVDKGASAQSFIERMTNFDKNLPNEKVLP
KHSLLYEYFTVYNELTKVKYVTEGMRKPAFLSGEQKKAIVDLLFKTNRKVTVKQLK
EDYFKKIECFDSVEISGVEDRFNASLGTYHDLLKIIKDKDFLDNEENEDILEDIVLTLTL
FEDREMIEERLKTYAHLFDDKVMKQLKRRRYTGWGRLSRKLINGIRDKQSGKTILDF
LKSDGFANRNFMQLIHDDSLTFKEDIQKAQVSGQGDSLHEHIANLAGSPAIKKGILQT
VKVVDELVKVMGRHKPENIVIEMARENQTTQKGQKNSRERMKRIEEGIKELGSQILK
EHPVENTQLQNEKLYLYYLQNGRDMYVDQELDINRLSDYDVDHIVPQSFLKDDSID
NKVLTRSDKNRGKSDNVPSEEVVKKMKNYWRQLLNAKLITQRKFDNLTKAERGGL
SELDKAGFIKRQLVETRQITKHVAQILDSRMNTKYDENDKLIREVKVITLKSKLVSDF
RKDFQFYKVREINNYHHAHDAYLNAVVGTALIKKYPKLESEFVYGDYKVYDVRKM
IAKSEQEIGKATAKYFFYSNIMNFFKTEITLANGEIRKRPLIETNGETGEIVWDKGRDF
ATVRKVLSMPQVNIVKKTEVQTGGFSKESILPKRNSDKLIARKKDWDPKKYGGFDSP
TVAYSVLVVAKVEKGKSKKLKSVKELLGITIMERSSFEKNPIDFLEAKGYKEVKKDLI
IKLPKYSLFELENGRKRMLASAGELQKGNELALPSKYVNFLYLASHYEKLKGSPEDN
EQKQLFVEQHKHYLDEIIEQISEFSKRVILADANLDKVLSAYNKHRDKPIREQAENIIH
LFTLTNLGAPAAFKYFDTTIDRKRYTSTKEVLDATLIHQSITGLYETRIDLSQLGGDG

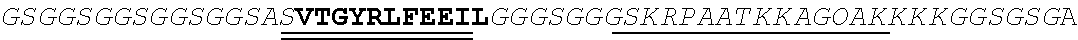

AA*

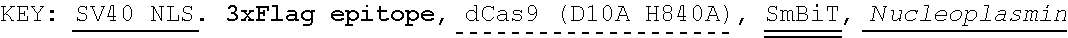

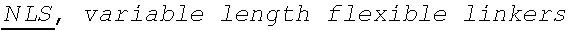

### Supplementary Methods 2: Process for Creation of gRNAs

JL gRNAs oligos for annealing to create Al and J1,2 gRNAs
JL gRNA1

gRNA1 =
(SEQ ID NO: 15)
GCTCCCTACGCATGCGTCCC
DNA target site 1/A (fwd) =
(SEQ ID NO: 16)
GCTCCCTACGCATGCGTCCCAGG

JL gRNA1

gRNA2 =
(SEQ ID NO: 17)
GATGGCTCAGGTTTGTCGCG
DNA target site 2/B (fwd) =
(SEQ ID NO: 18)
GATGGCTCAGGTTTGTCGCGCGG
Insert F:
(SEQ ID NO: 19)
TTTCTTGGCTTTATATATCTTGTGGAAAGGACGAAACACC

[symbol]

[symbol]

Insert R:
(SEQ ID NO: 20)
GAGTAGCCTTATTTTAACTTGCTATTTCTAGCTCTAAAAC

[symbol]

[symbol]

JL gRNA1 F

(SEQ ID NO: 21)
TTTCTTGGCTTTATATATCTTGTGGAAAGGACGAAACACC

[symbol]

[symbol]

JL gRNA1 R

(SEQ ID NO: 22)
GACTAGCCTTATTTTAACTTGCTATTTCTAGCTCTAAAAC

[symbol]

[symbol]

JL gRNA2 F

(SEQ ID NO: 23)
TTTCTTGGCTTTATATATCTTGTGGAAAGGACGAAACACC

[symbol]

[symbol]

JL gRNA2 R

(SEQ ID NO: 24)
GACTAGCCTTATTTTAACTTGCTATTTCTAGCTCTAAAAC

[symbol]

[symbol]

### Supplementary Methods 3: Process for Creation of DNA Target Site Plasmids

gBlock 1 Sequence with tandem A and B target sites:

(SEQ ID NO: 25)
GGGTTTGCTGCTCATCTATACTTTCACAATCTTGAGCTGCAGGGCAAAGA
GCTCCCTACGCATGCGTCCCAGGCAGCGTGTATAGTGAAAAGGAACCCGG
GGATGGAGGAAGGGACATAGGGAGATGGCTCAGGTTTGTCGCGCGGTATG
TAGCATGGCCCGGGAAGTACAGTAGAGCTCCCTACGCATGCGTCCCAGGT
GCTACTTACATATTCTCCCGGGTAAATTAATTCTTATGAGATGGCTCAGG
TTTGTCGCGCGGCTAGTAGCCCGGGGCATTGTGCTCCCTACGCATGCGTC
CCAGGATCTAATCATATCCCGGGATGAAGGTCTATGATGGCTCAGGTTTG
TCGCGCGGTATGCTGAATAATTGAGCCCGGGATAGTGAAATTTATGATGC
TCCCTACGCATGCGTCCCAGGTGCTTTTCCCGGGTGCACAAGATGGCTCA
GGTTTGTCGCGCGGAATATAATAATATGTAGATGGTCCCGGGTAGGTTGT
TATACATTTACTGAGCTCCCTACGCATGCGTCCCAGGTTTGTAGAAGGCT
AGGGGAACAGGTTAGTTTGAGGGAATTCTAATGGATCCTTCTATGGG

PCR and OE-PCR on gBlock 1 to generate spacers from 6 bp to 50 bp. Bold indicates mispriming), [symbol] indicates Target Site A, [symbol] indicates Target Site B

6 bp spacer:

FP1:
(SEQ ID NO: 26)
5′-CTTGAGCTGCAGGGCAAA
(Tm 68) (Taq 58)
RP1:
(SEQ ID NO 27)
5′- [symbol] [symbol] CTGCCTGGGACGCATGC
(Tm 72) (Taq 63)
FP2:
(SEQ ID NO: 28)
5′- [symbol] [symbol] GGAGATGGCTCAGGTTTGTCG
(Tm 70) (Taq 61)
RP2:
(SEQ ID NO: 29)
5′-TTCCCGGGCCATGCTACA
(Tm 72) (Taq 62)

FP1 and RP1 generate 64 bp product, FP2 and RP2 generate 64 bp product.

FP1 and RP2 with round 1 products as templates generate 92 bp product.

### Final seq:

(SEQ ID NO: 30)
5′-CTTGAGCTGCAGGGCAAAGA [symbol] [symbol]
CAGGGA [symbol] [symbol] [symbol]
TATGTAGCATGGCCCGGGAA

10 bp spacer:

FP1:
(SEQ ID NO: 26)
5′-CTTGAGCTGCAGGGCAAA
(Tm 68) (Taq 58)
RP1:
(SEQ ID NO: 31)
5′- [symbol] [symbol] CGCTGCCTGGGACGCAT
(Tm 74) (Taq 65)
FP2:
(SEQ ID NO: 32)
5′- [symbol] [symbol] AGGGAGATGGCTCAGGTTTG
(Tm 68) (Taq 59)
RP2:
(SEQ ID NO: 29)
5′-TTCCCGGGCCATGCTACA
(Tm 72) (Taq 62)

FP1 and RP1 generate 66 bp product, FP2 and RP2 generate 66 bp product.

FP1 and RP2 with round 1 products as templates generate 96 bp product.

### Final seq:

(SEQ ID NO: 33)
5′-
CTTGAGCTGCAGGGCAAAGA [symbol] [symbol]
CAGCGAGGGA [symbol] [symbol] [symbol]
TATGTAGCATGGCCCGGGAA

15 bp spacer:

FP1:
(SEQ ID NO: 26)
5′-CTTGAGCTGCAGGGCAAA
(Tm 68) (Taq 58)
RP1:
(SEQ ID NO: 34)
5′- [symbol] [symbol] ACACGCTGCCTGGGACG
(Tm 72) (Taq 65)
FP2:
(SEQ ID NO: 35)
5′- [symbol] [symbol] ATAGGGAGATGGCTCAGGTTTG
(Tm 67) (Taq 59)
RP2:
(SEQ ID NO: 29)
5′-TTCCCGGGCCATGCTACA
(Tm 72) (Taq 62)

FP1 and RP1 generate 69 bp product, FP2 and RP2 generate 68 bp product.

FP1 and RP2 with round 1 products as templates generate 101 bp product.

### Final seq:

(SEQ ID NO: 36)
5′-
CTTGAGCTGCAGGGCAAAGA [symbol] [symbol]
CAGCGTGTATAGGGA [symbol] [symbol]
TATGTAGCATGGCCCGGGAA

20 bp spacer:

FP1:
(SEQ ID NO: 37)
5′-GGGATAGTGAAATTTATGAT
(Tm 54) (Taq 45)
RP1:
(SEQ ID NO: 38)
5′-GGGACCATCTACATATTATTATATT
(Tm 57) (Taq 48)

FP1 and RP1 produce 111 bp product.

### Final seq:

(SEQ ID NO: 39)
5′-
GGGATAGTGAAATTTATGAT [symbol] [symbol]
TGCTTTTCCCGGGTGCACAA [symbol] [symbol]
AATATAATAATATGTAGATGGTCCC

25 bp spacer:

FP1:
(SEQ ID NO: 26)
5’-CTTGAGCTGCAGGGCAAA (Tm 68)(Taq 58)
RP1:
(SEQ ID NO: 40)
5’- [symbol] [symbol] CTATACACGCTGCCTGGG
(Tm 64)(Taq 58)
FP2:
(SEQ ID NO: 41)
5’- [symbol] [symbol] AGGGACATAGGGAGATGGCTC
(Tm 68)(Taq 61)
RP2:
(SEQ ID NO: 29)
5’-TTCCCGGGCCATGCTACA (Tm 72)(Taq 62)

FP1 and RP1 generate 73bp product, FP2 and RP2 generate 74 bp product.

FP1 and RP2 with round 1 products as templates generate 111 bp product.

### Final seq:

(SEQ ID NO: 42)
5’-CTTGAGCTGCAGGGCAAAGA [symbol] [symbol] CAGCGTGTATAG
AGGGACATAGGGA [symbol] [symbol] TATGTAGCATGGCCCGGG
AA

30 bp spacer:

FP1:
(SEQ ID NO: 43)
5’-CTAGTAGCCCGGGGCATT (Tm 66)(Taq 60)
RP1:
(SEQ ID NO: 44)
5’-GGGCTCAATTATTCAGCATA (Tm 61)(Taq 51)

FP1 and RP1 produce 116 bp product.

### Final seq:

(SEQ ID NO: 45)
5’-CTAGTAGCCCGGGGCATTGT [symbol] [symbol] ATCTAATCATATC
CCGGGATGAAGGTCTAT [symbol] [symbol] TATGCTGAATAATTG
AGCCC

35 bp spacer:

FP1:
(SEQ ID NO: 26)
5’-CTTGAGCTGCAGGGCAAA (Tm 68)(Taq 58)
RP1:
(SEQ ID NO: 46)
5’- [symbol] [symbol] TTCACTATACACGCTGCCTG
(Tm 66)(Taq 57)
FP2:
(SEQ ID NO: 47)
5’- [symbol] [symbol] AGGAAGGGACATAGGGAGATGGC
(Tm 71)(Taq 63)
RP2:
(SEQ ID NO: 29)
5’-TTCCCGGGCCATGCTACA (Tm 72)(Taq 62)

FP1 and RP1 generate 78 bp product, FP2 and RP2 generate 73 bp product.

FP1 and RP2 with round 1 products as templates generate 121 bp product.

### Final seq:

(SEQ ID NO: 48)
5’-CTTGAGCTGCAGGGCAAAGA [symbol] [symbol] CAGCGTGTATAG
TGAAAAAGGAAGGGACATAGGGA [symbol] [symbol] TATGTAGC
ATGGCCCGGGAA

40 bp spacer:

FP1:
(SEQ ID NO: 49)
5’-GGCCCGGGAAGTACAGTAGA (Tm 67)(Taq 61)
RP1:
(SEQ ID NO: 50)
5’-ACAATGCCCCGGGCTACTAG (Tm 69)(Taq 62)

FP1 and RP1 produce 126 bp product.

### Final seq:

(SEQ ID NO: 51)
5’-GGCCCGGGAAGTACAGTAGA [symbol] [symbol] TGCTACTTACAT
ATTCTCCCGGGTAAATTAATTCTTATGA [symbol] [symbol] CTAG
TAGCCCGGGGCATTGT

50 bp spacer:

FP1:
(SEQ ID NO: 26)
5’-CTTGAGCTGCAGGGCAAA (Tm 68)(Taq 58)
RP1:
(SEQ ID NO: 29)
5’-TTCCCGGGCCATGCTACA (Tm 72)(Taq 62)

FP1 and RP1 produce 136 bp product.

### Final seq:

(SEQ ID NO: 52)
5’-CTTGAGCTGCAGGGCAAAGA [symbol] [symbol] CAGCGTGTATAG
TGAAAAGGAACCCGGGGATGGAGGAAGGGACATAGGGA [symbol] [symbol]
TATGTAGCATGGCCCGGGAA

### 2Block 2 with Inverted A and B Target Sites

(SEQ ID NO: 53)
GGGTTTGCTGCTCATCTATACTTTCACAATCTTGAGCTGCAGGGCAAAG
ACTACAATGGGATTAATAAATTGTACTCTAA
AGGATATTGAAAACTTGTGAGCTCCCTACGCATGCGTCCCAGGCAGCGT
GTATAGTGAAAAGGAACCCGGGGATGGAGGA
AGGGACATAGGGACCGCGCGACAAACCTGAGCCATCTATGTAGCATGGC
CCGGGAAGTACAGTAGAGCTCCCTACGCATG
CGTCCCAGGTGCTACTTACATATTCTCCCGGGTAAATTAATTCTTATGA
CCGCGCGACAAACCTGAGCCATCCTAGTAGC
CCGGGGCATTGTGCTCCCTACGCATGCGTCCCAGGATCTAATCATATCC
CGGGATGAAGGTCTATCCGCGCGACAAACCT
GAGCCATCTATGCTGAATAATTGAGCCCGGGATAGTGAAATTTATGATG
CTCCCTACGCATGCGTCCCAGGTGCTTTTCC
CGGGTGCACAACCGCGCGACAAACCTGAGCCATCAATATAATAATATGT
AGATGGTCCCGGGTAGGTTGTTATACATTTA
CTGAGCTCCCTACGCATGCGTCCCAGGTTTGTAGAAGGCTAGGGGAACA
GGTTAGTTTGAGGGAATTCTAATGGATCCTT
CTATGGG

PCR and OE-PCR on gBlock 2 to generate spacers from 6 bp to 50 bp. Bold indicates mispriming), [symbol] indicates Target Site A, [symbol] indicates Target Site B.

6 bp spacer:

FP1:
(SEQ ID NO: 54)
5’-ACTCTAAAGGATATTGAAAACTTGTGA (Tm 63)(Taq 53)
RP1:
(SEQ ID NO: 55)
5’- [symbol] [symbol] CTGCCTGGGACGCATG
(Tm 69)(Taq 60)
FP2:
(SEQ ID NO: 56)
5’- [symbol] [symbol] GGACCGCGCGACAAACCT
(Tm 73)(Taq 64)
RP2:
(SEQ ID NO: 57)
5’-ACTGTACTTCCCGGGCCA (Tm 68)(Taq 61)

FP1 and RP1 generate 70 bp product, FP2 and RP2 generate 70 bp product.

FP1 and RP2 with round 1 products as templates generate 106 bp product.

### Final seq:

(SEQ ID NO: 58)
5’-ACTCTAAAGGATATTGAAAACTTGTGA [symbol] [symbol] CAGGG
A [symbol] [symbol] TATGTAGCATGGCCCGGGAAGTACAGT

10 bp spacer:

FP1:
(SEQ ID NO: 54)
5’-ACTCTAAAGGATATTGAAAACTTGTGA (Tm 63)(Taq 53)
RP1:
(SEQ ID NO: 59)
5’- [symbol] [symbol] CGCTGCCTGGGACGCAT
(Tm 74)(Taq 65)
FP2:
(SEQ ID NO: 60)
5’- [symbol] [symbol] AGGGACCGCGCGACAAAC
(Tm 73)(Taq 64)
RP2:
(SEQ ID NO: 57)
5’-ACTGTACTTCCCGGGCCA (Tm 68)(Taq 61)

FP1 and RP1 generate 73 bp product, FP2 and RP2 generate 72 bp product.

FP1 and RP2 with round 1 products as templates generate 110 bp product.

### Final seq:

(SEQ ID NO: 61)
5’-ACTCTAAAGGATATTGAAAACTTGTGA [symbol] [symbol] CAGCG
AGGGA [symbol] [symbol] TATGTAGCATGGCCCGGGAAGTACAG
T

15 bp spacer:

FP1:
(SEQ ID NO: 54)
5’-ACTCTAAAGGATATTGAAAACTTGTGA
(Tm 63)(Taq 53)
RP1:
(SEQ ID NO: 62)
5’- [symbol] [symbol] ATACACGCTGCCTGGGAC
(Tm 66)(Taq 60)
FP2:
(SEQ ID NO: 63)
5’- [symbol] [symbol] AGGGACCGCGCGACAAAC
(Tm 73)(Taq 64)
RP2:
(SEQ ID NO: 57)
5’-ACTGTACTTCCCGGGCCA (Tm 68)(Taq 61)

FP1 and RP1 generate 78 bp product, FP2 and RP2 generate 73 bp product.

FP1 and RP2 with round 1 products as templates generate 115 bp product.

### Final seq:

(SEQ ID NO: 64)
5’-ACTCTAAAGGATATTGAAAACTTGTGA [symbol] [symbol] CAGCG
TGTATAGGGA [symbol] [symbol] TATGTAGCATGGCCCGGGAAG
TACAGT

20 bp spacer:

FP1:
(SEQ ID NO: 37)
5’-GGGATAGTGAAATTTATGAT (Tm 54)(Taq 45)
RP1:
(SEQ ID NO: 38)
5’-GGGACCATCTACATATTATTATATT (Tm 57)(Taq 48)

FP1 and RP1 produce 111 bp product.

### Final seq:

(SEQ ID NO: 65)
5’-GGGATAGTGAAATTTATGAT [symbol] [symbol] TGCTTTTCCCGGG
TGCACAA [symbol] [symbol] AATATAATAATATGTAGATGGTCC
C

25 bp spacer:

FP1:
(SEQ ID NO: 54)
5’-ACTCTAAAGGATATTGAAAACTTGTGA (Tm 63)(Taq 53)
RP1:
(SEQ ID NO: 66)
5’- [symbol] [symbol] CTATACACGCTGCCTGG
(Tm 60)(Taq 55)
FP2:
(SEQ ID NO: 67)
5’- [symbol] [symbol] AGGGACATAGGGACCGC
(Tm 65)(Taq 59)
RP2:
(SEQ ID NO: 57)
5’-ACTGTACTTCCCGGGCCA (Tm 68)(Taq 61)

FP1 and RP1 generate 79 bp product, FP2 and RP2 generate 80 bp product.

FP1 and RP2 with round 1 products as templates generate 125 bp product.

### Final seq:

(SEQ ID NO: 68)
5’-ACTCTAAAGGATATTGAAAACTTGTGA [symbol] [symbol] CAGCG
TGTATAGAGGGACATAGGGA [symbol] [symbol] TATGTAGCATG
GCCCGGGAAGTACAGT

30 bp spacer:

FP1:
(SEQ ID NO: 43)
5’-CTAGTAGCCCGGGGCATT (Tm 66)(Taq 60)
RP1:
(SEQ ID NO: 44)
5’-GGGCTCAATTATTCAGCATA (Tm 61)(Taq 51)

FP1 and RP1 produce 116 bp product.

### Final seq:

(SEQ ID NO: 69)
5’-CTAGTAGCCCGGGGCATTGT [symbol] [symbol] ATCTAATCATATC
CCGGGATGAAGGTCTAT [symbol] [symbol] TATGCTGAATAATT
GAGCCC

35 bp spacer:

FP1:
(SEQ ID NO: 54)
5’-ACTCTAAAGGATATTGAAAACTTGTGA (Tm 63)(Taq 53)
RP1:
(SEQ ID NO: 70)
5’- [symbol] [symbol] TTTTTCACTATACACGCTGCCT
(Tm 63)(Taq 56)
FP2:
(SEQ ID NO: 71)
5’- [symbol] [symbol] AGGAAGGGACATAGGGACC
(Tm 64)(Taq 58)
RP2:
(SEQ ID NO: 57)
5’-ACTGTACTTCCCGGGCCA (Tm 68)(Taq 61)

FP1 and RP1 generate 87 bp product, FP2 and RP2 generate 88 bp product.

FP1 and RP2 with round 1 products as templates generate 135 bp product.

### Final seq:

(SEQ ID NO: 72)
5’-ACTCTAAAGGATATTGAAAACTTGTGA [symbol] [symbol] CAGCG
TGTATAGTGAAAAAGGAAGGGACATAGGGA [symbol] [symbol] T
ATGTAGCATGGCCCGGGAAGTACAGT

40 bp spacer:

FP1:
(SEQ ID NO: 49)
5’-GGCCCGGGAAGTACAGTAGA (Tm 67)(Taq 61)
RP1:
(SEQ ID NO: 50)
5’-ACAATGCCCCGGGCTACTAG (Tm 69)(Taq 62)

FP1 and RP1 produce 126 bp product.

### Final seq:

(SEQ ID NO: 73)
5’-GGCCCGGGAAGTACAGTAGA [symbol] [symbol] TGCTACTTACAT
ATTCTCCCGGGTAAATTAATTCTTATGA [symbol] [symbol] CTAG
TAGCCCGGGGCATTGT

50 bp spacer:

FP1:
(SEQ ID NO: 54)
5’-ACTCTAAAGGATATTGAAAACTTGTGA (Tm 63)(Taq 53)
RP1:
(SEQ ID NO: 57)
5’-ACTGTACTTCCCGGGCCA (Tm 68)(Taq 61)

FP1 and RP1 produce 150 bp product.

### Final seq:

(SEQ ID NO: 74)
5’-ACTCTAAAGGATATTGAAAACTTGTGA [symbol] [symbol] CAGCG
TGTATAGTGAAAAGGAACCCGGGGATGGAGGAAGGGACATAGGGA [symbol] [symbol]
GCCCGGGAAGTACAGT

Everted A & B Sites—Target B Rev followed by Target A Fwd

PCR and OE-PCR on gBlock 2 to generate spacers from 6 bp to 50 bp. Bold indicates mispriming), [symbol] indicates Target Site A, [symbol] indicates Target Site B

6 bp spacer:

FP1:
(SEQ ID NO: 75)
5’-CCGGGTAAATTAATTCTTATGA (Tm 60)(Taq 49)
RP1:
(SEQ ID NO: 76)
5’- [symbol] [symbol] TAGGATGGCTCAGGTTTG
(Tm 61)(Taq 53)
FP2:
(SEQ ID NO: 77)
5’- [symbol] [symbol] TGTGCTCCCTACGCATGC
(Tm 69)(Taq 61)
RP2:
(SEQ ID NO: 78)
5’-ATCTAATCATATCCCGGGATGA (Tm 65)(Taq 54)

FP1 and RP1 generate 66 bp product, FP2 and RP2 generate 66 bp product.

FP1 and RP2 with round 1 products as templates generate 96 bp product.

### Final seq:

(SEQ ID NO: 79)
5′-CCGGGTAAATTAATTCTTATGA [symbol] [symbol] CTATGT [symbol] [symbol] [symbol]
ATCTAATCATATCCCGGGATGA

10 bp spacer:

FP1:
(SEQ ID NO: 75)
5′-CCGGGTAAATTAATTCTTATGA (Tm 60)(Taq 49)
RP1:
(SEQ ID NO: 80)
5′- [symbol] [symbol] ACTAGGATGGCTCAGGTT
(Tm 59)(Taq 54)
FP2:
(SEQ ID NO: 81)
5′- [symbol] [symbol] ATTGTGCTCCCTACGCAT
(Tm 63) (Taq 56)
RP2:
(SEQ ID NO: 78)
5′-ATCTAATCATATCCCGGGATGA (Tm 65)(Taq 54)

FP1 and RP1 generate 68 bp product, FP2 and RP2 generate 68 bp product.

FP1 and RP2 with round 1 products as templates generate 100 bp product.

### Final seq:

(SEQ ID NO: 82)
5′-CCGGGTAAATTAATTCCTTATGA [symbol] [symbol] CTAGTATTGT [symbol] [symbol]
ATCTAATCATATCCCGGGATGA

15 bp spacer:

FP1:
(SEQ ID NO: 75)
5′-CCGGGTAAATTAATTCTTATGA (Tm 60)(Taq 49)
RP1:
(SEQ ID NO: 83)
5′- [symbol] [symbol] CTACTAGGATGGCTCAGG
(Tm 57)(Taq 53)
FP2:
(SEQ ID NO: 84)
5′- [symbol] [symbol] GGCATTGTGCTCCCTACG
(Tm 67)(Taq 59)
RP2:
(SEQ ID NO: 78)
5′-ATCTAATCATATCCCGGGATGA (Tm 65)(Taq 54)

FP1 and RP1 generate 70 bp product, FP2 and RP2 generate 71 bp product.

FP1 and RP2 with round 1 products as templates generate 105 bp product.

### Final seq:

(SEQ ID NO: 85)
5′-CCGGGTAAATTAATTCTTATGA [symbol] [symbol] CTAGTAGGGC
ATTGT [symbol] [symbol] ATCTAATCATATCCCGGGATGA

20 bp spacer:

FP1:
(SEQ ID NO: 75)
5′-CCGGGTAAATTAATTCTTATGA (Tm 60)(Taq 49)
RP1:
(SEQ ID NO: 78)
5′-ATCTAATCATATCCCGGGATGA (Tm 65)(Taq 54)

FP1 and RP1 produce 110 bp product.

### Final seq:

(SEQ ID NO: 86)
5′-CCGGGTAAATTAATTCTTATGA [symbol] [symbol] CTAGTAGCCC
GGGGCATTGT [symbol] [symbol] ATCTAATCATATCCCGGGATGA

25 bp spacer:

FP1:
(SEQ ID NO: 87)
5′-TTCTCCCGGGTAAATTAATTCTTATGA (Tm 67)(Taq 55)
RP1:
(SEQ ID NO: 88)
5′- [symbol] [symbol] CCCGGGCTACTAGGATGG
(Tm 67)(Taq 60)
FP2:
(SEQ ID NO: 89)
5′- [symbol] [symbol] CCGGGGCATTGTGCTCCC
(Tm 76)(Taq 66)
RP2:
(SEQ ID NO: 90)
5′-GACCTTCATCCCGGGATATGATTAGAT (Tm 71)(Taq 60)

FP1 and RP1 generate 81 bp product, FP2 and RP2 generate 80 bp product.

FP1 and RP2 with round 1 products as templates generate 125 bp product.

### Final seq:

(SEQ ID NO: 91)
5′-TTCTCCCGGGTAAATTAATTCTTATGA [symbol] [symbol] CTAGT
AGCCCGGGCCGGGGCATTGT [symbol] [symbol] ATCTAATCATATC
CCGGGATGAAGGTC

30 bp spacer:

FP1:
(SEQ ID NO: 92)
5′-GATGGAGGAAGGGACATAGG (Tm 65)(Taq 57)
RP1:
(SEQ ID NO: 93)
5′-CCGGGAGAATATGTAAGTAGCA (Tm 64)(Taq 56)

FP1 and RP1 produce 120 bp product.

### Final seq:

(SEQ ID NO: 94)
5′-GATGGAGGAAGGGACATAGGGA [symbol] [symbol] TATGTAGCA
TGGCCCGGGAAGTACAGTAGA [symbol] [symbol] TGCTACTTACA
TATTCTCCCGG

35 bp spacer:

FP1:
(SEQ ID NO: 92)
5′-GATGGAGGAAGGGACATAGG (Tm 65)(Taq 57)
RP1:
(SEQ ID NO: 95)
5′- [symbol] [symbol] CCGGGCCATGCTACATA
GAT (Tm 68)(Taq 59)
FP2:
(SEQ ID NO: 96)
5′- [symbol] [symbol] CCCGGGAAGTACAGTAGA
GCT (Tm 66)(Taq 61)
RP2:
(SEQ ID NO: 93)
5′-CCGGGAGAATATGTAAGTAGCA (Tm 64)(Taq 56)

FP1 and RP1 generate 83 bp product, FP2 and RP2 generate 83 bp product.

FP1 and RP2 with round 1 products as templates generate 125 bp product.

### Final seq:

(SEQ ID NO: 97)
5′-GATGGAGGAAGGGACATAGGGA [symbol] [symbol] TATGTAGCA
TGGCCCGGCCCGGGAAGTACAGTAGA [symbol] [symbol] TGCTAC
TTACATATTCTCCCGG

40 bp spacer:

FP1:
(SEQ ID NO: 98)
5′-ATCCCGGGATGAAGGTCTAT (Tm 66)(Taq 57)
RP1:
(SEQ ID NO: 99)
5′-TTGTGCACCCGGGAAAA (Tm 69)(Taq 57)

FP1 and RP1 produce 126 bp product.

### Final seq:

(SEQ ID NO: 100)
5′-ATCCCGGGATGAAGGTCTAT [symbol] [symbol] TATGCTGAATA
ATTGAGCCCGGGATAGTGAAATTTATGAT [symbol] [symbol] TGCT
TTTCCCGGGTGCACAA

50 bp spacer:

FP1:
(SEQ ID NO: 101)
5′-TTTTCCCGGGTGCACAAC (Tm 69)(Taq 59)
RP1:
(SEQ ID NO: 102)
5′-GTTCCCCTAGCCTTCTACAAACC (Tm 67)(Taq 60)

FP1 and RP1 produce 134 bp product

### Final seq:

(SEQ ID NO: 103)
5′-TTTTCCCGGGTGCACAA [symbol] AATATAATAATATGT
AGATGGTCCCGGGTAGGTTGTTATA
CATTTACTGA [symbol] [symbol] TTTGTAGAAGGCTAGGGGAAC

### Supplementary Methods 4: Western Blot for Verification of Purified Fusion Proteins for RNPs

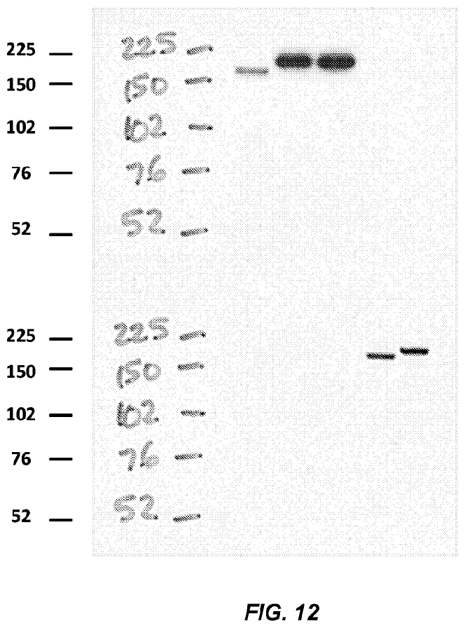

See FIG. 12, which shows a western Blot for HA epitope tagged proteins (top, left to right: SmBiT-dCas9, LgBiT-dCas9, NLuc-dCas9) and a western Blot for 3X-Flag epitope tagged proteins (bottom, left to right: dCas9-SmBiT, dCas9-LgBiT).

### Supplementary Methods 5: Process for Creation of gRNAs for MUC4 DNA Biosensing

Repetitive region in exon 2:
MUC4 repetitive DNA region 48 bp repeat:

(SEQ ID NO: 104)
5′-GCCACCCCTCTTCCTGTCACCGACACTTCCTCAGCATCCAC [symbol] TCAC~GCC-3′
(SEQ ID NO: 105)
3′-C [symbol] AGAAGGACAGTGGCTGTGAA [symbol] [symbol] -5′
sgMUC4-E3(F + E):
(SEQ ID NO: 106)
GGCGTGACCTGTGGATGCTG [symbol]
MUC4 gRNA tgt 1:
(SEQ ID NO: 107)
GACACTTCCTCAGCATCCAC

[symbol]

Everted overlapping, PAMs 10 bp apart

### CFD:110.89

MUC4 gRNA tgt 2:
(SEQ ID NO: 108)
GGTGGATGCTGAGGAAGTGTCGG

Tandem overlapping, PAMs 6 bp apart

### CFD:163.22

MUC4 gRNA tgt 3:
(SEQ ID NO: 109)
GGTGAGGAAGTGTCGGTGACAGG

Tandem overlapping, PAMs 13 bp apart

### CFD:70.68

MUC4 gRNA tet 4:
(SEQ ID NO: 110)
GAAGTGTCGGTGACAGGAAGA

[symbol]

Tandem overlapping by 1 bp, PAMs 19 bp apart

### CFD:118.16

MUC4 gRNA tgt 5:
(SEQ ID NO: 111)
GGTGTCGGTGACAGGAAGAG [symbol] G

### Tandem 1 bp

### CFD:122.54

MUC4 gRNA tgt 6:
(SEQ ID NO: 112)
GGCGGTGACAGGAAGAGGGG

[symbol]

### Tandem 4 bp

### CFD:227.72

Selected gRNAs 1-4 for Experiments
Non-repetitive region in intron 1:
MUC4 non-repetitive DNA region with Cas9 target sites:

(SEQ ID NO: 113)
ATGAAGGGGGCACGCTGGAGGAGGGTCCCCTGGGTGTCCCTGAGCTG
CCTGTGTCTCTGCCTCCTTCCGCATGTGGTCCCAGGTAAGTGATGGA
GACAGCAGATGAGGCTGGCTGCGGGGAGCACTTGGGGGAGGTGGGAG
CTGTCAGAGAAAGAGGTCCGGGGAGACAGAGAGAGAGAGAGAGAGAA
TAGGGGAAAGGGAGACAGCGAAGAGGAAGAGAAGGGAGAGAAAAAGA
GGGAGAGGGAAAGGAGAAAGAGATGAATGGGACAACATGGGGGGAAG
GTGGAGAGAGACCCAGAGAGGGAAAGAAGAGGAAGAGAAGAGGGAGA
GAGAAAGAAGAGTGGAGGCCGTGCGCGGTGGCTCATGCCTGTAATCC
CAGCACTTTCGGAGGCCAAGGCAGGAGATCACCTGAGGTCAGGAGTT
CGAGACCAGCCTGGCCGACATGGTGAAACCCCGTCTCTACTAAATAT
ACAAAAATTAGCCGGTCGTGGTGGGCCCCACCTGTAATTCCAGCTAC
TCAGGAGTCTGAGGCAGGAGAATCACTTGAACCTGGGAGGTGGAGGT
TGCAGTGAGCCAAGATCGCGCCACTGCACTCCAGCCTGGGAGAGAGA
GCGAGACTCTGTCTCAAAATAAATAAATAAATAAATAAATAAATAAA
TAAATAAATAAATATAAAATAAAATAAAAAATAAGAAGAGGAGAAAA
GTGGGGAAGAGGAGGCATGAACTGGCAGATACGGGACAAGATCTGAG
GGGAAAGACAGAGGGAGAATGCTCGAAAGAGAGAGAAAAGAGAACAG
AGGGCCAGAGAGCAGCCCGGCGATGTCTGGAAGGATCCATGGTGAGA
GCCCAGGCTTACTCGCAGAGAGAAAGACAGGCAGAGCCAGAGCAAGA
GGAACAGAGTCAAGGAGAAAGATGTACACCCTTGTGTACAGAGCTGG
GGGTAGAGGGGATGCCAGGAAAGCTGGGTGATGGAGACGGAAGAAAA
CTCATGTAAAGCTGCAGGGTGAGAGGACGAGACAGGTGAGACGCAGA
CAAACTGAGGACCCTGGGAATGGAGAGAGGAGAAGATCGGGAGACAG
CAGCAAGCAAGGGAAGCGACAAGGAGGAGAGGGGCAGGCCGGCCGGG
AGGGTGGTGCGGAGGAGGCGGCCAGGGCGCAGAGGGCCGGGAGGTGC
TGGCCGTGGGCTTCTTACCTCTGAGCTCGGGTTTAAAAGCCTCCATT
TGGGTCACGGCCTTGCCTGGGGCTCGTAGCCCCGGCATTGGCCTTGG
GCTCCTCCGTGTACAGAGCTGGGAGGGGAGGGATGCCAGGCCTGTGG
GAGATGTTCCCTCGGGGGCCCCCGTCCTCTTCCCCACACTTTCCAGG
CTGTCCCTCTGGCTTCAGGACCAAGTTTTATTCTGTGTTTCTGGGTG
TCTGAGTCTTTGGGGGAGAGTCTGGGGTCCAGAGTTCAAGCTGGGGT
TAGAGTCTCAGCTCCTGCCCTGCCTCTCAGCAGGCTAAGAACAGTCG
CCGAGGGAAAATATTTCTTGGGCGCATATTTGAGGAGCTTCCTGGGA
GTGAGTCAGAAGGCGAGTGCCGTTTAAAGGCTGCAAGAGAAGCCATG
CTGGTGAAGCGGACCCTTCCACCTCGGGATGTTTCAGGACTAGGCTG
AGGGCAAAGGAAACTGCCACCACCTCCCTACACCTCCCCACCCTCCA
GCACCCCCACCCCACCCTGGCCACACAACCCCGCTCCAGTGCTCATC
CCACCGTGAGGACGTGGAGGCCGGAAGGAGCCGCCACACGGCCCTGC
CCTGCAGATGTGGTTGAAGGAGTCTCCACGGGAATCATGACTCCCAG
AGCGAGGCTGGGGCTTGGGGCGCCGGGGAGGCAGCTTGGATTTAGGA
GCCCCAGGGCCAAGTCTTTGCCGTGAACTGTTCTGGCCCCTGTGACC
AGGCCCTGCCCCGTGTCTCCCCAGGGCCCCGGTCCCCTGTGTAAAAA
GCAGTGGTGAACGGTTGGACCTCCTGACGCCCAAGTTCTTGAGTTTC
CAAATCTGTGATTTAAAGCTGAGCCCAAATGTGCTGGGTACCAGCTG
GACACTCAGCTCCATGTGGAGCCAGGAAGTGGGGTCTGTGGAGAGGA
GCGCAGAGGGGCAAGACCTGGGGTGGGCGTGGAAAAGCACGGGGGCG
TGACCCGGAGAAGGAGTGAAGGACTGTTGGTGTGCAAGGGCGTCTCC
ATGACGACCCGAAGAAGCTAGGCATGTCGTGGAGCGCTGAGTCCTTT
GCGTCGCTAAGGGGACCAAGTGGAGCTGGGCCAGGAGAGGAGATGGT
CGTGGCTGGGAGATGGCACCCACACATCTGACCGGGCATGACCAGGG
CCTTGGCAGGAAAAGCAGTCACCAAGGGCGGGTGGGCAGCCCCCACC
CCCACAGGGCAGCTGCTGGAGGACTGGCAGCCAGCCAGCCCCGTTCC
TTTTGGCTCCCTGAAGGGGTTTACAGATGACCTGCCTATACTTGAGT
CTAGGGTCTGTTTGCACACTTGCCGGCAGGACCCTCACCCAGGCTGG
GTCACACTGAAGCCCAGGCCAGAGGAAAAACACAGGGTTTCCACAAA
GGAGCTGCCGCAATGAGGGTTTCCTTAAGGAACAGCCCTGGCTCTCA
AGGGTTAAAGGATAAGGCACAGCAGACAGAGGTGGGCTAGACAAGGA
CAGATGGAAATTTGGTGTCTACTGGTCGCCCCAGGCAGGAATGACTC
AGAAGGAAGCCTGGCCGTCCTGGTTCCATGCCACAGGGAAAGGCAAC
TGGGTCGAAATAGGCCTTGGTCTCCAGCACTATCAGTGACCCCAGGG
AGGTGACAGGCTGGAGCAAGTGCAGGGCAGGCAGGGGAGGGGACGCC
GGCCACAGCGCACTCCACGGGGAAGGGTCTTTATGGGCCCCTCCTCG
GAGAACCCCCGGTCTATCTGTCAGTCTGGGACAGGCCACCTCAACTT
GCCACCGAGGACACCAAAACTCTCCACAGACCCCTCTGCCCCTCTGG
GAAACCCCACTGTGCTCCAGGACACTCAAAAGGAAAGGATCCCTGGA
CAAGAGGTCCTGCCAGGAACATCAGCCAAATTTTGGCCAACGACCAG
CAAGGTGCACAGGGAAGAGCAGGGGCTGAAACTCAGAGGTCCAGCAT
CAGCGACGCCCTTGGCAGCCCAGGGAACACAGGCAACGCCTTTTGGC
TCTGGAGTCTTAGGCTCTTCATCGGCAAACTGAGCCCAGGGGGAAGG
GGCTACTACGTAGGGTTGTCATGAGGATGAAACGAGACAGCATCTGG
TGTAAAGTAGAAAAGGCATAAAGGGCCGGGCGCGGTGGCTCACGCTG
TAATCCCAGCACTTTTGGAGGCCCAGGCGGGTGGATCACCTGAGGTC
AGGAGTTCAAGACCAGCTTGGCCAACCCTGTCTCCACTAAAAATAAA
AAATTTTGCCGGGCGTGGTGGCGAGCGCCTGTAATTCCAGCTACTCG
GGAGGCTGAGGTAGGAGAATGGCTTGAACCTGGGAGGCAGAGGTTGC
AGGGAGCCGAAATGGCAGCACTCTAGCTTGGGTGACAGAGCAAGACT
CTGTCTAAAAAAAAAAGAAAAGCCATAAAGACGTGTTTGAGAAAGAG
GCCTGGGAAGACGGGGGAAGGAGGGTGATTGAACCCGGAATGGCACT
TGTGTCGGCCCAGGGTCATATCCCTTCATCTAAGGATCCTCGTGCCT
CTAAAAAGCCACCCCGTGCTTCCTGTGGGTTTGCAAGGGCTGGCTTG
GTGTATTCAGAATGTGGCTTGCTGCATGAACGGACCCCGAGGGCCAT
GGCCCTAGAGCAGGGGCTCGCTCCAGCGGACAGCTCTGCCTCACCGC
TCCCTGCCTGTGAGTCCCGCCACGCCCTTGGTTTCTGGGCTCAGCCG
TGGAGGCAGAGGCTGGCCTGGCAGAGGCTGGCCTGGCAGTGCTTGAC
ACGCAAGTGATTTGTGTCTTCATTGCTAAGGACAAGAGGCAATGAGA
GGACAAGAAGTGGTTGGCCTTTTGTACGCTCAACGGGTGGTTTTGCT
ACTCTGTGTCTTTTCTCTGATTTCACGGTGCTGTTAAGTGCTTAAAA
TATGCACATCGTGTAGCTCACAGAGCCACTTCTCTGAAGGCCAGGAC
AGAGACCTTATAGGCTCTCTCTCCCCCTAGTTTCAGCCTTTTACCTT
AAATATACGTCTTTCTTACTGCTAGGCTGAGTTCCCGCCCCAGCATG
TTCTGAGAAATTGAGTCAAAATAACTGAGTCTGTTGGCACCTCATCG
ACGATTTCTTCATAGACGGTTTTTTTATTGTTGCTGTTGTTGTTGGT
TTTTTGGGTTTGTTTGTTTGTTTTTTGAGACAGAGTTTCTCTCTGTC
CCCCAGGCTGCAGTGCAGTGGCGTGGTCTCAGCTCAGTGCAGCCTCT
GCCTCCCGGGTTCAAGAGATTCTCCTGCCTCAGCCTCCCGAGTAGCT
GGGATTATAGACGCCCAACACCACAGCGGCTAATGTTTGTATTTTTA
GTAGAGATGGGGTTTCACCATGTTGGCCAGGCTGGTCTCGAACTCCT
GACCTCAGGTGATCCGCTCGCCTCGGCTCCCAAAGTGCTGGGATTAT
AGGCGTGAGCTACTGTGCCTGGCCCTACTTCATAGAGGTTTAAATGC
CTTTTCACCCTTTTCCTGGAGACTCTGAAGAAGTCTCAGGAACTGGG
CATTTGTGTTGCACGTGAGGCCTTGCAATGGCGGCCCTGCTTGGAGG
AAGGGCACTGGCCTGGGTTGCCCGCAGCTCCACTCCCCGTGTATGTG
TTTAGGGACCACAGAGGACAGACATCGACTCTCTGTAGAGATGCCGC
CCCGCCCAGGTTGCAGTTTAGGTTCCAAAAGTCCAGTGGCCAGTGGA
TTTTGGGGGAATTTGGAATAAGAAACAGCCTAGACTTTGGAGTTGTT
CATTCACTTGCAGAATTTCTACTCATGCCAGCTGCTCTGGACAGGAA
GATGAATGCGTCACAGTTCCTGCTTTTCAAAGCTCTCTAAGTTAAGT
GACTTGTTTAAGATCATAGAACCCATAAGTGAGGCAGCTGGGACTAG
AACCCAGGTCTCCTGACTCACTGCAGCACACAGCCTTTCGGCAATCT
CCAAACCAGCCCAGCCCACCGACGGAGGGAAGAACAGAAGCATTCAC
ACACCCTGCTGAGACAGCCATTCATTCATTCATTTGTTAATTAAACC
ACCATTTAGGAAACGCCTGCCTTAAGTTCCTGACATTGTTCTAGGAC
ACAGCACTGGATGCACACAGTGAAGAGTGAAACAGACGTGGCCCAGT
CTCTTGGCACTAAAATCTTGGTGCAGACAGACATCAAATAATTACGG
AAATGTTCTCAACTGCACATGTGGTAAATGCAGTGTGGAAAAGTACA
GGGTGTGCTGAGAGCTGCATTTCGAATGGCCAGAGAGTAGGGGAGGT
GCATCTGACTGACAAGTCAGGAAGGGCCCTGTGAGGAACCGTTCTGC
GGGGAGCTGAGGCCTGAGGCTGAGGACAGCCAGGTGGAGAAGGTGCC
AGGCCTGAGCAGGCAGAGGCGGAGCTCATGGAGAGGCAGGAAAGAGC
TTGGCCCCTTGGAGGACTTGAAAGAGAAGGCAGG

gRNAs from low to high CFD
gRNA1: 1.62 w/tandem 10 bp nearby site; tandem overlapping PAMs w/gRNA4, everted 7 bp with gRNA7
gRNA2: 1.79 w/tandem overlapping, PAMs 17 bp apart nearby site
gRNA3: 2.94 w/tandem overlapping, PAMs 15 bp apart nearby site
gRNA4: 3.20 w/tandem 9 bp nearby site; tandem overlapping PAMs w/gRNA1, everted 8 bp w/gRNA7
gRNA5: 3.50 w/tandem overlapping, PAMs 4 bp apart nearby site
gRNA6: 4.13 w/everted overlapping, PAMs 15 bp apart nearby site
gRNA7: 4.82; everted 9 bp with gRNA1, everted 8 bp w/gRNA4
gRNA8: 5.26 w/tandem 12 bp nearby site
gRNA9: 6.29 w/tandem overlapping, PAMs 8 bp apart nearby site
gRNA10: 6.55 w/everted PAMs overlapping nearby site
gRNA11: 6.83 w/tandem overlapping, PAMs 7 bp apart nearby site; everted overlapping, PAMs 8 bp apart w/gRNA10
gRNA12: 7.25 w/tandem 3 bp nearby site
gRNAs 1-3, 5, 9-10, and 12 from this list selected to bind loci 1-7 in FIG. 10.

### Supplementary Methods 6: Process for Creation of gRNAs for 8q24 and PALB2 Editing and Edit Biosensing

8q24 risk locus (+) chr8:127,400,950-127,401,200
(SEQ ID NO: 114)
AAGAAAAAAATAATTAGAATGTCTTGTTTTTTAAATGACTCTTCATT
TTGTTGTTAATATCTGGTTTTAATTAGAAATTGCGGTTACAATATGG
AAGACTTGAATTTAAAGGTAAAGCTCTTGGATAACAGGTTTAAGGGA
AGGAAACTT [symbol] TTTCTTAGAGCACTAGGTAGAATTCTAATGATAAA
AGAGGCTCTCACGCACAAAGACTGGATTCTCTCTTAGTTTTGAAAAT
GTTCAGACACAGAAAT
(SEQ ID NO: 115)
tctcagctccctatccataaaacagagggacgaataa [symbol]
tgtag

[symbol]

[symbol]

[symbol]

[symbol]

[symbol]

CACTGAGAAAAGTACAAAGAATTTTTATGTGCTATTGACTT [symbol] [symbol]
GAGCCGGCCCCAGCTGGAAAGC
TGCTTTCTCTGAATCAAAGGGCAGGAACCCAGCAAGTTTCTCAGGATT
GGGGCC

### Used in Editing

g259 (G->T edit):
(SEQ ID NO: 116)
CTTTGAGCTCAGCAGATGAAAGG
g248 (inverted overlapping):
(SEQ ID NO: 117)
CTGAGCTCAAAGGACGATGA

[symbol]

New designs

8q24gRNA1 (inverted 0 bp):
(SEQ ID NO: 118)

[symbol]

[symbol]

CFD 5.24
8q24gRNA2 (tandem 28 bp):
(SEQ ID NO: 119)

[symbol]

8q24gRNA3 (tandem 41 bp):
(SEQ ID NO: 120)

[symbol]

[symbol]

### Palb2 Locus (+) Chr16:23,624,025-23,624,175

(SEQ ID NO: 121) [symbol] [symbol]
ACGAGATTATACACATCAGGCACTGG
AACTATCTGTAATACTGGAACCTAAATAAAACAAAGCAG [symbol] [symbol] [symbol]
CATTTTTGTTTAATCCAGATTTTCCAAAATTTATCA
CATT

### Used in Editing

gPalbMis1 (C->A missense):
(SEQ ID NO: 122)
ACTGGAACTATCTGTAATACTGG
gPalbMis2 (tandem 15 bp):
(SEQ ID NO: 123)

[symbol]

[symbol]

New designs

Palb2gRNA1 (tandem overlapping, PAMs 15 bp apart):
(SEQ ID NO: 124) [symbol] [symbol]
CFD 23.05
Palb2gRNA2 (everted 21 bp):
(SEQ ID NO: 125) [symbol] [symbol]
CFD 28.08
Palb2gRNA3 (inverted 21 bp):
(SEQ ID NO: 126)

[symbol]

[symbol]

Palb2gRNA4 (tandem 21 bp):
(SEQ ID NO: 127)
CACACGAGATTATACACATCAGG

### Example 3. Sensing Repetitive and Nonrepetitive Regions of MUC4 in Individual Cells of Six Cell Lines

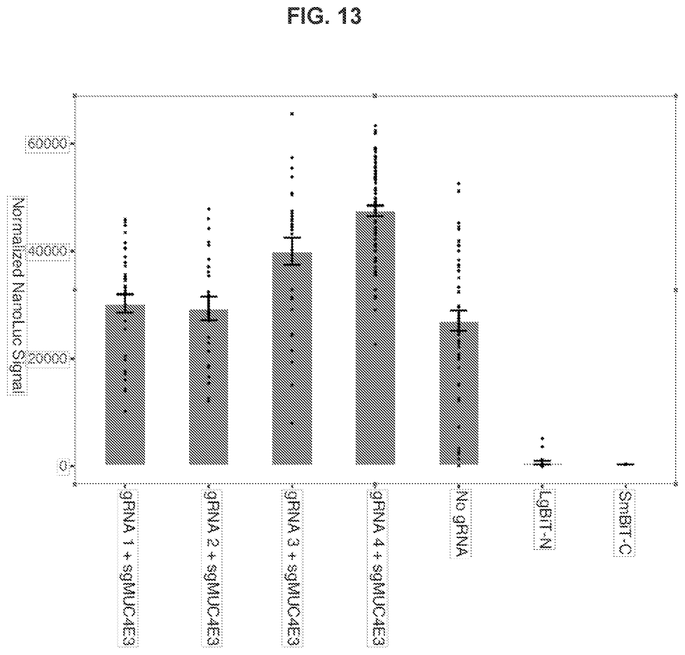

The data from FIG. 4 demonstrated the ability to detect repetitive and nonrepetitive regions of the MUC 4 locus in bulk groups of cells. However, greater utility would be to detect these sequences in individual cells since that could enable, for example, individually edited cells to be identified and isolated for clonal expansion. We therefore repeated the experiment transfecting plasmid DNA encoding the split-probes, a GFP transfection reporter, and sgMUC4-E3 and additional sg1-sg4 targeting the 100-400 repeats of MUC4 in HEK 293T cells. Although there was considerable variation in the luminescence of individual cells, we detected the repetitive target region with a peak of approximately 2-fold signal-to-noise (sg4 compared to no gRNA) (FIG. 13). This was similar to our previous result in groups of cells (FIG. 4H), suggesting that observations in groups of cells were predictive of those in individual cells.

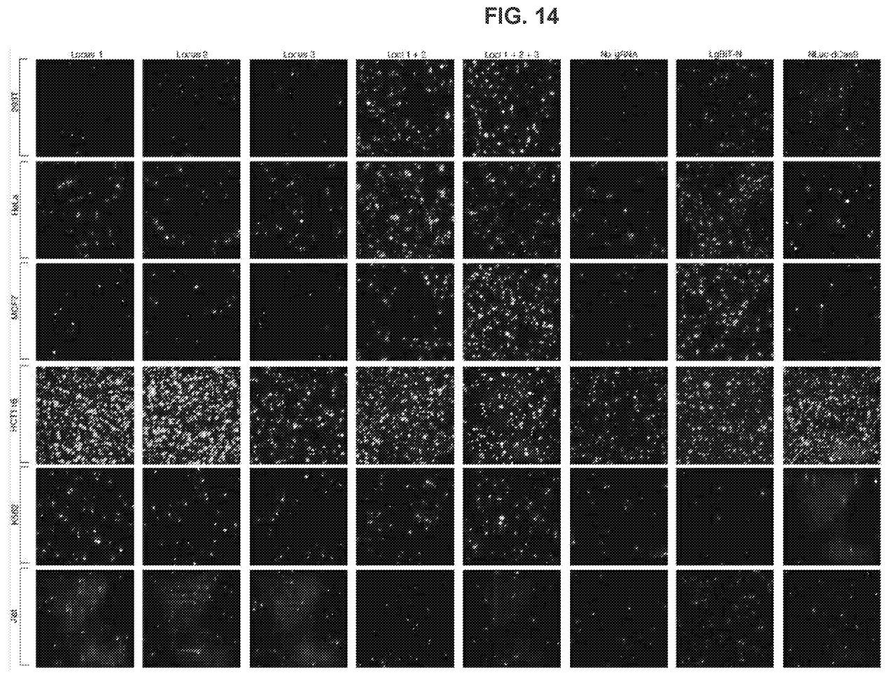

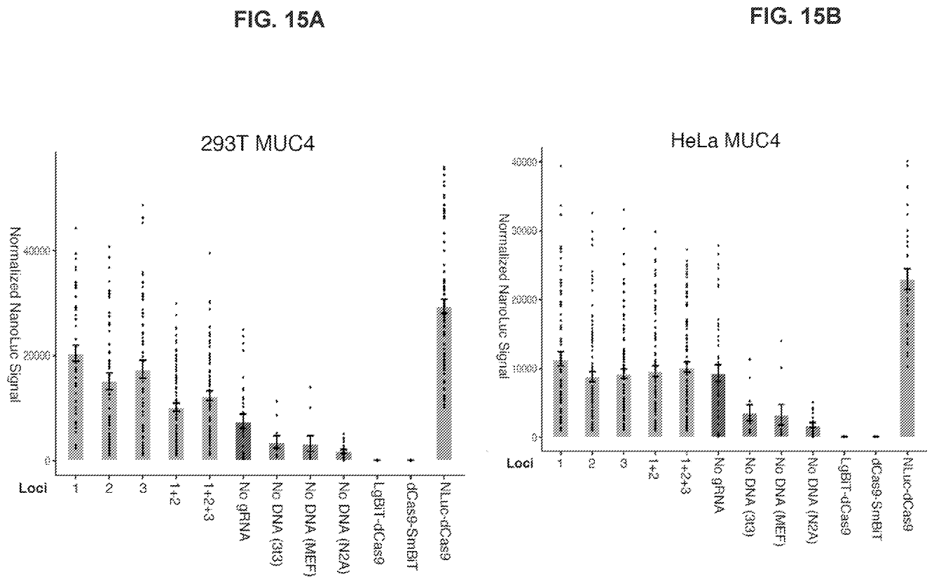

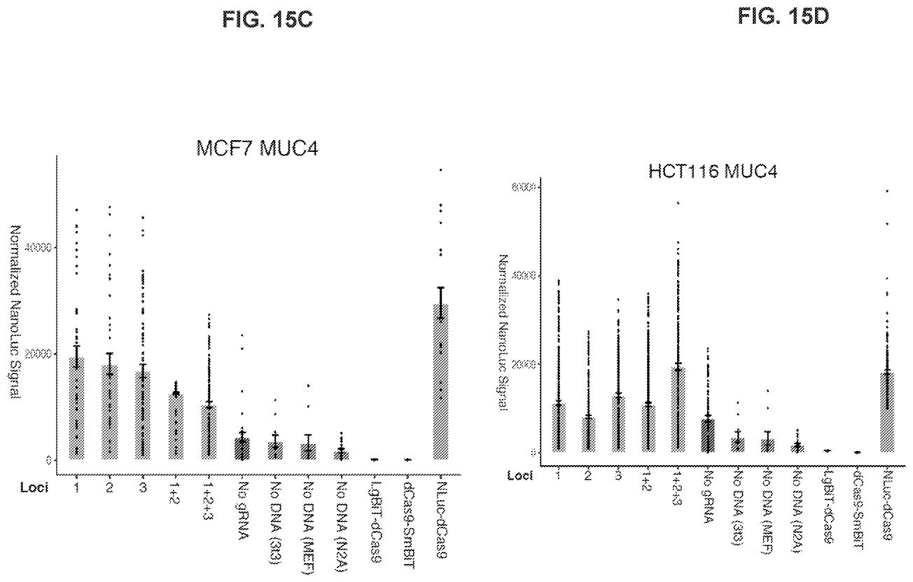

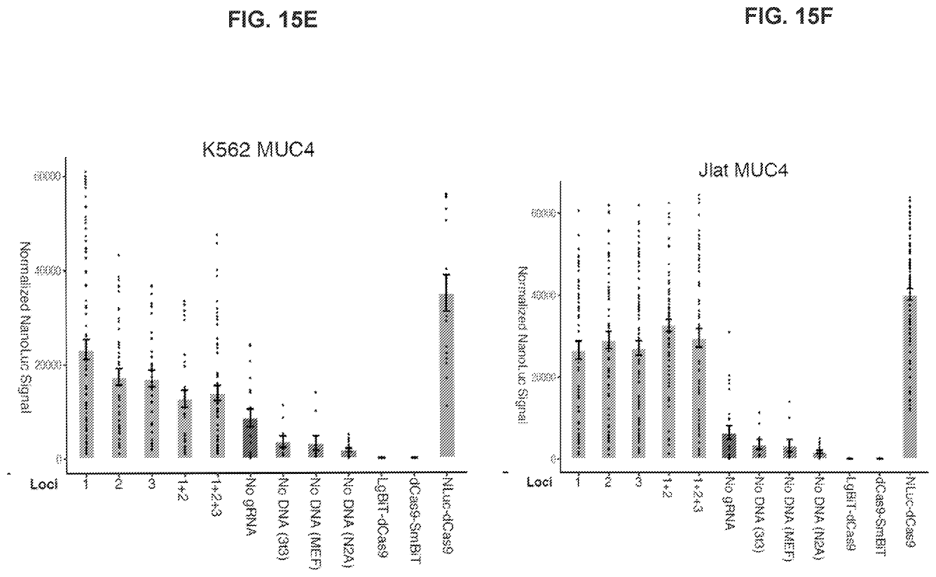

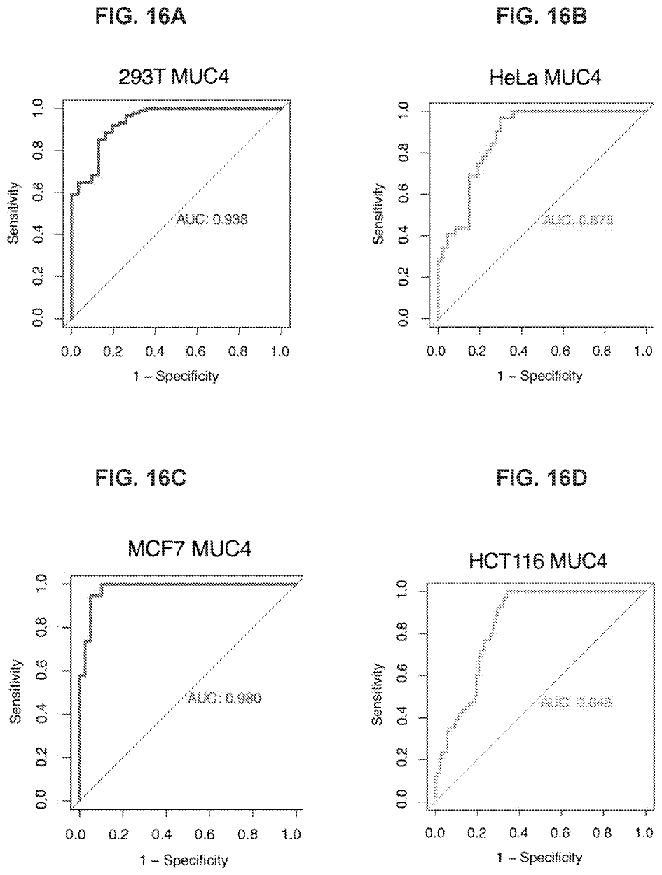

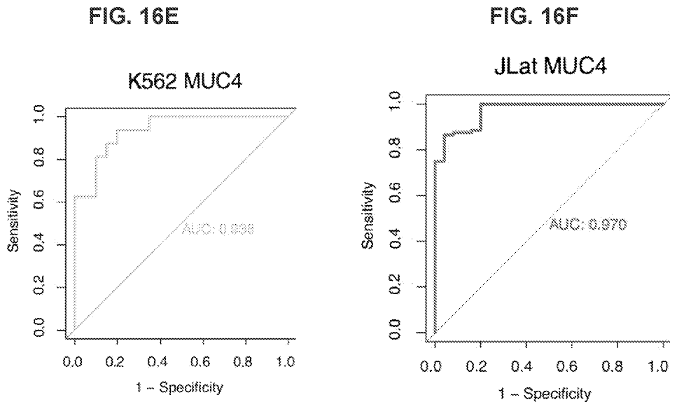

We therefore investigated if detection of unique sequences in MUC4 would be similarly correlative in both groups and individual cells, and if signal-to-noise would be dependent on cell type. Plasmid DNA encoding the split-probes, a GFP transfection reporter, and sgRNAs targeting 1, 2, or 3 unique loci (using 2, 4, or 6 sgRNAs) or combinations of these loci in MUC4 were transfected in to six different cell lines: HEK 293, HeLa, MCF7, HCT116, K563, and Jlat cells (FIG. 14). Again we observed substantial variation in the luminescence of individual cells, and background luminescence (no gRNA, luminescence due to auto-assembly) varied dramatically with cell type (FIGS. 15A-15B). However, receiver operating characteristic

(ROC) analysis demonstrated that this assay was an excellent discriminator of true positives from false positives, with most cell types displaying an area-under-the-curve (AUC) of >0.93. HeLa and HCT116 had AUC of >0.84, suggesting this was still a quite useful assay in these cell types.

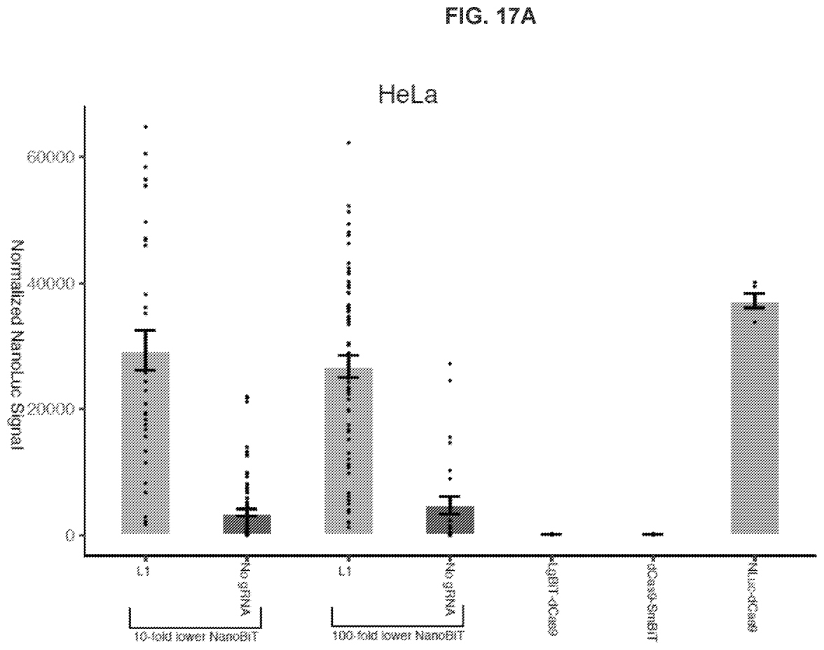

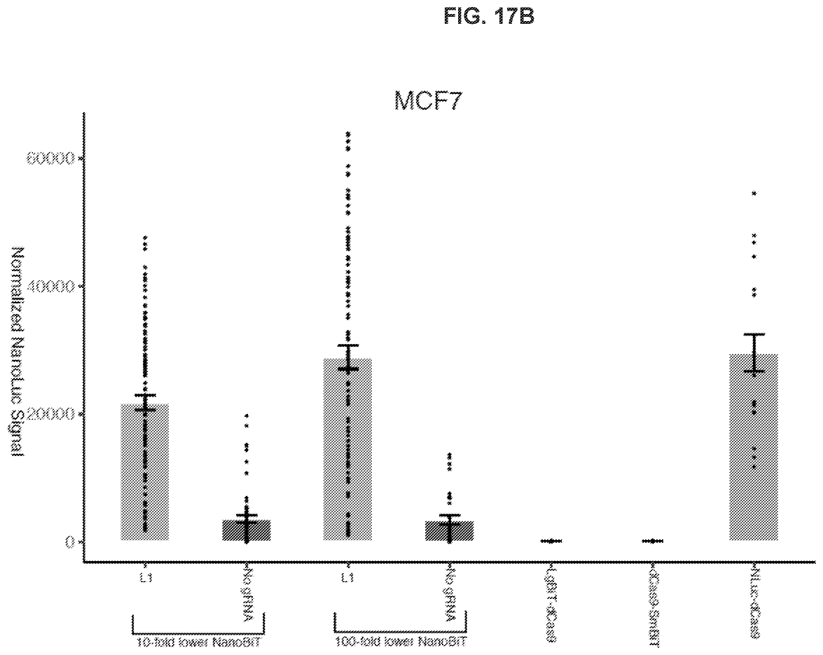

Moreover, we found that the signal-to-noise could be further improved by reducing the concentration of both the LgBiT-dCas9 and dCas9-SmBiT expression plasmids in the transfection mix by 10- or 100-fold (FIGS. 17A-17B). This result likely reflects that auto-assembly of the NLuc components was dependent on their concentration in the nucleus, thus the reduced concentrations were able to provide sufficient signal with dramatically reduced noise. Taken together, these data demonstrate the split NanoBiT probes as the first system capable of detecting unique DNA sequences in living human cells with exquisit sensitivity and specificity, using commonly available florescence microscopy.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

PARTIAL INFORMAL SEQUENCE LISTING
LgBiT-dCas9
SEQ ID NO: 1
MPKKKRKVGGSGGSYPYDVPDYAGGGSGGGSMVFTLEDFVGDWEQTAAYNLDQV
LEQGGVSSLLQNLAVSVTPIQRIVRSGENALKTDIHVIIPYEGLSADQMAQIEEVFKVV
YPVDDHHFKVILPYGTLVIDGVTPNMLNYFGRPYEGIAVFDGKKITVTGTLWNGNKI

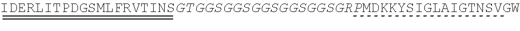

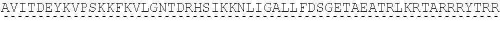

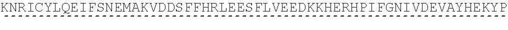

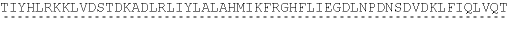

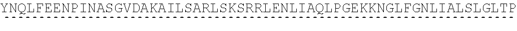

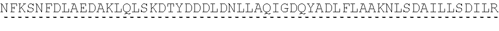

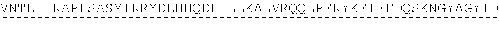

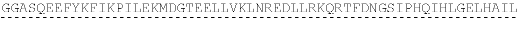

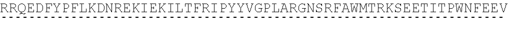

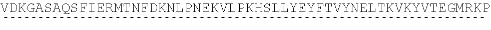

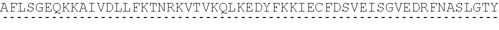

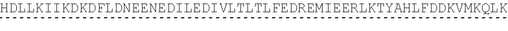

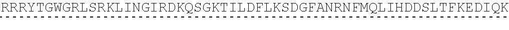

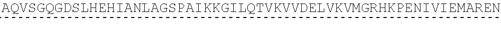

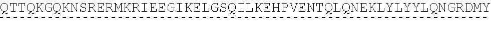

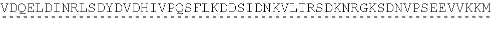

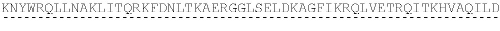

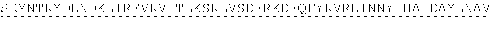

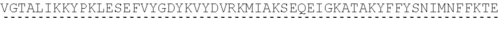

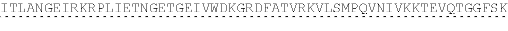

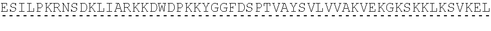

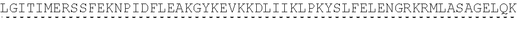

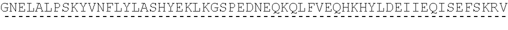

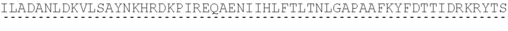

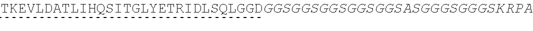

ATKKAGQAKKKKGGSGSGATNFSLLKQAGDVEENPGPAAA*

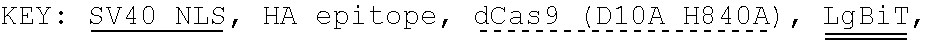

Nucleoplasmin NLS, P2A, variable length flexible linkers
SmBiT-dCas9
SEQ ID NO: 2
MPKKKRKVGGSGGSYPYDVPDYAGGGSGGGSMVTGYRLFEEILGTGGSGGSGGSGG

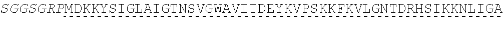

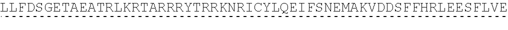

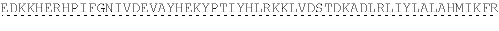

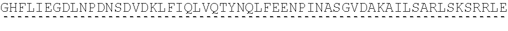

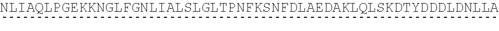

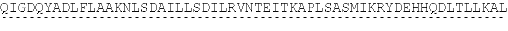

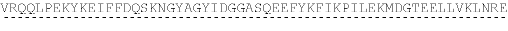

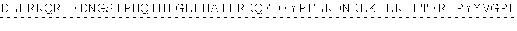

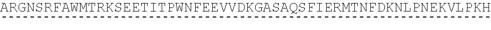

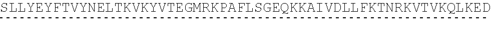

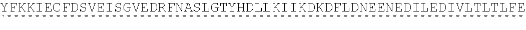

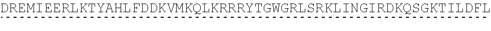

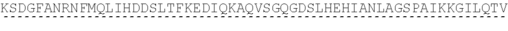

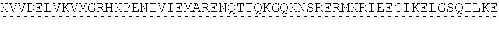

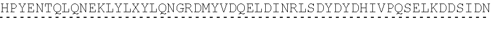

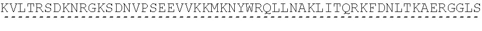

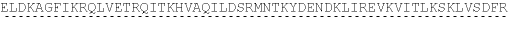

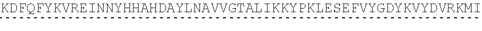

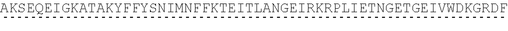

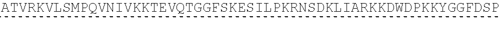

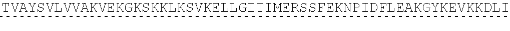

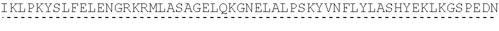

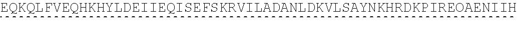

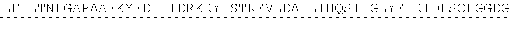

GSGGSGGSGGSGGSASGGGSGGGSKRPAATKKAGQAKKKKGGSGSGATNFSLLKQAG
DVEENPGPAAA*

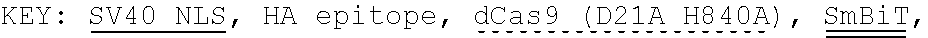

Nucleoplasmin NLS, P2A, variable length flexible linkers
dCas9-LgBiT
SEQ ID NO: 3
MPKKKRKVGGSGGSDYKDHDGDYKDHDIDYKDDDDKGGGSGGGSGTGGSGGSGGS

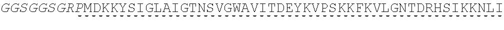

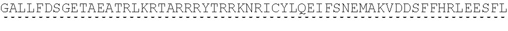

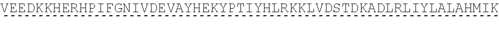

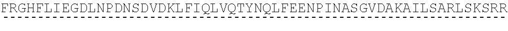

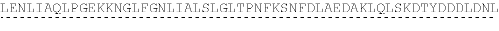

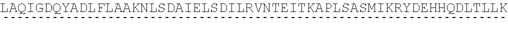

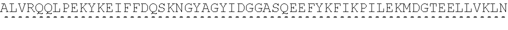

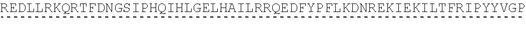

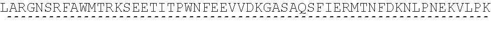

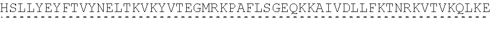

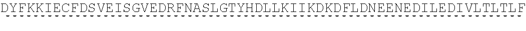

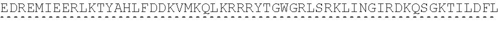

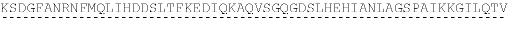

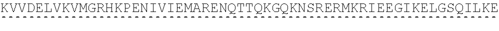

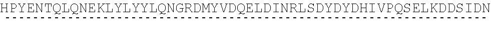

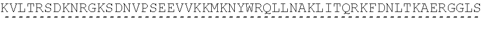

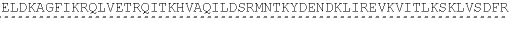

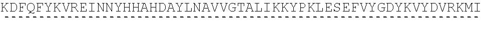

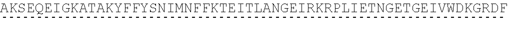

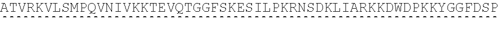

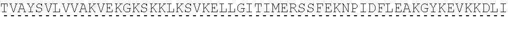

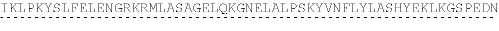

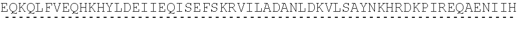

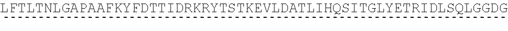

GSGGSGGSGGSGGSASVFTLEDFVGDWEQTAAYNLDQVLEQGGVSSLLQNLAVSVT
PIQRIVRSGENALKIDIHVIIPYEGLSADQMAQIEEVFKVVYPVDDHHFKVILPYGTLVI
DGVTPNMLNYFGRPYEGIAVFDGKKITVTGTLWNGNKIIDERLITPDGSMLFRVTINS
GGGSGGGSKRPAATKKAGQAKKKKGGSGSGAAA*

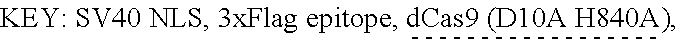

LgBiT, Nucleoplasmin NLS, variable length flexible linkers
dCas9-SmBiT:
SEQ ID NO: 4
MPKKKRKVGGSGGSDYKDHDGDYKDHDIDYKDDDDKGGGSGGGSGTGGSGGSGG
SGGSGGSGRPMDKKYSIGLAIGTNSVGWAVITDEYKVPSKKFKVLGNTDRHSIKKNL
IGALLFDSGETAEATRLKRTARRRYTRRKNRICYLQEIFSNEMAKVDDSFFHRLEESF
LVEEDKKHERHPIFGNIVDEVAYHEKYPTIYHLRKKLVDSTDKADLRLIYLALAHMI
KFRGHFLIEGDLNPDNSDVDKLFIQLVQTYNQLFEENPINASGVDAKAILSARLSKSR
RLENLIAQLPGEKKNGLFGNLIALSLGLTPNFKSNFDLAEDAKLQLSKDTYDDDLDN
LLAQIGDQYADLFLAAKNLSDAILLSDILRVNTEITKAPLSASMIKRYDEHHQDLTLL
KALVRQQLPEKYKEIFFDQSKNGYAGYIDGGASQEEFYKFIKPILEKMDGTEELLVKL
NREDLLRKQRTFDNGSIPHQIHLGELHAILRRQEDFYPFLKDNREKIEKILTFRIPYYV
GPLARGNSRFAWMTRKSEETITPWNFEEVVDKGASAQSFIERMTNFDKNLPNEKVLP
KHSLLYEYFTVYNELTKVKYVTEGMRKPAFLSGEQKKAIVDLLFKTNRKVTVKQLK
EDYFKKIECFDSVEISGVEDRFNASLGTYHDLLKIIKDKDFLDNEENEDILEDIVLTLTL
FEDREMIEERLKTYAHLFDDKVMKQLKRRRYTGWGRLSRKLINGIRDKQSGKTILDF
LKSDGFANRNFMQLIHDDSLTFKEDIQKAQVSGQGDSLHEHIANLAGSPAIKKGILQT
VKVVDELVKVMGRHKPENIVIEMARENQTTQKGQKNSRERMKRIEEGIKELGSQILK
EHPVENTQLQNEKLYLYYLQNGRDMYVDQELDINRLSDYDVDHIVPQSFLKDDSID
NKVLTRSDKNRGKSDNVPSEEVVKKMKNYWRQLLNAKLITQRKFDNLTKAERGGL
SELDKAGFIKRQLVETRQITKHVAQILDSRMNTKYDENDKLIREVKVITLKSKLVSDF
RKDFQFYKVREINNYHHAHDAYLNAVVGTALIKKYPKLESEFVYGDYKVYDVRKM
IAKSEQEIGKATAKYFFYSNIMNFFKTEITLANGEIRKRPLIETNGETGEIVWDKGRDF
ATVRKVLSMPQVNIVKKTEVQTGGFSKESILPKRNSDKLIARKKDWDPKKYGGFDSP
TVAYSVLVVAKVEKGKSKKLKSVKELLGITIMERSSFEKNPIDFLEAKGYKEVKKDLI
IKLPKYSLFELENGRKRMLASAGELQKGNELALPSKYVNFLYLASHYEKLKGSPEDN
EQKQLFVEQHKHYLDEIIEQISEFSKRVILADANLDKVLSAYNKHRDKPIREQAENIIH
LFTLTNLGAPAAFKYFDTTIDRKRYTSTKEVLDATLIHQSITGLYETRIDLSQLGGDG
GSGGSGGSGGSGGSASVTGYRLFEEILGGGSGGGSKRPAATKKAGQAKKKKGGSGSGA
AA*

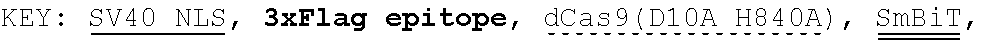

Nucleoplasmin NLS, variable length flexible linkers

1. A method of detecting the presence of a genomic sequence of interest in a living cell, the method comprising:
i) introducing a first fusion protein into the cell, the first fusion protein comprising an RNA-guided nuclease fused to a large fragment of NanoLuc luciferase (LgBiT);
ii) introducing a second fusion protein into the cell, the second fusion protein comprising an RNA-guided nuclease fused to a small fragment of NanoLuc luciferase (SmBiT);
iii) introducing a first and a second guide RNA into the cell, wherein the first and the second guide RNA are complementary to a first and a second nucleotide sequence within the genomic sequence of interest such that, in the presence of the genomic sequence of interest, when the first guide RNA is bound by the first fusion protein and the second guide RNA is bound by the second fusion protein, the guide RNAs direct the binding of the fusion proteins to the genomic sequence of interest such that the LgBiT and SmBiT elements are in proximity and luminescence is produced, indicating the presence of the genomic sequence of interest in the cell.

2. The method of claim 1, wherein the RNA-guided nuclease is dCas9.

3. The method of claim 2, wherein the first fusion protein is LgBiT-dCas9.

4. The method of claim 3, wherein the amino acid sequence of the first fusion protein is substantially (e.g., at least about 80%, 85%, 90%, 95%, 96%, 97%, 98%, 99% or more) identical to SEQ ID NO:1.

5. The method of claim 2, wherein the second fusion protein is dCas9-SmBiT.

6. The method of claim 5, wherein the amino acid sequence of the second fusion protein is substantially (e.g., at least about 80%, 85%, 90%, 95%, 96%, 97%, 98%, 99% or more) identical to SEQ ID NO:4.

7. The method of claim 1, wherein the first guide RNA and the first fusion protein, and the second guide RNA and the second fusion protein, are introduced into the cell as ribonucleoproteins (RNPs).

8. The method of claim 1, wherein the signal:noise ratio of the RFU/RLU in the presence of the first and second fusion proteins, the first and second guide RNAs, and the genomic sequence of interest relative to the RFU/RLU in the absence of any one or more of the first and second fusion proteins, the first and second guide RNAs, or the genomic sequence of interest is at least 2.5 , 5, 10, 15, 20, or 25.

9. The method of claim 1, wherein the first and second nucleotide sequences are arrayed in tandem or in inverse or everted orientation and are present within 50 nucleotides of one another.

10-13. (canceled)

14. The method of claim 1, wherein the second fusion protein is introduced at a molar excess relative to the first fusion protein.

15-16. (canceled)

17. The method of claim 1, wherein the cell is a eukaryotic cell.

18. The method of claim 17, wherein the eukaryotic cell is a mammalian cell.

19. The method of claim 18, wherein the mammalian cell is a human cell.

20. A cell comprising:
a first fusion protein comprising an RNA-guided nuclease fused to LgBiT;
a second fusion protein comprising an RNA-guided nuclease fused to SmBiT;
a first guide RNA that is complementary to a first nucleotide sequence within the genome and that can be bound by the first fusion protein and direct it to the first nucleotide sequence; and
a second guide RNA that is complementary to a second nucleotide sequence within the genome and that can be bound by the second fusion protein and direct it to the second nucleotide sequence;
wherein the first and the second nucleotide sequences are arranged in the genome such that when the first and second fusion proteins are directed to the first and second nucleotide sequences by the first and second guide RNAs, the LgBiT and SmBiT elements of the fusion proteins are brought into in proximity and luminescence is produced.

21. The cell of claim 20, wherein the RNA-guided nuclease is dCas9.

22. The cell of claim 21, wherein the first fusion protein is LgBiT-dCas9, or the second fusion protein is dCas9-SmBiT.

23-25. (canceled)

26. The cell of claim 20, wherein the first and second nucleotide sequences are arrayed in tandem or in inverse or everted orientation and are present within 50 nucleotides of one another.

27-28. (canceled)

29. The cell of claim 20, wherein the second fusion protein is present at a molar excess relative to the first fusion protein.

30-32. (canceled)

33. The cell of claim 20, wherein the cell is a mammalian cell.

34. (canceled)

35. A fusion protein comprising an RNA-guided nuclease and LgBiT or SmBiT.

36-42. (canceled)


SEQUENCE LISTING (135 sequences)

SEQ ID NO: 1 - protein, 1647 aa, Artificial Sequence
  Description of Artificial Sequence Synthetic polypeptide
MPKKKRKVGGSGGSYPYDVPDYAGGGSGGGSMVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGTGGSGGSGGSGGSGGSGRPMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGSGGSGGSGGSGGSASGGGSGGGSKRPAATKKAGQAKKKKGGSGSGATNFSLLKQAGDVEENPGPAAA

SEQ ID NO: 2 - protein, 1500 aa, Artificial Sequence
  Description of Artificial Sequence Synthetic polypeptide
MPKKKRKVGGSGGSYPYDVPDYAGGGSGGGSMVTGYRLFEEILGTGGSGGSGGSGGSGGSGRPMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGSGGSGGSGGSGGSASGGGSGGGSKRPAATKKAGQAKKKKGGSGSGATNFSLLKQAGDVEENPGPAAA

SEQ ID NO: 3 - protein, 1640 aa, Artificial Sequence
  Description of Artificial Sequence Synthetic polypeptide
MPKKKRKVGGSGGSDYKDHDGDYKDHDIDYKDDDDKGGGSGGGSGTGGSGGSGGSGGSGGSGRPMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGSGGSGGSGGSGGSASVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGGGSGGGSKRPAATKKAGQAKKKKGGSGSGAAA

SEQ ID NO: 4 - protein, 1493 aa, Artificial Sequence
  Description of Artificial Sequence Synthetic polypeptide
MPKKKRKVGGSGGSDYKDHDGDYKDHDIDYKDDDDKGGGSGGGSGTGGSGGSGGSGGSGGSGRPMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGSGGSGGSGGSGGSASVTGYRLFEEILGGGSGGGSKRPAATKKAGQAKKKKGGSGSGAAA

SEQ ID NO: 5 - protein, 15 aa, Artificial Sequence
  Description of Artificial Sequence Synthetic peptide
GGSGGSGGSGGSGGS

SEQ ID NO: 6 - DNA, 675 bp, Artificial Sequence
  Description of Artificial Sequence Synthetic polynucleotide
tccatagaagacaccgggaccgatccagcctccggactctagaggatcgaacccttgccaccatgcccaagaagaagaggaaggtgggaggctccggaggaagctacccatacgatgtcccagactacgcgggtggcgggtccggcggtggatccatggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagtggtaccggagggagtggtggaagcggcggttctggtggctcag

SEQ ID NO: 7 - DNA, 234 bp, Artificial Sequence
  Description of Artificial Sequence Synthetic polynucleotide
tccatagaagacaccgggaccgatccagcctccggactctagaggatcgaacccttgccaccatgcccaagaagaagaggaaggtgggaggctccggaggaagctacccatacgatgtcccagactacgcgggtggcgggtccggcggtggatccatggtgaccggctaccggctgttcgaggagattctcggtaccggagggagtggtggaagcggcggttctggtggctcag

SEQ ID NO: 8 - DNA, 654 bp, Artificial Sequence
  Description of Artificial Sequence Synthetic polynucleotide
tagtggaggttcaggaggatccggggggagcggagggagcgctagcgtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagcggtggaggctccggaggtggatctaaaaggccggcggccacgaaaaaggccggtcaggcaaaaaagaaaaagggtggtagtggaagcggagcggccgcatgaaagggttcgatccctaccggttagtaatgagt

SEQ ID NO: 9 - DNA, 213 bp, Artificial Sequence
  Description of Artificial Sequence Synthetic polynucleotide
tagtggaggttcaggaggatccggggggagcggagggagcgctagcgtgaccggctaccggctgttcgaggagattctgggtggaggctccggaggtggatctaaaaggccggcggccacgaaaaaggccggtcaggcaaaaaagaaaaagggtggtagtggaagcggagcggccgcatgaaagggttcgatccctaccggttagtaatgagt

SEQ ID NO: 10 - DNA, 20 bp, Artificial Sequence
  Description of Artificial Sequence Synthetic primer
tccatagaagacaccgggac

SEQ ID NO: 11 - DNA, 44 bp, Artificial Sequence
  Description of Artificial Sequence Synthetic primer
cgaacagccggtagccggtcacactgttgatggttactcggaac

SEQ ID NO: 12 - DNA, 44 bp, Artificial Sequence
  Description of Artificial Sequence Synthetic primer
gttccgagtaaccatcaacagtgtgaccggctaccggctgttcg

SEQ ID NO: 13 - DNA, 21 bp, Artificial Sequence
  Description of Artificial Sequence Synthetic primer
ctgagccaccagaaccgccgc

SEQ ID NO: 14 - protein, 1658 aa, Artificial Sequence
  Description of Artificial Sequence Synthetic polypeptide
MPKKKRKVGGSGGSYPYDVPDYAGGGSGGGSMVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSVTGYRLFEEILGTGGSGGSGGSGGSGGSGRPMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGSGGSGGSGGSGGSASGGGSGGGSKRPAATKKAGQAKKKKGGSGSGATNFSLLKQAGDVEENPGPAAA

SEQ ID NO: 15 - DNA, 20 bp, Artificial Sequence
  Description of Artificial Sequence Synthetic oligonucleotide
gctccctacgcatgcgtccc

SEQ ID NO: 16 - DNA, 23 bp, Unknown
  Description of Unknown Target sequence
gctccctacgcatgcgtcccagg

SEQ ID NO: 17 - DNA, 20 bp, Artificial Sequence
  Description of Artificial Sequence Synthetic oligonucleotide
gatggctcaggtttgtcgcg

SEQ ID NO: 18 - DNA, 23 bp, Unknown
  Description of Unknown Target sequence
gatggctcaggtttgtcgcgcgg

SEQ ID NO: 19 - DNA, 60 bp, Artificial Sequence
  Description of Artificial Sequence Synthetic oligonucleotide
  modified_base (42)..(60): a, c, t, g, unknown or other
tttcttggctttatatatcttgtggaaaggacgaaacaccgnnnnnnnnnnnnnnnnnnn

SEQ ID NO: 20 - DNA, 60 bp, Artificial Sequence
  Description of Artificial Sequence Synthetic oligonucleotide
  modified_base (41)..(59): a, c, t, g, unknown or other
gactagccttattttaacttgctatttctagctctaaaacnnnnnnnnnnnnnnnnnnnc

SEQ ID NO: 21 - DNA, 60 bp, Artificial Sequence
  Description of Artificial Sequence Synthetic oligonucleotide
tttcttggctttatatatcttgtggaaaggacgaaacaccgctccctacgcatgcgtccc

SEQ ID NO: 22 - DNA, 60 bp, Artificial Sequence
  Description of Artificial Sequence Synthetic oligonucleotide
gactagccttattttaacttgctatttctagctctaaaacgggacgcatgcgtagggagc

SEQ ID NO: 23 - DNA, 60 bp, Artificial Sequence
  Description of Artificial Sequence Synthetic oligonucleotide
tttcttggctttatatatcttgtggaaaggacgaaacaccgatggctcaggtttgtcgcg

SEQ ID NO: 24 - DNA, 60 bp, Artificial Sequence
  Description of Artificial Sequence Synthetic oligonucleotide
gactagccttattttaacttgctatttctagctctaaaaccgcgacaaacctgagccatc

SEQ ID NO: 25 - DNA, 597 bp, Artificial Sequence
  Description of Artificial Sequence Synthetic polynucleotide
gggtttgctgctcatctatactttcacaatcttgagctgcagggcaaagagctccctacgcatgcgtcccaggcagcgtgtatagtgaaaaggaacccggggatggaggaagggacatagggagatggctcaggtttgtcgcgcggtatgtagcatggcccgggaagtacagtagagctccctacgcatgcgtcccaggtgctacttacatattctcccgggtaaattaattcttatgagatggctcaggtttgtcgcgcggctagtagcccggggcattgtgctccctacgcatgcgtcccaggatctaatcatatcccgggatgaaggtctatgatggctcaggtttgtcgcgcggtatgctgaataattgagcccgggatagtgaaatttatgatgctccctacgcatgcgtcccaggtgcttttcccgggtgcacaagatggctcaggtttgtcgcgcggaatataataatatgtagatggtcccgggtaggttgttatacatttactgagctccctacgcatgcgtcccaggtttgtagaaggctaggggaacaggttagtttgagggaattctaatggatccttctatggg

SEQ ID NO: 26 - DNA, 18 bp, Artificial Sequence
  Description of Artificial Sequence Synthetic primer
cttgagctgcagggcaaa

SEQ ID NO: 27 - DNA, 38 bp, Artificial Sequence
  Description of Artificial Sequence Synthetic primer
cgacaaacctgagccatctccctgcctgggacgcatgc

SEQ ID NO: 28 - DNA, 38 bp, Artificial Sequence
  Description of Artificial Sequence Synthetic primer
gcatgcgtcccaggcagggagatggctcaggtttgtcg

SEQ ID NO: 29 - DNA, 18 bp, Artificial Sequence
  Description of Artificial Sequence Synthetic primer
ttcccgggccatgctaca

SEQ ID NO: 30 - DNA, 92 bp, Artificial Sequence
  Description of Artificial Sequence Synthetic oligonucleotide
cttgagctgcagggcaaagagctccctacgcatgcgtcccaggcagggagatggctcaggtttgtcgcgcggtatgtagcatggcccgggaa

SEQ ID NO: 31 - DNA, 37 bp, Artificial Sequence
  Description of Artificial Sequence Synthetic primer
caaacctgagccatctccctcgctgcctgggacgcat

SEQ ID NO: 32 - DNA, 37 bp, Artificial Sequence
  Description of Artificial Sequence Synthetic primer
atgcgtcccaggcagcgagggagatggctcaggtttg

SEQ ID NO: 33 - DNA, 96 bp, Artificial Sequence
  Description of Artificial Sequence Synthetic oligonucleotide
cttgagctgcagggcaaagagctccctacgcatgcgtcccaggcagcgagggagatggctcaggtttgtcgcgcggtatgtagcatggcccgggaa

SEQ ID NO: 34 - DNA, 39 bp, Artificial Sequence
  Description of Artificial Sequence Synthetic primer
caaacctgagccatctccctatacacgctgcctgggacg

SEQ ID NO: 35 - DNA, 39 bp, Artificial Sequence
  Description of Artificial Sequence Synthetic primer
cgtcccaggcagcgtgtatagggagatggctcaggtttg

SEQ ID NO: 36 - DNA, 101 bp, Artificial Sequence
  Description of Artificial Sequence Synthetic polynucleotide
cttgagctgcagggcaaagagctccctacgcatgcgtcccaggcagcgtgtatagggagatggctcaggtttgtcgcgcggtatgtagcatggcccgggaa

SEQ ID NO: 37 - DNA, 20 bp, Artificial Sequence
  Description of Artificial Sequence Synthetic primer
gggatagtgaaatttatgat

SEQ ID NO: 38 - DNA, 25 bp, Artificial Sequence
  Description of Artificial Sequence Synthetic primer
gggaccatctacatattattatatt

SEQ ID NO: 39 - DNA, 111 bp, Artificial Sequence
  Description of Artificial Sequence Synthetic polynucleotide
gggatagtgaaatttatgatgctccctacgcatgcgtcccaggtgcttttcccgggtgcacaagatggctcaggtttgtcgcgcggaatataataatatgtagatggtccc

SEQ ID NO: 40 - DNA, 39 bp, Artificial Sequence
  Description of Artificial Sequence Synthetic primer
gagccatctccctatgtccctctatacacgctgcctggg

SEQ ID NO: 41 - DNA, 39 bp, Artificial Sequence
  Description of Artificial Sequence Synthetic primer
cccaggcagcgtgtatagagggacatagggagatggctc

SEQ ID NO: 42 - DNA, 111 bp, Artificial Sequence
  Description of Artificial Sequence Synthetic polynucleotide
cttgagctgcagggcaaagagctccctacgcatgcgtcccaggcagcgtgtatagagggacatagggagatggctcaggtttgtcgcgcggtatgtagcatggcccgggaa

SEQ ID NO: 43 - DNA, 18 bp, Artificial Sequence
  Description of Artificial Sequence Synthetic primer
ctagtagcccggggcatt

SEQ ID NO: 44 - DNA, 20 bp, Artificial Sequence
  Description of Artificial Sequence Synthetic primer
gggctcaattattcagcata

SEQ ID NO: 45 - DNA, 116 bp, Artificial Sequence
  Description of Artificial Sequence Synthetic polynucleotide
ctagtagcccggggcattgtgctccctacgcatgcgtcccaggatctaatcatatcccgggatgaaggtctatgatggctcaggtttgtcgcgcggtatgctgaataattgagccc

SEQ ID NO: 46 - DNA, 45 bp, Artificial Sequence
  Description of Artificial Sequence Synthetic primer
gccatctccctatgtcccttcctttttcactatacacgctgcctg

SEQ ID NO: 47 - DNA, 45 bp, Artificial Sequence
  Description of Artificial Sequence Synthetic primer
caggcagcgtgtatagtgaaaaaggaagggacatagggagatggc

SEQ ID NO: 48 - DNA, 121 bp, Artificial Sequence
  Description of Artificial Sequence Synthetic polynucleotide
cttgagctgcagggcaaagagctccctacgcatgcgtcccaggcagcgtgtatagtgaaaaaggaagggacatagggagatggctcaggtttgtcgcgcggtatgtagcatggcccgggaa

SEQ ID NO: 49 - DNA, 20 bp, Artificial Sequence
  Description of Artificial Sequence Synthetic primer
ggcccgggaagtacagtaga

SEQ ID NO: 50 - DNA, 20 bp, Artificial Sequence
  Description of Artificial Sequence Synthetic primer
acaatgccccgggctactag

SEQ ID NO: 51 - DNA, 126 bp, Artificial Sequence
  Description of Artificial Sequence Synthetic polynucleotide
ggcccgggaagtacagtagagctccctacgcatgcgtcccaggtgctacttacatattctcccgggtaaattaattcttatgagatggctcaggtttgtcgcgcggctagtagcccggggcattgt

SEQ ID NO: 52 - DNA, 136 bp, Artificial Sequence
  Description of Artificial Sequence Synthetic polynucleotide
cttgagctgcagggcaaagagctccctacgcatgcgtcccaggcagcgtgtatagtgaaaaggaacccggggatggaggaagggacatagggagatggctcaggtttgtcgcgcggtatgtagcatggcccgggaa

SEQ ID NO: 53 - DNA, 647 bp, Artificial Sequence
  Description of Artificial Sequence Synthetic polynucleotide
gggtttgctgctcatctatactttcacaatcttgagctgcagggcaaagactacaatgggattaataaattgtactctaaaggatattgaaaacttgtgagctccctacgcatgcgtcccaggcagcgtgtatagtgaaaaggaacccggggatggaggaagggacatagggaccgcgcgacaaacctgagccatctatgtagcatggcccgggaagtacagtagagctccctacgcatgcgtcccaggtgctacttacatattctcccgggtaaattaattcttatgaccgcgcgacaaacctgagccatcctagtagcccggggcattgtgctccctacgcatgcgtcccaggatctaatcatatcccgggatgaaggtctatccgcgcgacaaacctgagccatctatgctgaataattgagcccgggatagtgaaatttatgatgctccctacgcatgcgtcccaggtgcttttcccgggtgcacaaccgcgcgacaaacctgagccatcaatataataatatgtagatggtcccgggtaggttgttatacatttactgagctccctacgcatgcgtcccaggtttgtagaaggctaggggaacaggttagtttgagggaattctaatggatccttctatggg

SEQ ID NO: 54 - DNA, 27 bp, Artificial Sequence
  Description of Artificial Sequence Synthetic primer
actctaaaggatattgaaaacttgtga

SEQ ID NO: 55 - DNA, 34 bp, Artificial Sequence
  Description of Artificial Sequence Synthetic primer
aggtttgtcgcgcggtccctgcctgggacgcatg

SEQ ID NO: 56 - DNA, 34 bp, Artificial Sequence
  Description of Artificial Sequence Synthetic primer
catgcgtcccaggcagggaccgcgcgacaaacct

SEQ ID NO: 57 - DNA, 18 bp, Artificial Sequence
  Description of Artificial Sequence Synthetic primer
actgtacttcccgggcca

SEQ ID NO: 58 - DNA, 106 bp, Artificial Sequence
  Description of Artificial Sequence Synthetic polynucleotide
actctaaaggatattgaaaacttgtgagctccctacgcatgcgtcccaggcagggaccgcgcgacaaacctgagccatctatgtagcatggcccgggaagtacagt

SEQ ID NO: 59 - DNA, 35 bp, Artificial Sequence
  Description of Artificial Sequence Synthetic primer
gtttgtcgcgcggtccctcgctgcctgggacgcat

SEQ ID NO: 60 - DNA, 35 bp, Artificial Sequence
  Description of Artificial Sequence Synthetic primer
atgcgtcccaggcagcgagggaccgcgcgacaaac

SEQ ID NO: 61 - DNA, 110 bp, Artificial Sequence
  Description of Artificial Sequence Synthetic polynucleotide
actctaaaggatattgaaaacttgtgagctccctacgcatgcgtcccaggcagcgagggaccgcgcgacaaacctgagccatctatgtagcatggcccgggaagtacagt

SEQ ID NO: 62 - DNA, 36 bp, Artificial Sequence
  Description of Artificial Sequence Synthetic primer
gtttgtcgcgcggtccctatacacgctgcctgggac

SEQ ID NO: 63 - DNA, 36 bp, Artificial Sequence
  Description of Artificial Sequence Synthetic primer
gtcccaggcagcgtgtatagggaccgcgcgacaaac

SEQ ID NO: 64 - DNA, 115 bp, Artificial Sequence
  Description of Artificial Sequence Synthetic polynucleotide
actctaaaggatattgaaaacttgtgagctccctacgcatgcgtcccaggcagcgtgtatagggaccgcgcgacaaacctgagccatctatgtagcatggcccgggaagtacagt

SEQ ID NO: 65 - DNA, 111 bp, Artificial Sequence
  Description of Artificial Sequence Synthetic polynucleotide
gggatagtgaaatttatgatgctccctacgcatgcgtcccaggtgcttttcccgggtgcacaaccgcgcgacaaacctgagccatcaatataataatatgtagatggtccc

SEQ ID NO: 66 - DNA, 34 bp, Artificial Sequence
  Description of Artificial Sequence Synthetic primer
gcggtccctatgtccctctatacacgctgcctgg

SEQ ID NO: 67 - DNA, 34 bp, Artificial Sequence
  Description of Artificial Sequence Synthetic primer
ccaggcagcgtgtatagagggacatagggaccgc

SEQ ID NO: 68 - DNA, 125 bp, Artificial Sequence
  Description of Artificial Sequence Synthetic polynucleotide
actctaaaggatattgaaaacttgtgagctccctacgcatgcgtcccaggcagcgtgtatagagggacatagggaccgcgcgacaaacctgagccatctatgtagcatggcccgggaagtacagt

SEQ ID NO: 69 - DNA, 116 bp, Artificial Sequence
  Description of Artificial Sequence Synthetic polynucleotide
ctagtagcccggggcattgtgctccctacgcatgcgtcccaggatctaatcatatcccgggatgaaggtctatccgcgcgacaaacctgagccatctatgctgaataattgagccc

SEQ ID NO: 70 - DNA, 40 bp, Artificial Sequence
  Description of Artificial Sequence Synthetic primer
ggtccctatgtcccttcctttttcactatacacgctgcct

SEQ ID NO: 71 - DNA, 40 bp, Artificial Sequence
  Description of Artificial Sequence Synthetic primer
aggcagcgtgtatagtgaaaaaggaagggacatagggacc

SEQ ID NO: 72 - DNA, 135 bp, Artificial Sequence
  Description of Artificial Sequence Synthetic polynucleotide
actctaaaggatattgaaaacttgtgagctccctacgcatgcgtcccaggcagcgtgtatagtgaaaaaggaagggacatagggaccgcgcgacaaacctgagccatctatgtagcatggcccgggaagtacagt

SEQ ID NO: 73 - DNA, 126 bp, Artificial Sequence
  Description of Artificial Sequence Synthetic polynucleotide
ggcccgggaagtacagtagagctccctacgcatgcgtcccaggtgctacttacatattctcccgggtaaattaattcttatgaccgcgcgacaaacctgagccatcctagtagcccggggcattgt

SEQ ID NO: 74 - DNA, 150 bp, Artificial Sequence
  Description of Artificial Sequence Synthetic polynucleotide
actctaaaggatattgaaaacttgtgagctccctacgcatgcgtcccaggcagcgtgtatagtgaaaaggaacccggggatggaggaagggacatagggaccgcgcgacaaacctgagccatctatgtagcatggcccgggaagtacagt

SEQ ID NO: 75 - DNA, 22 bp, Artificial Sequence
  Description of Artificial Sequence Synthetic primer
ccgggtaaattaattcttatga

SEQ ID NO: 76 - DNA, 36 bp, Artificial Sequence
  Description of Artificial Sequence Synthetic primer
gcatgcgtagggagcacataggatggctcaggtttg

SEQ ID NO: 77 - DNA, 36 bp, Artificial Sequence
  Description of Artificial Sequence Synthetic primer
caaacctgagccatcctatgtgctccctacgcatgc

SEQ ID NO: 78 - DNA, 22 bp, Artificial Sequence
  Description of Artificial Sequence Synthetic primer
atctaatcatatcccgggatga

SEQ ID NO: 79 - DNA, 96 bp, Artificial Sequence
  Description of Artificial Sequence Synthetic oligonucleotide
ccgggtaaattaattcttatgaccgcgcgacaaacctgagccatcctatgtgctccctacgcatgcgtcccaggatctaatcatatcccgggatga

SEQ ID NO: 80 - DNA, 36 bp, Artificial Sequence
  Description of Artificial Sequence Synthetic primer
atgcgtagggagcacaatactaggatggctcaggtt

SEQ ID NO: 81 - DNA, 36 bp, Artificial Sequence
  Description of Artificial Sequence Synthetic primer
aacctgagccatcctagtattgtgctccctacgcat

SEQ ID NO: 82 - DNA, 100 bp, Artificial Sequence
  Description of Artificial Sequence Synthetic polynucleotide
ccgggtaaattaattcttatgaccgcgcgacaaacctgagccatcctagtattgtgctccctacgcatgcgtcccaggatctaatcatatcccgggatga

SEQ ID NO: 83 - DNA, 36 bp, Artificial Sequence
  Description of Artificial Sequence Synthetic primer
cgtagggagcacaatgccctactaggatggctcagg

SEQ ID NO: 84 - DNA, 36 bp, Artificial Sequence
  Description of Artificial Sequence Synthetic primer
cctgagccatcctagtagggcattgtgctccctacg

SEQ ID NO: 85 - DNA, 105 bp, Artificial Sequence
  Description of Artificial Sequence Synthetic polynucleotide
ccgggtaaattaattcttatgaccgcgcgacaaacctgagccatcctagtagggcattgtgctccctacgcatgcgtcccaggatctaatcatatcccgggatga

SEQ ID NO: 86 - DNA, 110 bp, Artificial Sequence
  Description of Artificial Sequence Synthetic polynucleotide
ccgggtaaattaattcttatgaccgcgcgacaaacctgagccatcctagtagcccggggcattgtgctccctacgcatgcgtcccaggatctaatcatatcccgggatga

SEQ ID NO: 87 - DNA, 27 bp, Artificial Sequence
  Description of Artificial Sequence Synthetic primer
ttctcccgggtaaattaattcttatga

SEQ ID NO: 88 - DNA, 36 bp, Artificial Sequence
  Description of Artificial Sequence Synthetic primer
gggagcacaatgccccggcccgggctactaggatgg

SEQ ID NO: 89 - DNA, 36 bp, Artificial Sequence
  Description of Artificial Sequence Synthetic primer
ccatcctagtagcccgggccggggcattgtgctccc

SEQ ID NO: 90 - DNA, 27 bp, Artificial Sequence
  Description of Artificial Sequence Synthetic primer
gaccttcatcccgggatatgattagat

SEQ ID NO: 91 - DNA, 125 bp, Artificial Sequence
  Description of Artificial Sequence Synthetic polynucleotide
ttctcccgggtaaattaattcttatgaccgcgcgacaaacctgagccatcctagtagcccgggccggggcattgtgctccctacgcatgcgtcccaggatctaatcatatcccgggatgaaggtc

SEQ ID NO: 92 - DNA, 20 bp, Artificial Sequence
  Description of Artificial Sequence Synthetic primer
gatggaggaagggacatagg

SEQ ID NO: 93 - DNA, 22 bp, Artificial Sequence
  Description of Artificial Sequence Synthetic primer
ccgggagaatatgtaagtagca

SEQ ID NO: 94 - DNA, 120 bp, Artificial Sequence
  Description of Artificial Sequence Synthetic polynucleotide
gatggaggaagggacatagggaccgcgcgacaaacctgagccatctatgtagcatggcccgggaagtacagtagagctccctacgcatgcgtcccaggtgctacttacatattctcccgg

SEQ ID NO: 95 - DNA, 41 bp, Artificial Sequence
  Description of Artificial Sequence Synthetic primer
agctctactgtacttcccgggccgggccatgctacatagat

SEQ ID NO: 96 - DNA, 41 bp, Artificial Sequence
  Description of Artificial Sequence Synthetic primer
atctatgtagcatggcccggcccgggaagtacagtagagct

SEQ ID NO: 97 - DNA, 125 bp, Artificial Sequence
  Description of Artificial Sequence Synthetic polynucleotide
gatggaggaagggacatagggaccgcgcgacaaacctgagccatctatgtagcatggcccggcccgggaagtacagtagagctccctacgcatgcgtcccaggtgctacttacatattctcccgg

SEQ ID NO: 98 - DNA, 20 bp, Artificial Sequence
  Description of Artificial Sequence Synthetic primer
atcccgggatgaaggtctat

SEQ ID NO: 99 - DNA, 17 bp, Artificial Sequence
  Description of Artificial Sequence Synthetic primer
ttgtgcacccgggaaaa

SEQ ID NO: 100 - DNA, 126 bp, Artificial Sequence
  Description of Artificial Sequence Synthetic polynucleotide
atcccgggatgaaggtctatccgcgcgacaaacctgagccatctatgctgaataattgagcccgggatagtgaaatttatgatgctccctacgcatgcgtcccaggtgcttttcccgggtgcacaa

SEQ ID NO: 101 - DNA, 18 bp, Artificial Sequence
  Description of Artificial Sequence Synthetic primer
ttttcccgggtgcacaac

SEQ ID NO: 102 - DNA, 23 bp, Artificial Sequence
  Description of Artificial Sequence Synthetic primer
gttcccctagccttctacaaacc

SEQ ID NO: 103 - DNA, 134 bp, Artificial Sequence
  Description of Artificial Sequence Synthetic polynucleotide
ttttcccgggtgcacaaccgcgcgacaaacctgagccatcaatataataatatgtagatggtcccgggtaggttgttatacatttactgagctccctacgcatgcgtcccaggtttgtagaaggctaggggaac

SEQ ID NO: 104 - DNA, 51 bp, Homo sapiens
gccacccctcttcctgtcaccgacacttcctcagcatccacaggtcacgcc

SEQ ID NO: 105 - DNA, 51 bp, Homo sapiens
ggcgtgacctgtggatgctgaggaagtgtcggtgacaggaagaggggtggc

SEQ ID NO: 106 - DNA, 23 bp, Artificial Sequence
  Description of Artificial Sequence Synthetic oligonucleotide
ggcgtgacctgtggatgctgagg

SEQ ID NO: 107 - DNA, 23 bp, Homo sapiens
gacacttcctcagcatccacagg

SEQ ID NO: 108 - DNA, 23 bp, Homo sapiens
ggtggatgctgaggaagtgtcgg

SEQ ID NO: 109 - DNA, 23 bp, Homo sapiens
ggtgaggaagtgtcggtgacagg

SEQ ID NO: 110 - DNA, 23 bp, Homo sapiens
gaagtgtcggtgacaggaagagg

SEQ ID NO: 111 - DNA, 23 bp, Homo sapiens
ggtgtcggtgacaggaagagggg

SEQ ID NO: 112 - DNA, 23 bp, Homo sapiens
ggcggtgacaggaagaggggtgg

SEQ ID NO: 113 - DNA, 5627 bp, Homo sapiens
atgaagggggcacgctggaggagggtcccctgggtgtccctgagctgcctgtgtctctgcctccttccgcatgtggtcccaggtaagtgatggagacagcagatgaggctggctgcggggagcacttgggggaggtgggagctgtcagagaaagaggtccggggagacagagagagagagagagagaataggggaaagggagacagcgaagaggaagagaagggagagaaaaagagggagagggaaaggagaaagagatgaatgggacaacatggggggaaggtggagagagacccagagagggaaagaagaggaagagaagagggagagagaaagaagagtggaggccgtgcgcggtggctcatgcctgtaatcccagcactttcggaggccaaggcaggagatcacctgaggtcaggagttcgagaccagcctggccgacatggtgaaaccccgtctctactaaatatacaaaaattagccggtcgtggtgggccccacctgtaattccagctactcaggagtctgaggcaggagaatcacttgaacctgggaggtggaggttgcagtgagccaagatcgcgccactgcactccagcctgggagagagagcgagactctgtctcaaaataaataaataaataaataaataaataaataaataaataaatataaaataaaataaaaaataagaagaggagaaaagtggggaagaggaggcatgaactggcagatacgggacaagatctgaggggaaagacagagggagaatgctcgaaagagagagaaaagagaacagagggccagagagcagcccggcgatgtctggaaggatccatggtgagagcccaggcttactcgcagagagaaagacaggcagagccagagcaagaggaacagagtcaaggagaaagatgtacacccttgtgtacagagctgggggtagaggggatgccaggaaagctgggtgatggagacggaagaaaactcatgtaaagctgcagggtgagaggacgagacaggtgagacgcagacaaactgaggaccctgggaatggagagaggagaagatcgggagacagcagcaagcaagggaagcgacaaggaggagaggggcaggccggccgggagggtggtgcggaggaggcggccagggcgcagagggccgggaggtgctggccgtgggcttcttacctctgagctcgggtttaaaagcctccatttgggtcacggccttgcctggggctcgtagccccggcattggccttgggctcctccgtgtacagagctgggaggggagggatgccaggcctgtgggagatgttccctcgggggcccccgtcctcttccccacactttccaggctgtccctctggcttcaggaccaagttttattctgtgtttctgggtgtctgagtctttgggggagagtctggggtccagagttcaagctggggttagagtctcagctcctgccctgcctctcagcaggctaagaacagtcgccgagggaaaatatttcttgggcgcatatttgaggagcttcctgggagtgagtcagaaggcgagtgccgtttaaaggctgcaagagaagccatgctggtgaagcggacccttccacctcgggatgtttcaggactaggctgagggcaaaggaaactgccaccacctccctacacctccccaccctccagcacccccaccccaccctggccacacaaccccgctccagtgctcatcccaccgtgaggacgtggaggccggaaggagccgccacacggccctgccctgcagatgtggttgaaggagtctccacgggaatcatgactcccagagcgaggctggggcttggggcgccggggaggcagcttggatttaggagccccagggccaagtctttgccgtgaactgttctggcccctgtgaccaggccctgccccgtgtctccccagggccccggtcccctgtgtaaaaagcagtggtgaacggttggacctcctgacgcccaagttcttgagtttccaaatctgtgatttaaagctgagcccaaatgtgctgggtaccagctggacactcagctccatgtggagccaggaagtggggtctgtggagaggagcgcagaggggcaagacctggggtgggcgtggaaaagcacgggggcgtgacccggagaaggagtgaaggactgttggtgtgcaagggcgtctccatgacgacccgaagaagctaggcatgtcgtggagcgctgagtcctttgcgtcgctaaggggaccaagtggagctgggccaggagaggagatggtcgtggctgggagatggcacccacacatctgaccgggcatgaccagggccttggcaggaaaagcagtcaccaagggcgggtgggcagcccccacccccacagggcagctgctggaggactggcagccagccagccccgttccttttggctccctgaaggggtttacagatgacctgcctatacttgagtctagggtctgtttgcacacttgccggcaggaccctcacccaggctgggtcacactgaagcccaggccagaggaaaaacacagggtttccacaaaggagctgccgcaatgagggtttccttaaggaacagccctggctctcaagggttaaaggataaggcacagcagacagaggtgggctagacaaggacagatggaaatttggtgtctactggtcgccccaggcaggaatgactcagaaggaagcctggccgtcctggttccatgccacagggaaaggcaactgggtcgaaataggccttggtctccagcactatcagtgaccccagggaggtgacaggctggagcaagtgcagggcaggcaggggaggggacgccggccacagcgcactccacggggaagggtctttatgggcccctcctcggagaacccccggtctatctgtcagtctgggacaggccacctcaacttgccaccgaggacaccaaaactctccacagacccctctgcccctctgggaaaccccactgtgctccaggacactcaaaaggaaaggatccctggacaagaggtcctgccaggaacatcagccaaattttggccaacgaccagcaaggtgcacagggaagagcaggggctgaaactcagaggtccagcatcagcgacgcccttggcagcccagggaacacaggcaacgccttttggctctggagtcttaggctcttcatcggcaaactgagcccagggggaaggggctactacgtagggttgtcatgaggatgaaacgagacagcatctggtgtaaagtagaaaaggcataaagggccgggcgcggtggctcacgctgtaatcccagcacttttggaggcccaggcgggtggatcacctgaggtcaggagttcaagaccagcttggccaaccctgtctccactaaaaataaaaaattttgccgggcgtggtggcgagcgcctgtaattccagctactcgggaggctgaggtaggagaatggcttgaacctgggaggcagaggttgcagggagccgaaatggcagcactctagcttgggtgacagagcaagactctgtctaaaaaaaaaagaaaagccataaagacgtgtttgagaaagaggcctgggaagacgggggaaggagggtgattgaacccggaatggcacttgtgtcggcccagggtcatatcccttcatctaaggatcctcgtgcctctaaaaagccaccccgtgcttcctgtgggtttgcaagggctggcttggtgtattcagaatgtggcttgctgcatgaacggaccccgagggccatggccctagagcaggggctcgctccagcggacagctctgcctcaccgctccctgcctgtgagtcccgccacgcccttggtttctgggctcagccgtggaggcagaggctggcctggcagaggctggcctggcagtgcttgacacgcaagtgatttgtgtcttcattgctaaggacaagaggcaatgagaggacaagaagtggttggccttttgtacgctcaacgggtggttttgctactctgtgtcttttctctgatttcacggtgctgttaagtgcttaaaatatgcacatcgtgtagctcacagagccacttctctgaaggccaggacagagaccttataggctctctctccccctagtttcagccttttaccttaaatatacgtctttcttactgctaggctgagttcccgccccagcatgttctgagaaattgagtcaaaataactgagtctgttggcacctcatcgacgatttcttcatagacggtttttttattgttgctgttgttgttggttttttgggtttgtttgtttgttttttgagacagagtttctctctgtcccccaggctgcagtgcagtggcgtggtctcagctcagtgcagcctctgcctcccgggttcaagagattctcctgcctcagcctcccgagtagctgggattatagacgcccaacaccacagcggctaatgtttgtatttttagtagagatggggtttcaccatgttggccaggctggtctcgaactcctgacctcaggtgatccgctcgcctcggctcccaaagtgctgggattataggcgtgagctactgtgcctggccctacttcatagaggtttaaatgccttttcacccttttcctggagactctgaagaagtctcaggaactgggcatttgtgttgcacgtgaggccttgcaatggcggccctgcttggaggaagggcactggcctgggttgcccgcagctccactccccgtgtatgtgtttagggaccacagaggacagacatcgactctctgtagagatgccgccccgcccaggttgcagtttaggttccaaaagtccagtggccagtggattttgggggaatttggaataagaaacagcctagactttggagttgttcattcacttgcagaatttctactcatgccagctgctctggacaggaagatgaatgcgtcacagttcctgcttttcaaagctctctaagttaagtgacttgtttaagatcatagaacccataagtgaggcagctgggactagaacccaggtctcctgactcactgcagcacacagcctttcggcaatctccaaaccagcccagcccaccgacggagggaagaacagaagcattcacacaccctgctgagacagccattcattcattcatttgttaattaaaccaccatttaggaaacgcctgccttaagttcctgacattgttctaggacacagcactggatgcacacagtgaagagtgaaacagacgtggcccagtctcttggcactaaaatcttggtgcagacagacatcaaataattacggaaatgttctcaactgcacatgtggtaaatgcagtgtggaaaagtacagggtgtgctgagagctgcatttcgaatggccagagagtaggggaggtgcatctgactgacaagtcaggaagggccctgtgaggaaccgttctgcggggagctgaggcctgaggctgaggacagccaggtggagaaggtgccaggcctgagcaggcagaggcggagctcatggagaggcaggaaagagcttggccccttggaggacttgaaagagaaggcagg

SEQ ID NO: 114 - DNA, 251 bp, Homo sapiens
aagaaaaaaataattagaatgtcttgttttttaaatgactcttcattttgttgttaatatctggttttaattagaaattgcggttacaatatggaagacttgaatttaaaggtaaagctcttggataacaggtttaagggaaggaaacttccgtttcttagagcactaggtagaattctaatgataaaagaggctctcacgcacaaagactggattctctcttagttttgaaaatgttcagacacagaaat

SEQ ID NO: 115 - DNA, 251 bp, Homo sapiens
tctcagctccctatccataaaacagagggacgaataaactctcctcctaccactaagaggtgtagccagagttaataccctcatcgtcctttgagctcagcagatgaaaggcactgagaaaagtacaaagaatttttatgtgctattgactttattttattttatgtgggggagggagccggccccagctggaaagctgctttctctgaatcaaagggcaggaacccagcaagtttctcaggattggggcc

SEQ ID NO: 116 - DNA, 23 bp, Artificial Sequence
  Description of Artificial Sequence Synthetic oligonucleotide
ctttgagctcagcagatgaaagg

SEQ ID NO: 117 - DNA, 23 bp, Artificial Sequence
  Description of Artificial Sequence Synthetic oligonucleotide
ctgagctcaaaggacgatgaggg

SEQ ID NO: 118 - DNA, 23 bp, Artificial Sequence
  Description of Artificial Sequence Synthetic oligonucleotide
gacgatgagggtattaactctgg

SEQ ID NO: 119 - DNA, 23 bp, Artificial Sequence
  Description of Artificial Sequence Synthetic oligonucleotide
actctcctcctaccactaagagg

SEQ ID NO: 120 - DNA, 23 bp, Artificial Sequence
  Description of Artificial Sequence Synthetic oligonucleotide
tattttattttatgtgggggagg

SEQ ID NO: 121 - DNA, 151 bp, Homo sapiens
ccaaatttcccaaagctacacacacgagattatacacatcaggcactggaactatctgtaatactggaacctaaataaaacaaagcagccaaaaattatgcttggttgtttcatttttgtttaatccagattttccaaaatttatcacatt

SEQ ID NO: 122 - DNA, 23 bp, Artificial Sequence
  Description of Artificial Sequence Synthetic oligonucleotide
actggaactatctgtaatactgg

SEQ ID NO: 123 - DNA, 23 bp, Artificial Sequence
  Description of Artificial Sequence Synthetic oligonucleotide
aagcagccaaaaattatgcttgg

SEQ ID NO: 124 - DNA, 23 bp, Artificial Sequence
  Description of Artificial Sequence Synthetic oligonucleotide
agattatacacatcaggcactgg

SEQ ID NO: 125 - DNA, 23 bp, Artificial Sequence
  Description of Artificial Sequence Synthetic oligonucleotide
gtgtgtagctttgggaaatttgg

SEQ ID NO: 126 - DNA, 23 bp, Artificial Sequence
  Description of Artificial Sequence Synthetic oligonucleotide
aaacaaccaagcataatttttgg

SEQ ID NO: 127 - DNA, 23 bp, Artificial Sequence
  Description of Artificial Sequence Synthetic oligonucleotide
cacacgagattatacacatcagg

SEQ ID NO: 128 - DNA, 23 bp, Artificial Sequence
  Description of Artificial Sequence Synthetic oligonucleotide
ccgcgcgacaaacctgagccatc

SEQ ID NO: 129 - DNA, 54 bp, Homo sapiens
gccacccctcttcctgtcaccgacacttcctcagcatccacaggtcacgccacc

SEQ ID NO: 130 - DNA, 29 bp, Artificial Sequence
  Description of Artificial Sequence Synthetic oligonucleotide
aggtgtagccagagttaataccctcatcg

SEQ ID NO: 131 - DNA, 15 bp, Artificial Sequence
  Description of Artificial Sequence Synthetic oligonucleotide
gatgaaaggcactga

SEQ ID NO: 132 - DNA, 15 bp, Artificial Sequence
  Description of Artificial Sequence Synthetic oligonucleotide
gatgaaagtcactga

SEQ ID NO: 133 - DNA, 39 bp, Artificial Sequence
  Description of Artificial Sequence Synthetic oligonucleotide
cacatcaggcactggaactatctgtaatactggaaccta

SEQ ID NO: 134 - DNA, 39 bp, Artificial Sequence
  Description of Artificial Sequence Synthetic oligonucleotide
cacatcaggcactggaactatctgtaataattgaaccta

SEQ ID NO: 135 - DNA, 23 bp, Artificial Sequence
  Description of Artificial Sequence Synthetic oligonucleotide
aaccagccaaaaattatgcttgg